SEQUENCE LISTING (667 sequences)

SEQ ID NO: 1 - DNA, 1542 bp, Artificial Sequence
  Synthetic DNA
atggccgattctcaacctctgtctggagcacctgaaggagcagaatatttacgggcagtgttacgtgcgccggtgtatgaagccgcccaggtgaccccgttacagaaaatggaaaaactcagttcccgtctcgataatgtgattctggtcaagcgcgaggaccgacagcccgtgcactcgttcaagctccgtggtgcgtatgcgatgatggcagggttgacggaagaacagaaagcccacggtgtgattacggcatcagctggcaaccatgctcaaggtgtggcgttctcttctgctcgactgggagtgaaagcgttaatcgtgatgcctactgctacagcggatattaaagtggatgccgtccgagggtttggtggtgaagttctgctgcatggcgcgaactttgatgaagccaaggccaaggcgatcgagctctctcaacaacaggggttcacgtgggtgccaccattcgatcatccgatggtaatcgccggtcaggggacgttagcactggagttgcttcaacaggacgcacatctcgaccgggtcttcgttcctgttgggggtggtggtctggcggcgggtgtagcagtactcatcaagcagctcatgccacaaattaaagtgatagccgttgaagccgaagattccgcatgtctgaaggccgcacttgatgccggacaccctgtcgatctgccgcgtgtggggctgtttgcagaaggggttgcggtgaaacggattggggatgagaccttccgcctatgccaggagtatttggacgacatcatcaccgtggactccgatgccatttgtgccgccatgaaggacctattcgaagatgtccgtgcagtcgccgaaccgtctggagctttagcattagccgggatgaagaagtacattgctctgcacaacatccgaggcgaacgactggcccacatcttaagcggtgcgaatgtcaacttccacggcttacggtatgtgtctgagcgttgcgagctgggcgaacaaagagaagcattactggcagtgaccattccggaagaaaaaggttcgttcctcaagttctgccagctgttaggaggtcggagcgtcacggaatttaactatcggtttgcagacgccaagaatgcctgtatttttgtgggtgtgaggttgagcaggggattggaggagcgcaaggagattcttcagatgctgaacgatggcggttatagcgtggtggacctgagcgacgacgaaatggctaaactacacgtacgctacatggtgggtggacgaccttcacatcccctccaggagcgactgtattcctttgaattcccagagtctcccggcgccttattacgtttcttaaacaccctgggcacctattggaatatcagcctgttccactaccgatctcatgggacggattacgggcgtgttctgctggcgtttgagcttggcgatcatgaaccggactttgaaacgcgcctgaacgaactgggctatgattgccatgatgagaccaacaaccccgcctttcgtttcttcctcgcaggc

SEQ ID NO: 2 - DNA, 50 bp, Artificial Sequence
  Synthetic DNA
ctatactgcagatggccgattctcaaccactgtctggagctcctgaaggg

SEQ ID NO: 3 - DNA, 50 bp, Artificial Sequence
  Synthetic DNA
gtctggagctcctgaaggggcagaatatttacgggcagtgttacgtgcgc

SEQ ID NO: 4 - DNA, 47 bp, Artificial Sequence
  Synthetic DNA
gggcagtgttacgtgcgccggtgtatgaagccgcccaggtgaccccg

SEQ ID NO: 5 - DNA, 49 bp, Artificial Sequence
  Synthetic DNA
gccgcccaggtgaccccgttacagaaaatggaaaaactctcctcccggc

SEQ ID NO: 6 - DNA, 49 bp, Artificial Sequence
  Synthetic DNA
gaaaaactctcctcccggctcgataatgtgattctggtcaagcgcgagg

SEQ ID NO: 7 - DNA, 50 bp, Artificial Sequence
  Synthetic DNA
gattctggtcaagcgcgaggaccgtcagcccgtgcactcgttcaagctcc

SEQ ID NO: 8 - DNA, 50 bp, Artificial Sequence
  Synthetic DNA
gtgcactcgttcaagctccgtggtgcctatgcgatgatggcgggcctgac

SEQ ID NO: 9 - DNA, 50 bp, Artificial Sequence
  Synthetic DNA
gatgatggcgggcctgacggaagaacagaaagcccacggtgtgattacgg

SEQ ID NO: 10 - DNA, 50 bp, Artificial Sequence
  Synthetic DNA
cccacggtgtgattacggcatcagcaggcaaccatgctcaaggtgtggcg

SEQ ID NO: 11 - DNA, 49 bp, Artificial Sequence
  Synthetic DNA
catgctcaaggtgtggcgttctcttctgctcgactgggagtgaaagcgc

SEQ ID NO: 12 - DNA, 50 bp, Artificial Sequence
  Synthetic DNA
gactgggagtgaaagcgctgattgtgatgcctacagctactcgagaatac

SEQ ID NO: 13 - DNA, 49 bp, Artificial Sequence
  Synthetic DNA
ctatactgcagagtgaaagcgctgattgtgatgcctacagctacagccg

SEQ ID NO: 14 - DNA, 50 bp, Artificial Sequence
  Synthetic DNA
gatgcctacagctacagccgatattaaagtggatgcggtgcgtggcttcg

SEQ ID NO: 15 - DNA, 48 bp, Artificial Sequence
  Synthetic DNA
gatgcggtgcgtggcttcggtggtgaagttctgctgcatggcgcgaac

SEQ ID NO: 16 - DNA, 49 bp, Artificial Sequence
  Synthetic DNA
ctgctgcatggcgcgaactttgatgaagccaaggccaaggcgatcgagc

SEQ ID NO: 17 - DNA, 50 bp, Artificial Sequence
  Synthetic DNA
caaggccaaggcgatcgagctctctcaacaacaggggttcacgtgggtgc

SEQ ID NO: 18 - DNA, 50 bp, Artificial Sequence
  Synthetic DNA
caggggttcacgtgggtgccaccgtttgatcatccgatggtcatcgccgg

SEQ ID NO: 19 - DNA, 49 bp, Artificial Sequence
  Synthetic DNA
catccgatggtcatcgccggtcaaggcacgttagcgctggagttgcttc

SEQ ID NO: 20 - DNA, 50 bp, Artificial Sequence
  Synthetic DNA
gttagcgctggagttgcttcaacaggacgcacacctcgaccgggtcttcg

SEQ ID NO: 21 - DNA, 50 bp, Artificial Sequence
  Synthetic DNA
cacctcgaccgggtcttcgttcctgttgggggtggtggtctggcggcggg

SEQ ID NO: 22 - DNA, 49 bp, Artificial Sequence
  Synthetic DNA
gtggtggtctggcggcgggggtagcagtactcatcaagcagctcatgcc

SEQ ID NO: 23 - DNA, 49 bp, Artificial Sequence
  Synthetic DNA
catcaagcagctcatgccacaaattaaagtgatagccgttgaagcctcg

SEQ ID NO: 24 - DNA, 26 bp, Artificial Sequence
  Synthetic DNA
gatagccgttgaagcctcgagaatac

SEQ ID NO: 25 - DNA, 48 bp, Artificial Sequence
  Synthetic DNA
ctatactgcagctcatgccacaaattaaagtgatagccgttgaagccg

SEQ ID NO: 26 - DNA, 49 bp, Artificial Sequence
  Synthetic DNA
gtgatagccgttgaagccgaagattccgcatgcctgaaggccgcacttg

SEQ ID NO: 27 - DNA, 50 bp, Artificial Sequence
  Synthetic DNA
gcctgaaggccgcacttgacgccggacatccagtcgacctgccgcgcgtg

SEQ ID NO: 28 - DNA, 50 bp, Artificial Sequence
  Synthetic DNA
gtcgacctgccgcgcgtggggctgtttgcagaaggggttgcggtgaaacg

SEQ ID NO: 29 - DNA, 49 bp, Artificial Sequence
  Synthetic DNA
gaaggggttgcggtgaaacggattggggatgagaccttccgcctatgcc

SEQ ID NO: 30 - DNA, 49 bp, Artificial Sequence
  Synthetic DNA
gagaccttccgcctatgccaggagtatttggacgacatcatcaccgtgg

SEQ ID NO: 31 - DNA, 49 bp, Artificial Sequence
  Synthetic DNA
gacgacatcatcaccgtggactccgatgccatttgtgccgccatgaagg

SEQ ID NO: 32 - DNA, 50 bp, Artificial Sequence
  Synthetic DNA
catttgtgccgccatgaaggacctattcgaggatgtccgtgcagtcgccg

SEQ ID NO: 33 - DNA, 48 bp, Artificial Sequence
  Synthetic DNA
gatgtccgtgcagtcgccgaaccgtctggagctctcgcactggccggg

SEQ ID NO: 34 - DNA, 50 bp, Artificial Sequence
  Synthetic DNA
gctctcgcactggccgggatgaagaagtacattgctctgcacaacatccg

SEQ ID NO: 35 - DNA, 49 bp, Artificial Sequence
  Synthetic DNA
cattgctctgcacaacatccgaggcgaacgactggcccacatcctgagc

SEQ ID NO: 36 - DNA, 28 bp, Artificial Sequence
  Synthetic DNA
ctggcccacatcctgagctcgagaatac

SEQ ID NO: 37 - DNA, 50 bp, Artificial Sequence
  Synthetic DNA
ctatactgcagcacaacatccgaggcgaacgactggcccacatcctgagc

SEQ ID NO: 38 - DNA, 48 bp, Artificial Sequence
  Synthetic DNA
ctggcccacatcctgagcggtgcgaatgtcaacttccacggcttacgg

SEQ ID NO: 39 - DNA, 50 bp, Artificial Sequence
  Synthetic DNA
caacttccacggcttacggtatgtgtctgagcgttgcgagctgggcgaac

SEQ ID NO: 40 - DNA, 50 bp, Artificial Sequence
  Synthetic DNA
gttgcgagctgggcgaacaacgcgaagcattactggcagtgaccattccg

SEQ ID NO: 41 - DNA, 49 bp, Artificial Sequence
  Synthetic DNA
ctggcagtgaccattccggaagaaaaaggttcgttcctcaagttctgcc

SEQ ID NO: 42 - DNA, 45 bp, Artificial Sequence
  Synthetic DNA
cgttcctcaagttctgccagctgttaggaggtcggagcgtcacgg

SEQ ID NO: 43 - DNA, 47 bp, Artificial Sequence
  Synthetic DNA
gaggtcggagcgtcacggaatttaactatcggtttgcagacgccaag

SEQ ID NO: 44 - DNA, 50 bp, Artificial Sequence
  Synthetic DNA
cggtttgcagacgccaagaatgcctgtatttttgtgggtgtgaggttgag

SEQ ID NO: 45 - DNA, 50 bp, Artificial Sequence
  Synthetic DNA
gtatttttgtgggtgtgaggttgagcaggggattggaggagcgcaaggag

SEQ ID NO: 46 - DNA, 46 bp, Artificial Sequence
  Synthetic DNA
gattggaggagcgcaaggagattcttcagatgctgaacgatggcgg

SEQ ID NO: 47 - DNA, 47 bp, Artificial Sequence
  Synthetic DNA
gatgctgaacgatggcggttatagcgtggtggacctgagcgacgacg

SEQ ID NO: 48 - DNA, 36 bp, Artificial Sequence
  Synthetic DNA
gtggacctgagcgacgacgaaatggctcgagaatac

SEQ ID NO: 49 - DNA, 47 bp, Artificial Sequence
  Synthetic DNA
ctatactgcagttatagcgtggtggacctgagcgacgacgaaatggc

SEQ ID NO: 50 - DNA, 50 bp, Artificial Sequence
  Synthetic DNA
gagcgacgacgaaatggctaaactacacgtacgctacatggtgggtggac

SEQ ID NO: 51 - DNA, 47 bp, Artificial Sequence
  Synthetic DNA
gctacatggtgggtggacgaccttcacatcccctccaggagcgactg

SEQ ID NO: 52 - DNA, 50 bp, Artificial Sequence
  Synthetic DNA
cccctccaggagcgactgtattcctttgaattcccagagtctcccggcgc

SEQ ID NO: 53 - DNA, 49 bp, Artificial Sequence
  Synthetic DNA
cccagagtctcccggcgccttattacgtttcttaaacaccctgggcacc

SEQ ID NO: 54 - DNA, 48 bp, Artificial Sequence
  Synthetic DNA
cttaaacaccctgggcacctattggaatatcagcctgttccactaccg

SEQ ID NO: 55 - DNA, 50 bp, Artificial Sequence
  Synthetic DNA
cagcctgttccactaccgatctcacggcacggattacgggcgtgttctgg

SEQ ID NO: 56 - DNA, 49 bp, Artificial Sequence
  Synthetic DNA
gattacgggcgtgttctggcggcgtttgaactgggcgatcatgaaccgg

SEQ ID NO: 57 - DNA, 48 bp, Artificial Sequence
  Synthetic DNA
ctgggcgatcatgaaccggactttgaaacgcgcctgaacgaactgggc

SEQ ID NO: 58 - DNA, 50 bp, Artificial Sequence
  Synthetic DNA
cgcctgaacgaactgggctatgattgccatgatgagaccaacaaccccgc

SEQ ID NO: 59 - DNA, 49 bp, Artificial Sequence
  Synthetic DNA
gatgagaccaacaaccccgcctttcgtttcttcctcgccggctaactcg

SEQ ID NO: 60 - DNA, 27 bp, Artificial Sequence
  Synthetic DNA
cttcctcgccggctaactcgagaatac

SEQ ID NO: 61 - DNA, 36 bp, Artificial Sequence
  Synthetic DNA
ggccgattctcaacctctgtctggagcacctgaagg

SEQ ID NO: 62 - DNA, 50 bp, Artificial Sequence
  Synthetic DNA
gcgcacgtaacactgcccgtaaatattctgctccttcaggtgctccagac

SEQ ID NO: 63 - DNA, 47 bp, Artificial Sequence; identical to SEQ ID NO: 4
  Synthetic DNA

SEQ ID NO: 64 - DNA, 49 bp, Artificial Sequence
  Synthetic DNA
gacgggaactgagtttttccattttctgtaacggggtcacctgggcggc

SEQ ID NO: 65 - DNA, 49 bp, Artificial Sequence
  Synthetic DNA
gaaaaactcagttcccgtctcgataatgtgattctggtcaagcgcgagg

SEQ ID NO: 66 - DNA, 50 bp, Artificial Sequence
  Synthetic DNA
ggagcttgaacgagtgcacgggctgtcggtcctcgcgcttgaccagaatc

SEQ ID NO: 67 - DNA, 50 bp, Artificial Sequence
  Synthetic DNA
gtgcactcgttcaagctccgtggtgcgtatgcgatgatggcagggttgac

SEQ ID NO: 68 - DNA, 50 bp, Artificial Sequence
  Synthetic DNA
ccgtaatcacaccgtgggctttctgttcttccgtcaaccctgccatcatc

SEQ ID NO: 69 - DNA, 50 bp, Artificial Sequence
  Synthetic DNA
cccacggtgtgattacggcatcagctggcaaccatgctcaaggtgtggcg

SEQ ID NO: 70 - DNA, 48 bp, Artificial Sequence
  Synthetic DNA
cgctttcactcccagtcgagcagaagagaacgccacaccttgagcatg

SEQ ID NO: 71 - DNA, 41 bp, Artificial Sequence
  Synthetic DNA
cgactgggagtgaaagcgttaatcgtgatgcctactgctac

SEQ ID NO: 72 - DNA, 39 bp, Artificial Sequence
  Synthetic DNA
gagtgaaagcgttaatcgtgatgcctactgctacagcgg

SEQ ID NO: 73 - DNA, 50 bp, Artificial Sequence
  Synthetic DNA
caaaccctcggacggcatccactttaatatccgctgtagcagtaggcatc

SEQ ID NO: 74 - DNA, 48 bp, Artificial Sequence
  Synthetic DNA
gatgccgtccgagggtttggtggtgaagttctgctgcatggcgcgaac

SEQ ID NO: 75 - DNA, 49 bp, Artificial Sequence
  Synthetic DNA
gctcgatcgccttggccttggcttcatcaaagttcgcgccatgcagcag

SEQ ID NO: 76 - DNA, 50 bp, Artificial Sequence; identical to SEQ ID NO: 17
  Synthetic DNA

SEQ ID NO: 77 - DNA, 50 bp, Artificial Sequence
  Synthetic DNA
ccggcgattaccatcggatgatcgaatggtggcacccacgtgaacccctg

SEQ ID NO: 78 - DNA, 49 bp, Artificial Sequence
  Synthetic DNA
catccgatggtaatcgccggtcaggggacgttagcactggagttgcttc

SEQ ID NO: 79 - DNA, 50 bp, Artificial Sequence
  Synthetic DNA
cgaagacccggtcgagatgtgcgtcctgttgaagcaactccagtgctaac

SEQ ID NO: 80 - DNA, 50 bp, Artificial Sequence
  Synthetic DNA
catctcgaccgggtcttcgttcctgttgggggtggtggtctggcggcggg

SEQ ID NO: 81 - DNA, 49 bp, Artificial Sequence
  Synthetic DNA
ggcatgagctgcttgatgagtactgctacacccgccgccagaccaccac

SEQ ID NO: 82 - DNA, 46 bp, Artificial Sequence
  Synthetic DNA
catcaagcagctcatgccacaaattaaagtgatagccgttgaagcc

SEQ ID NO: 83 - DNA, 38 bp, Artificial Sequence
  Synthetic DNA
gctcatgccacaaattaaagtgatagccgttgaagccg

SEQ ID NO: 84 - DNA, 49 bp, Artificial Sequence
  Synthetic DNA
caagtgcggccttcagacatgcggaatcttcggcttcaacggctatcac

SEQ ID NO: 85 - DNA, 50 bp, Artificial Sequence
  Synthetic DNA
gtctgaaggccgcacttgatgccggacaccctgtcgatctgccgcgtgtg

SEQ ID NO: 86 - DNA, 50 bp, Artificial Sequence
  Synthetic DNA
cgtttcaccgcaaccccttctgcaaacagccccacacgcggcagatcgac

SEQ ID NO: 87 - DNA, 49 bp, Artificial Sequence; identical to SEQ ID NO: 29
  Synthetic DNA

SEQ ID NO: 88 - DNA, 49 bp, Artificial Sequence
  Synthetic DNA
ccacggtgatgatgtcgtccaaatactcctggcataggcggaaggtctc

SEQ ID NO: 89 - DNA, 49 bp, Artificial Sequence; identical to SEQ ID NO: 31
  Synthetic DNA

SEQ ID NO: 90 - DNA, 50 bp, Artificial Sequence
  Synthetic DNA
cggcgactgcacggacatcttcgaataggtccttcatggcggcacaaatg

SEQ ID NO: 91 - DNA, 48 bp, Artificial Sequence
  Synthetic DNA
gatgtccgtgcagtcgccgaaccgtctggagctttagcattagccggg

SEQ ID NO: 92 - DNA, 50 bp, Artificial Sequence
  Synthetic DNA
cggatgttgtgcagagcaatgtacttcttcatcccggctaatgctaaagc

SEQ ID NO: 93 - DNA, 49 bp, Artificial Sequence
  Synthetic DNA
cattgctctgcacaacatccgaggcgaacgactggcccacatcttaagc

SEQ ID NO: 94 - DNA, 40 bp, Artificial Sequence
  Synthetic DNA
gcacaacatccgaggcgaacgactggcccacatcttaagc

SEQ ID NO: 95 - DNA, 48 bp, Artificial Sequence
  Synthetic DNA
ccgtaagccgtggaagttgacattcgcaccgcttaagatgtgggccag

SEQ ID NO: 96 - DNA, 50 bp, Artificial Sequence; identical to SEQ ID NO: 39
  Synthetic DNA

SEQ ID NO: 97 - DNA, 50 bp, Artificial Sequence
  Synthetic DNA
cggaatggtcactgccagtaatgcttctctttgttcgcccagctcgcaac

SEQ ID NO: 98 - DNA, 49 bp, Artificial Sequence; identical to SEQ ID NO: 41
  Synthetic DNA

SEQ ID NO: 99 - DNA, 45 bp, Artificial Sequence
  Synthetic DNA
ccgtgacgctccgacctcctaacagctggcagaacttgaggaacg

SEQ ID NO: 100 - DNA, 47 bp, Artificial Sequence; identical to SEQ ID NO: 43
  Synthetic DNA

SEQ ID NO: 101 - DNA, 50 bp, Artificial Sequence
  Synthetic DNA
ctcaacctcacacccacaaaaatacaggcattcttggcgtctgcaaaccg

SEQ ID NO: 102 - DNA, 50 bp, Artificial Sequence; identical to SEQ ID NO: 45
  Synthetic DNA

SEQ ID NO: 103 - DNA, 46 bp, Artificial Sequence
  Synthetic DNA
ccgccatcgttcagcatctgaagaatctccttgcgctcctccaatc

SEQ ID NO: 104 - DNA, 47 bp, Artificial Sequence; identical to SEQ ID NO: 47
  Synthetic DNA

SEQ ID NO: 105 - DNA, 26 bp, Artificial Sequence
  Synthetic DNA
gccatttcgtcgtcgctcaggtccac

SEQ ID NO: 106 - DNA, 37 bp, Artificial Sequence
  Synthetic DNA
gttatagcgtggtggacctgagcgacgacgaaatggc

SEQ ID NO: 107 - DNA, 50 bp, Artificial Sequence
  Synthetic DNA
gtccacccaccatgtagcgtacgtgtagtttagccatttcgtcgtcgctc

SEQ ID NO: 108 - DNA, 47 bp, Artificial Sequence; identical to SEQ ID NO: 51
  Synthetic DNA

SEQ ID NO: 109 - DNA, 50 bp, Artificial Sequence
  Synthetic DNA
gcgccgggagactctgggaattcaaaggaatacagtcgctcctggagggg

SEQ ID NO: 110 - DNA, 49 bp, Artificial Sequence; identical to SEQ ID NO: 53
  Synthetic DNA

SEQ ID NO: 111 - DNA, 48 bp, Artificial Sequence
  Synthetic DNA
cggtagtggaacaggctgatattccaataggtgcccagggtgtttaag

SEQ ID NO: 112 - DNA, 50 bp, Artificial Sequence
  Synthetic DNA
cagcctgttccactaccgatctcatgggacggattacgggcgtgttctgg

SEQ ID NO: 113 - DNA, 49 bp, Artificial Sequence
  Synthetic DNA
ccggttcatgatcgccaagctcaaacgctgccagaacacgcccgtaatc

SEQ ID NO: 114 - DNA, 48 bp, Artificial Sequence
  Synthetic DNA
cttggcgatcatgaaccggactttgaaacgcgcctgaacgaactgggc

SEQ ID NO: 115 - DNA, 50 bp, Artificial Sequence
  Synthetic DNA
gcggggttgttggtctcatcatggcaatcatagcccagttcgttcaggcg

SEQ ID NO: 116 - DNA, 45 bp, Artificial Sequence
  Synthetic DNA
gatgagaccaacaaccccgcctttcgtttcttcctcgcaggctaa

SEQ ID NO: 117 - DNA, 17 bp, Artificial Sequence
  Synthetic DNA
gtagcagtaggcatcac

SEQ ID NO: 118 - DNA, 18 bp, Artificial Sequence
  Synthetic DNA
ggcttcaacggctatcac

SEQ ID NO: 119 - DNA, 18 bp, Artificial Sequence
  Synthetic DNA
gcttaagatgtgggccag

SEQ ID NO: 120 - DNA, 20 bp, Artificial Sequence
  Synthetic DNA
ttagcctgcgaggaagaaac

SEQ ID NO: 121 - DNA, 45 bp, Artificial Sequence
  Synthetic DNA
ctatatctagcatatggccgattctcaacctctgtctggagcacc

SEQ ID NO: 122 - DNA, 45 bp, Artificial Sequence
  Synthetic DNA
gtattggatccttagcctgcgaggaagaaacgaaaggcggggttg

SEQ ID NO: 123 - DNA, 1512 bp, Artificial Sequence
  Synthetic DNA
atggatgtgcgttgcatcaattggtttgaatcgcatggtgaaaacaggtttttatatctgaaaagccgctgtcgtaatggggaaactgtgttcattcgcttccctcactacttttactatgtggtgaccgatgagatctaccagagcttagcccccccacctttcaacgctcgtcctatgggtaaaatgcggaccattgacatcgatgagaccatctcgtacaacctggacatcaaggatcgtaaatgctctgtggcggacatgtggttaattgaagagccgaaaaagcgcaacattcagaatgccaccatggatgagtttctgaatatttcttggttctacatcagcaacggcatttctccggatggatgctacagcttggacgatcagtatctcacgaaaatcaacaacgggtgctatcattgtggcgaccctcgtaactgttttgcgaaagagatcccccgttttgacattccgagaagctatctgttcctggacattgaatgccatttcgataagaagttcccgagcgtttttattaatccgatcagccatacctcctattgttatattgatctgagcggcaaacgtctgctgtttaccctgatcaacgaggagatgctgaccgaacaagaaatccaggaggccgtggatcgtggctgtctgcgcattcagtccttgatggagatggattatgaacgtgaactggtgctgtgctctgaaattgtgctgctccaaatcgccaaacagttattagagctgacctttgattacatcgtgacgttcaacggccacaacttcgatctgcggtatattaccaatcgtctcgagctgttgaccggcgaaaaaatcatctttcgtagccccgacaagaaagaagcggttcacctgtgcatctatgagcgtaatcagtcgagccacaaaggggttggagggatggcgaatacgaccttccacgtcaataataataatggcaccatttttttcgacctgtattctttcatccagaaatcggagaagcttgattcttacaaactggacagcatcagcaaaaacgccttttcgtgcatgggcaaagtgctgaatcgtggtgtgcgtgagatgacctttatcggtgatgataccactgatgcgaaagggaaagcggctgtgtttgcgaaggtcctcaccacaggcaattacgtgacggtcgatgatatcatttgtaaagtgattcacaaggacatctgggaaaatggctttaaggtggtgttgagctgtccgactctgaccaacgacacgtacaaactctcctttggtaaagatgatgtcgacctggcgcagatgtataaagactataacctgaacatcgcccttgatatggcccgctattgcatccacgacgcctgtctgtgccaatacctgtgggagtactatggtgtagagacgaaaacggatgcgggtgcctctacctatgtgttgcctcagtccatggtgtttgagtataaagcgagcacggtgattaaggggccc

SEQ ID NO: 124 - DNA, 1509 bp, Artificial Sequence
  Synthetic DNA
gggcccctgctgaaattgctgctggaaaccaagaccatcttagtgcgctctgaaaccaaacaaaagttcccctatgaaggcggtaaagtttttgccccgaagcagaagatgtttagtaacaacgtcctgatctttgactacaactctctgtatcccaacgtgtgcatctttggcaacttaagtccggaaaccctggttggcgtggtggtgtcttcgaaccgcttggaagaagagattaacaaccagctgctcctgcaaaagtacccgccgccacgttacattacggtgcactgcgaaccacgtttacccaacctgatcagcgagattgccatttttgatcggagcattgaaggcaccattccgcgtttactgagaacctttctggccgagcgtgcgcgttataagaaaatgctgaaacaggcgaccagttctacggaaaaagccatctacgacagtatgcagtacacctacaagatcatcgcgaatagtgtgtatggcttgatgggttttcgcaactctgccttgtatagctatgccagcgctaagagttgtaccagtattggccgtcgtatgatcctgtatctggaatctgtactcaatggagcggaactgagtaatggcatgcttcgttttgcaaacccgttaagtaatccgttctacatggatgatcgcgacattaacccgattgtgaagacgtccctgccgattgactaccgttttcgcttcaggagtgtctatggtgataccgactccgtgtttaccgaaattgacagccaggatgttgacaaaagtattgagatagcgaaggagctggaacgtctgatcaactctcgtgtgctgttcaacaactttaagatcgagtttgaggccgtgtataaaaacctgatcatgcagagcaagaaaaaatataccacgatgaagtatagcgcgagttctaactccaaaagtgtgccggagcgtattaacaaggggactagcgaaacccgtcgtgatgtcagcaagttccacaaaaacatgattaaaatttacaagacccgtttgagcgaaatgttaagtgaaggccggatgaacagcaaccaggtgtgtatcgacattctgcgttcccttgaaacggatcttcgtagcgagttcgacagccgatctagcccgttggaactgttcatgttaagccgcatgcaccacttgaactataaaagcgccgataacccgaacatgtacctggtgaccgagtacaacaaaaacaacccggaaactattgaacttggcgaacgctactactttgcctatatctgtccggcgaatgttccgtggaccaaaaaactcgtgaacatcaagacgtacgaaaccattattgaccgttccttcaagctgggctcagatcagcgcattttttacgaggtgtattttaaacgtctgacctccgaaatcgtgaacctgttagataacaaggtgctgtgcatttctttttttgaacgcatgtttggcagcagaccgaccttctatgaggcg

SEQ ID NO: 125 - DNA, 30 bp, Artificial Sequence
  Synthetic DNA
tcctcgagcataatggatgtgcgttgcatc

SEQ ID NO: 126 - DNA, 48 bp, Artificial Sequence
  Synthetic DNA
gacgacgacgacaagcatatgctcgaggatatggatgtgcgttgcatc

SEQ ID NO: 127 - DNA, 48 bp, Artificial Sequence
  Synthetic DNA
atggatgtgcgttgcatcaattggtttgaatcgcatggtgaaaacagg

SEQ ID NO: 128 - DNA, 50 bp, Artificial Sequence
  Synthetic DNA
cgcatggtgaaaacaggtttttatatctgaaaagccgctgtcgtaatggg

SEQ ID NO: 129 - DNA, 47 bp, Artificial Sequence
  Synthetic DNA
gccgctgtcgtaatggggaaactgtgttcattcgcttccctcactac

SEQ ID NO: 130 - DNA, 50 bp, Artificial Sequence
  Synthetic DNA
cattcgcttccctcactacttttactatgtggtgaccgatgagatctacc

SEQ ID NO: 131 - DNA, 50 bp, Artificial Sequence
  Synthetic DNA
ggtgaccgatgagatctaccagagcttagcccccccacctttcaacgctc

SEQ ID NO: 132 - DNA, 50 bp, Artificial Sequence
  Synthetic DNA
cccacctttcaacgctcgtcctatgggtaaaatgcggaccattgacatcg

SEQ ID NO: 133 - DNA, 50 bp, Artificial Sequence
  Synthetic DNA
gcggaccattgacatcgatgagaccatctcgtacaacctggacatcaagg

SEQ ID NO: 134 - DNA, 49 bp, Artificial Sequence
  Synthetic DNA
cgtacaacctggacatcaaggatcgtaaatgctctgtggcggacatgtg

SEQ ID NO: 135 - DNA, 46 bp, Artificial Sequence
  Synthetic DNA
ctctgtggcggacatgtggttaattgaagagccgaaaaagcgcaac

SEQ ID NO: 136 - DNA, 48 bp, Artificial Sequence
  Synthetic DNA
gagccgaaaaagcgcaacattcagaatgccaccatggatgagtttctg

SEQ ID NO: 137 - DNA, 50 bp, Artificial Sequence
  Synthetic DNA
ccaccatggatgagtttctgaatatttcttggttctacatcagcaacggc

SEQ ID NO: 138 - DNA, 50 bp, Artificial Sequence
  Synthetic DNA
ggttctacatcagcaacggcatttctccggatggatgctacagcttggac

SEQ ID NO: 139 - DNA, 50 bp, Artificial Sequence; identical to SEQ ID NO: 138
  Synthetic DNA

SEQ ID NO: 140 - DNA, 50 bp, Artificial Sequence
  Synthetic DNA
ggatgctacagcttggacgatcagtatctcacgaaaatcaacaacgggtg

SEQ ID NO: 141 - DNA, 48 bp, Artificial Sequence
  Synthetic DNA
cacgaaaatcaacaacgggtgctatcattgtggcgaccctcgtaactg

SEQ ID NO: 142 - DNA, 45 bp, Artificial Sequence
  Synthetic DNA
ggcgaccctcgtaactgttttgcgaaagagatcccccgttttgac

SEQ ID NO: 143 - DNA, 49 bp, Artificial Sequence
  Synthetic DNA
gagatcccccgttttgacattccgagaagctatctgttcctggacattg

SEQ ID NO: 144 - DNA, 50 bp, Artificial Sequence
  Synthetic DNA
gctatctgttcctggacattgaatgccatttcgataagaagttcccgagc

SEQ ID NO: 145 - DNA, 49 bp, Artificial Sequence
  Synthetic DNA
cgataagaagttcccgagcgtttttattaatccgatcagccatacctcc

SEQ ID NO: 146 - DNA, 50 bp, Artificial Sequence
  Synthetic DNA
cgatcagccatacctcctattgttatattgatctgagcggcaaacgtctg

SEQ ID NO: 147 - DNA, 50 bp, Artificial Sequence
  Synthetic DNA
ctgagcggcaaacgtctgctgtttaccctgatcaacgaggagatgctgac

SEQ ID NO: 148 - DNA, 50 bp, Artificial Sequence
  Synthetic DNA
gatcaacgaggagatgctgaccgaacaagaaatccaggaggccgtggatc

SEQ ID NO: 149 - DNA, 49 bp, Artificial Sequence
  Synthetic DNA
ccaggaggccgtggatcgtggctgtctgcgcattcagtccttgatggag

SEQ ID NO: 150 - DNA, 50 bp, Artificial Sequence
  Synthetic DNA
gcattcagtccttgatggagatggattatgaacgtgaactggtgctgtgc

SEQ ID NO: 151 - DNA, 50 bp, Artificial Sequence; identical to SEQ ID NO: 150
  Synthetic DNA

SEQ ID NO: 152 - DNA, 48 bp, Artificial Sequence
  Synthetic DNA
gtgaactggtgctgtgctctgaaattgtgctgctccaaatcgccaaac

SEQ ID NO: 153 - DNA, 49 bp, Artificial Sequence
  Synthetic DNA
gctccaaatcgccaaacagttattagagctgacctttgattacatcgtg

SEQ ID NO: 154 - DNA, 50 bp, Artificial Sequence
  Synthetic DNA
gctgacctttgattacatcgtgacgttcaacggccacaacttcgatctgc

SEQ ID NO: 155 - DNA, 50 bp, Artificial Sequence
  Synthetic DNA
gccacaacttcgatctgcggtatattaccaatcgtctcgagctgttgacc

SEQ ID NO: 156 - DNA, 50 bp, Artificial Sequence
  Synthetic DNA
gtctcgagctgttgaccggcgaaaaaatcatctttcgtagccccgacaag

SEQ ID NO: 157 - DNA, 50 bp, Artificial Sequence
  Synthetic DNA
ctttcgtagccccgacaagaaagaagcggttcacctgtgcatctatgagc

SEQ ID NO: 158 - DNA, 46 bp, Artificial Sequence
  Synthetic DNA
cacctgtgcatctatgagcgtaatcagtcgagccacaaaggggttg

SEQ ID NO: 159 - DNA, 46 bp, Artificial Sequence
  Synthetic DNA
gagccacaaaggggttggagggatggcgaatacgaccttccacgtc

SEQ ID NO: 160 - DNA, 50 bp, Artificial Sequence
  Synthetic DNA
gaatacgaccttccacgtcaataataataatggcaccatttttttcgacc

SEQ ID NO: 161 - DNA, 50 bp, Artificial Sequence; identical to SEQ ID NO: 160
  Synthetic DNA

SEQ ID NO: 162 - DNA, 49 bp, Artificial Sequence
  Synthetic DNA
ggcaccatttttttcgacctgtattctttcatccagaaatcggagaagc

SEQ ID NO: 163 - DNA, 49 bp, Artificial Sequence
  Synthetic DNA
catccagaaatcggagaagcttgattcttacaaactggacagcatcagc

SEQ ID NO: 164 - DNA, 50 bp, Artificial Sequence
  Synthetic DNA
caaactggacagcatcagcaaaaacgccttttcgtgcatgggcaaagtgc

SEQ ID NO: 165 - DNA, 49 bp, Artificial Sequence
  Synthetic DNA
gtgcatgggcaaagtgctgaatcgtggtgtgcgtgagatgacctttatc

SEQ ID NO: 166 - DNA, 48 bp, Artificial Sequence
  Synthetic DNA
gtgcgtgagatgacctttatcggtgatgataccactgatgcgaaaggg

SEQ ID NO: 167 - DNA, 47 bp, Artificial Sequence
  Synthetic DNA
ccactgatgcgaaagggaaagcggctgtgtttgcgaaggtcctcacc

SEQ ID NO: 168 - DNA, 49 bp, Artificial Sequence
  Synthetic DNA
gtttgcgaaggtcctcaccacaggcaattacgtgacggtcgatgatatc

SEQ ID NO: 169 - DNA, 50 bp, Artificial Sequence
  Synthetic DNA
cgtgacggtcgatgatatcatttgtaaagtgattcacaaggacatctggg

SEQ ID NO: 170 - DNA, 50 bp, Artificial Sequence
  Synthetic DNA
gattcacaaggacatctgggaaaatggctttaaggtggtgttgagctgtc

SEQ ID NO: 171 - DNA, 50 bp, Artificial Sequence; identical to SEQ ID NO: 170
  Synthetic DNA

SEQ ID NO: 172 - DNA, 49 bp, Artificial Sequence
  Synthetic DNA
ggtggtgttgagctgtccgactctgaccaacgacacgtacaaactctcc

SEQ ID NO: 173 - DNA, 49 bp, Artificial Sequence
  Synthetic DNA
cgacacgtacaaactctcctttggtaaagatgatgtcgacctggcgcag

SEQ ID NO: 174 - DNA, 49 bp, Artificial Sequence
  Synthetic DNA
gatgtcgacctggcgcagatgtataaagactataacctgaacatcgccc

SEQ ID NO: 175 - DNA, 47 bp, Artificial Sequence
  Synthetic DNA
ctataacctgaacatcgcccttgatatggcccgctattgcatccacg

SEQ ID NO: 176 - DNA, 49 bp, Artificial Sequence
  Synthetic DNA
ccgctattgcatccacgacgcctgtctgtgccaatacctgtgggagtac

SEQ ID NO: 177 - DNA, 50 bp, Artificial Sequence
  Synthetic DNA
gtgccaatacctgtgggagtactatggtgtagagacgaaaacggatgcgg

SEQ ID NO: 178 - DNA, 46 bp, Artificial Sequence
  Synthetic DNA
gacgaaaacggatgcgggtgcctctacctatgtgttgcctcagtcc

SEQ ID NO: 179 - DNA, 49 bp, Artificial Sequence
  Synthetic DNA
ctatgtgttgcctcagtccatggtgtttgagtataaagcgagcacggtg

SEQ ID NO: 180 - DNA, 31 bp, Artificial Sequence
  Synthetic DNA
gtataaagcgagcacggtgattaaggggccc

SEQ ID NO: 181 - DNA, 29 bp, Artificial Sequence
  Synthetic DNA
cggtgattaaggggccctatggctgccgc

SEQ ID NO: 182 - DNA, 47 bp, Artificial Sequence
  Synthetic DNA
cggtgattaaggggccctacccatacgatgttccggattacgcttaa

SEQ ID NO: 183 - DNA, 30 bp, Artificial Sequence
  Synthetic DNA
tcctcgagcatagggcccctgctgaaattg

SEQ ID NO: 184 - DNA, 48 bp, Artificial Sequence
  Synthetic DNA
gacgacgacgacaagcatatgctcgaggatgggcccctgctgaaattg

SEQ ID NO: 185 - DNA, 50 bp, Artificial Sequence
  Synthetic DNA
gggcccctgctgaaattgctgctggaaaccaagaccatcttagtgcgctc

SEQ ID NO: 186 - DNA, 50 bp, Artificial Sequence
  Synthetic DNA
gaccatcttagtgcgctctgaaaccaaacaaaagttcccctatgaaggcg

SEQ ID NO: 187 - DNA, 46 bp, Artificial Sequence
  Synthetic DNA
gttcccctatgaaggcggtaaagtttttgccccgaagcagaagatg

SEQ ID NO: 188 - DNA, 50 bp, Artificial Sequence
  Synthetic DNA
ccccgaagcagaagatgtttagtaacaacgtcctgatctttgactacaac

SEQ ID NO: 189 - DNA, 50 bp, Artificial Sequence
  Synthetic DNA
cgtcctgatctttgactacaactctctgtatcccaacgtgtgcatctttg

SEQ ID NO: 190 - DNA, 50 bp, Artificial Sequence
  Synthetic DNA
ccaacgtgtgcatctttggcaacttaagtccggaaaccctggttggcgtg

SEQ ID NO: 191 - DNA, 48 bp, Artificial Sequence
  Synthetic DNA
gaaaccctggttggcgtggtggtgtcttcgaaccgcttggaagaagag

SEQ ID NO: 192 - DNA, 50 bp, Artificial Sequence
  Synthetic DNA
gaaccgcttggaagaagagattaacaaccagctgctcctgcaaaagtacc

SEQ ID NO: 193 - DNA, 49 bp, Artificial Sequence
  Synthetic DNA
ctgctcctgcaaaagtacccgccgccacgttacattacggtgcactgcg

SEQ ID NO: 194 - DNA, 50 bp, Artificial Sequence
  Synthetic DNA
cattacggtgcactgcgaaccacgtttacccaacctgatcagcgagattg

SEQ ID NO: 195 - DNA, 49 bp, Artificial Sequence
  Synthetic DNA
caacctgatcagcgagattgccatttttgatcggagcattgaaggcacc

SEQ ID NO: 196 - DNA, 50 bp, Artificial Sequence
  Synthetic DNA
ggagcattgaaggcaccattccgcgtttactgagaacctttctggccgag

SEQ ID NO: 197 - DNA, 50 bp, Artificial Sequence; identical to SEQ ID NO: 196
  Synthetic DNA

SEQ ID NO: 198 - DNA, 50 bp, Artificial Sequence
  Synthetic DNA
gaacctttctggccgagcgtgcgcgttataagaaaatgctgaaacaggcg

SEQ ID NO: 199 - DNA, 49 bp, Artificial Sequence
  Synthetic DNA
gaaaatgctgaaacaggcgaccagttctacggaaaaagccatctacgac

SEQ ID NO: 200 - DNA, 50 bp, Artificial Sequence
  Synthetic DNA
cggaaaaagccatctacgacagtatgcagtacacctacaagatcatcgcg

SEQ ID NO: 201 - DNA, 49 bp, Artificial Sequence
  Synthetic DNA
cacctacaagatcatcgcgaatagtgtgtatggcttgatgggttttcgc

SEQ ID NO: 202 - DNA, 50 bp, Artificial Sequence
  Synthetic DNA
gcttgatgggttttcgcaactctgccttgtatagctatgccagcgctaag

SEQ ID NO: 203 - DNA, 50 bp, Artificial Sequence
  Synthetic DNA
gctatgccagcgctaagagttgtaccagtattggccgtcgtatgatcctg

SEQ ID NO: 204 - DNA, 50 bp, Artificial Sequence
  Synthetic DNA
gccgtcgtatgatcctgtatctggaatctgtactcaatggagcggaactg

SEQ ID NO: 205 - DNA, 48 bp, Artificial Sequence
  Synthetic DNA
ctcaatggagcggaactgagtaatggcatgcttcgttttgcaaacccg

SEQ ID NO: 206 - DNA, 48 bp, Artificial Sequence
  Synthetic DNA
cttcgttttgcaaacccgttaagtaatccgttctacatggatgatcgc

SEQ ID NO: 207 - DNA, 48 bp, Artificial Sequence
  Synthetic DNA
cgttctacatggatgatcgcgacattaacccgattgtgaagacgtccc

SEQ ID NO: 208 - DNA, 47 bp, Artificial Sequence
  Synthetic DNA
cgattgtgaagacgtccctgccgattgactaccgttttcgcttcagg

SEQ ID NO: 209 - DNA, 47 bp, Artificial Sequence; identical to SEQ ID NO: 208
  Synthetic DNA

SEQ ID NO: 210 - DNA, 46 bp, Artificial Sequence
  Synthetic DNA
ctaccgttttcgcttcaggagtgtctatggtgataccgactccgtg

SEQ ID NO: 211 - DNA, 47 bp, Artificial Sequence
  Synthetic DNA
gtgataccgactccgtgtttaccgaaattgacagccaggatgttgac

SEQ ID NO: 212 - DNA, 46 bp, Artificial Sequence
  Synthetic DNA
gacagccaggatgttgacaaaagtattgagatagcgaaggagctgg

SEQ ID NO: 213 - DNA, 46 bp, Artificial Sequence
  Synthetic DNA
gatagcgaaggagctggaacgtctgatcaactctcgtgtgctgttc

SEQ ID NO: 214 - DNA, 47 bp, Artificial Sequence
  Synthetic DNA
caactctcgtgtgctgttcaacaactttaagatcgagtttgaggccg

SEQ ID NO: 215 - DNA, 46 bp, Artificial Sequence
  Synthetic DNA
gatcgagtttgaggccgtgtataaaaacctgatcatgcagagcaag

SEQ ID NO: 216 - DNA, 49 bp, Artificial Sequence
  Synthetic DNA
cctgatcatgcagagcaagaaaaaatataccacgatgaagtatagcgcg

SEQ ID NO: 217 - DNA, 47 bp, Artificial Sequence
  Synthetic DNA
cacgatgaagtatagcgcgagttctaactccaaaagtgtgccggagc

SEQ ID NO: 218 - DNA, 44 bp, Artificial Sequence
  Synthetic DNA
caaaagtgtgccggagcgtattaacaaggggactagcgaaaccc

SEQ ID NO: 219 - DNA, 44 bp, Artificial Sequence; identical to SEQ ID NO: 218
  Synthetic DNA

SEQ ID NO: 220 - DNA, 49 bp, Artificial Sequence
  Synthetic DNA
ggggactagcgaaacccgtcgtgatgtcagcaagttccacaaaaacatg

SEQ ID NO: 221 - DNA, 50 bp, Artificial Sequence
  Synthetic DNA
cagcaagttccacaaaaacatgattaaaatttacaagacccgtttgagcg

SEQ ID NO: 222 - DNA, 50 bp, Artificial Sequence
  Synthetic DNA
caagacccgtttgagcgaaatgttaagtgaaggccggatgaacagcaacc

SEQ ID NO: 223 - DNA, 44 bp, Artificial Sequence
  Synthetic DNA
ccggatgaacagcaaccaggtgtgtatcgacattctgcgttccc

SEQ ID NO: 224 - DNA, 46 bp, Artificial Sequence
  Synthetic DNA
cgacattctgcgttcccttgaaacggatcttcgtagcgagttcgac

SEQ ID NO: 225 - DNA, 45 bp, Artificial Sequence
  Synthetic DNA
cttcgtagcgagttcgacagccgatctagcccgttggaactgttc

SEQ ID NO: 226 - DNA, 44 bp, Artificial Sequence
  Synthetic DNA
gcccgttggaactgttcatgttaagccgcatgcaccacttgaac

SEQ ID NO: 227 - DNA, 48 bp, Artificial Sequence
  Synthetic DNA
gcatgcaccacttgaactataaaagcgccgataacccgaacatgtacc

SEQ ID NO: 228 - DNA, 50 bp, Artificial Sequence
  Synthetic DNA
cgataacccgaacatgtacctggtgaccgagtacaacaaaaacaacccgg

SEQ ID NO: 229 - DNA, 50 bp, Artificial Sequence; identical to SEQ ID NO: 228
  Synthetic DNA

SEQ ID NO: 230 - DNA, 49 bp, Artificial Sequence
  Synthetic DNA
gtacaacaaaaacaacccggaaactattgaacttggcgaacgctactac

SEQ ID NO: 231 - DNA, 50 bp, Artificial Sequence
  Synthetic DNA
cttggcgaacgctactactttgcctatatctgtccggcgaatgttccgtg

SEQ ID NO: 232 - DNA, 49 bp, Artificial Sequence
  Synthetic DNA
ccggcgaatgttccgtggaccaaaaaactcgtgaacatcaagacgtacg

SEQ ID NO: 233 - DNA, 50 bp, Artificial Sequence
  Synthetic DNA
cgtgaacatcaagacgtacgaaaccattattgaccgttccttcaagctgg

SEQ ID NO: 234 - DNA, 46 bp, Artificial Sequence
  Synthetic DNA
ccgttccttcaagctgggctcagatcagcgcattttttacgaggtg

SEQ ID NO: 235 - DNA, 49 bp, Artificial Sequence
  Synthetic DNA
gcgcattttttacgaggtgtattttaaacgtctgacctccgaaatcgtg

SEQ ID NO: 236 - DNA, 50 bp, Artificial Sequence
  Synthetic DNA
ctgacctccgaaatcgtgaacctgttagataacaaggtgctgtgcatttc

SEQ ID NO: 237 - DNA, 49 bp, Artificial Sequence
  Synthetic DNA
caaggtgctgtgcatttctttttttgaacgcatgtttggcagcagaccg

SEQ ID NO: 238 - DNA, 34 bp, Artificial Sequence
  Synthetic DNA
catgtttggcagcagaccgaccttctatgaggcg

SEQ ID NO: 239 - DNA, 29 bp, Artificial Sequence
  Synthetic DNA
cgaccttctatgaggcgtatggctgccgc

SEQ ID NO: 240 - DNA, 47 bp, Artificial Sequence
  Synthetic DNA
cgaccttctatgaggcgtacccatacgatgttccggattacgcttaa

SEQ ID NO: 241 - DNA, 48 bp, Artificial Sequence; identical to SEQ ID NO: 127
  Synthetic DNA

SEQ ID NO: 242 - DNA, 50 bp, Artificial Sequence
  Synthetic DNA
cccattacgacagcggcttttcagatataaaaacctgttttcaccatgcg

SEQ ID NO: 243 - DNA, 47 bp, Artificial Sequence; identical to SEQ ID NO: 129
  Synthetic DNA

SEQ ID NO: 244 - DNA, 50 bp, Artificial Sequence
  Synthetic DNA
ggtagatctcatcggtcaccacatagtaaaagtagtgagggaagcgaatg

SEQ ID NO: 245 - DNA, 50 bp, Artificial Sequence; identical to SEQ ID NO: 131
  Synthetic DNA

SEQ ID NO: 246 - DNA, 50 bp, Artificial Sequence
  Synthetic DNA
cgatgtcaatggtccgcattttacccataggacgagcgttgaaaggtggg

SEQ ID NO: 247 - DNA, 50 bp, Artificial Sequence; identical to SEQ ID NO: 133
  Synthetic DNA

SEQ ID NO: 248 - DNA, 49 bp, Artificial Sequence
  Synthetic DNA
cacatgtccgccacagagcatttacgatccttgatgtccaggttgtacg

SEQ ID NO: 249 - DNA, 46 bp, Artificial Sequence; identical to SEQ ID NO: 135
  Synthetic DNA

SEQ ID NO: 250 - DNA, 48 bp, Artificial Sequence
  Synthetic DNA
cagaaactcatccatggtggcattctgaatgttgcgctttttcggctc

SEQ ID NO: 251 - DNA, 50 bp, Artificial Sequence; identical to SEQ ID NO: 137
  Synthetic DNA

SEQ ID NO: 252 - DNA, 50 bp, Artificial Sequence
  Synthetic DNA
gtccaagctgtagcatccatccggagaaatgccgttgctgatgtagaacc

SEQ ID NO: 253 - DNA, 50 bp, Artificial Sequence; identical to SEQ ID NO: 138
  Synthetic DNA

SEQ ID NO: 254 - DNA, 50 bp, Artificial Sequence
  Synthetic DNA
cacccgttgttgattttcgtgagatactgatcgtccaagctgtagcatcc

SEQ ID NO: 255 - DNA, 48 bp, Artificial Sequence; identical to SEQ ID NO: 141
  Synthetic DNA

SEQ ID NO: 256 - DNA, 45 bp, Artificial Sequence
  Synthetic DNA
gtcaaaacgggggatctctttcgcaaaacagttacgagggtcgcc

SEQ ID NO: 257 - DNA, 49 bp, Artificial Sequence; identical to SEQ ID NO: 143
  Synthetic DNA

SEQ ID NO: 258 - DNA, 50 bp, Artificial Sequence
  Synthetic DNA
gctcgggaacttcttatcgaaatggcattcaatgtccaggaacagatagc

SEQ ID NO: 259 - DNA, 49 bp, Artificial Sequence; identical to SEQ ID NO: 145
  Synthetic DNA

SEQ ID NO: 260 - DNA, 50 bp, Artificial Sequence
  Synthetic DNA
cagacgtttgccgctcagatcaatataacaataggaggtatggctgatcg

SEQ ID NO: 261 - DNA, 50 bp, Artificial Sequence; identical to SEQ ID NO: 147
  Synthetic DNA

SEQ ID NO: 262 - DNA, 50 bp, Artificial Sequence
  Synthetic DNA
gatccacggcctcctggatttcttgttcggtcagcatctcctcgttgatc

SEQ ID NO: 263 - DNA, 49 bp, Artificial Sequence; identical to SEQ ID NO: 149
  Synthetic DNA

SEQ ID NO: 264 - DNA, 50 bp, Artificial Sequence
  Synthetic DNA
gcacagcaccagttcacgttcataatccatctccatcaaggactgaatgc

SEQ ID NO: 265 - DNA, 50 bp, Artificial Sequence; identical to SEQ ID NO: 150
  Synthetic DNA

SEQ ID NO: 266 - DNA, 48 bp, Artificial Sequence
  Synthetic DNA
gtttggcgatttggagcagcacaatttcagagcacagcaccagttcac

SEQ ID NO: 267 - DNA, 49 bp, Artificial Sequence; identical to SEQ ID NO: 153
  Synthetic DNA

SEQ ID NO: 268 - DNA, 50 bp, Artificial Sequence
  Synthetic DNA
gcagatcgaagttgtggccgttgaacgtcacgatgtaatcaaaggtcagc

SEQ ID NO: 269 - DNA, 50 bp, Artificial Sequence; identical to SEQ ID NO: 155
  Synthetic DNA

SEQ ID NO: 270 - DNA, 50 bp, Artificial Sequence
  Synthetic DNA
cttgtcggggctacgaaagatgattttttcgccggtcaacagctcgagac

SEQ ID NO: 271 - DNA, 50 bp, Artificial Sequence; identical to SEQ ID NO: 157
  Synthetic DNA

SEQ ID NO: 272 - DNA, 46 bp, Artificial Sequence
  Synthetic DNA
caacccctttgtggctcgactgattacgctcatagatgcacaggtg

SEQ ID NO: 273 - DNA, 46 bp, Artificial Sequence; identical to SEQ ID NO: 159
  Synthetic DNA

SEQ ID NO: 274 - DNA, 50 bp, Artificial Sequence
  Synthetic DNA
ggtcgaaaaaaatggtgccattattattattgacgtggaaggtcgtattc

SEQ ID NO: 275 - DNA, 50 bp, Artificial Sequence; identical to SEQ ID NO: 160
  Synthetic DNA

SEQ ID NO: 276 - DNA, 49 bp, Artificial Sequence
  Synthetic DNA
gcttctccgatttctggatgaaagaatacaggtcgaaaaaaatggtgcc

SEQ ID NO: 277 - DNA, 49 bp, Artificial Sequence; identical to SEQ ID NO: 163
  Synthetic DNA

SEQ ID NO: 278 - DNA, 50 bp, Artificial Sequence
  Synthetic DNA
gcactttgcccatgcacgaaaaggcgtttttgctgatgctgtccagtttg

SEQ ID NO: 279 - DNA, 49 bp, Artificial Sequence; identical to SEQ ID NO: 165
  Synthetic DNA

SEQ ID NO: 280 - DNA, 48 bp, Artificial Sequence
  Synthetic DNA
ccctttcgcatcagtggtatcatcaccgataaaggtcatctcacgcac

SEQ ID NO: 281 - DNA, 47 bp, Artificial Sequence; identical to SEQ ID NO: 167
  Synthetic DNA

SEQ ID NO: 282 - DNA, 49 bp, Artificial Sequence
  Synthetic DNA
gatatcatcgaccgtcacgtaattgcctgtggtgaggaccttcgcaaac

SEQ ID NO: 283 - DNA, 50 bp, Artificial Sequence; identical to SEQ ID NO: 169
  Synthetic DNA

SEQ ID NO: 284 - DNA, 50 bp, Artificial Sequence
  Synthetic DNA
gacagctcaacaccaccttaaagccattttcccagatgtccttgtgaatc

SEQ ID NO: 285 - DNA, 50 bp, Artificial Sequence; identical to SEQ ID NO: 170
  Synthetic DNA

SEQ ID NO: 286 - DNA, 49 bp, Artificial Sequence
  Synthetic DNA
ggagagtttgtacgtgtcgttggtcagagtcggacagctcaacaccacc

SEQ ID NO: 287 - DNA, 49 bp, Artificial Sequence; identical to SEQ ID NO: 173
  Synthetic DNA

SEQ ID NO: 288 - DNA, 49 bp, Artificial Sequence
  Synthetic DNA
gggcgatgttcaggttatagtctttatacatctgcgccaggtcgacatc

SEQ ID NO: 289 - DNA, 47 bp, Artificial Sequence; identical to SEQ ID NO: 175
  Synthetic DNA

SEQ ID NO: 290 - DNA, 49 bp, Artificial Sequence
  Synthetic DNA
gtactcccacaggtattggcacagacaggcgtcgtggatgcaatagcgg

SEQ ID NO: 291 - DNA, 50 bp, Artificial Sequence; identical to SEQ ID NO: 177
  Synthetic DNA

SEQ ID NO: 292 - DNA, 46 bp, Artificial Sequence
  Synthetic DNA
ggactgaggcaacacataggtagaggcacccgcatccgttttcgtc

SEQ ID NO: 293 - DNA, 49 bp, Artificial Sequence; identical to SEQ ID NO: 179
  Synthetic DNA

SEQ ID NO: 294 - DNA, 31 bp, Artificial Sequence
  Synthetic DNA
gggccccttaatcaccgtgctcgctttatac

SEQ ID NO: 295 - DNA, 50 bp, Artificial Sequence; identical to SEQ ID NO: 185
  Synthetic DNA

SEQ ID NO: 296 - DNA, 50 bp, Artificial Sequence
  Synthetic DNA
cgccttcataggggaacttttgtttggtttcagagcgcactaagatggtc

SEQ ID NO: 297 - DNA, 46 bp, Artificial Sequence; identical to SEQ ID NO: 187
  Synthetic DNA

SEQ ID NO: 298 - DNA, 50 bp, Artificial Sequence
  Synthetic DNA
gttgtagtcaaagatcaggacgttgttactaaacatcttctgcttcgggg

SEQ ID NO: 299 - DNA, 50 bp, Artificial Sequence; identical to SEQ ID NO: 189
  Synthetic DNA

SEQ ID NO: 300 - DNA, 50 bp, Artificial Sequence
  Synthetic DNA
cacgccaaccagggtttccggacttaagttgccaaagatgcacacgttgg

SEQ ID NO: 301 - DNA, 48 bp, Artificial Sequence; identical to SEQ ID NO: 191
  Synthetic DNA

SEQ ID NO: 302 - DNA, 50 bp, Artificial Sequence
  Synthetic DNA
ggtacttttgcaggagcagctggttgttaatctcttcttccaagcggttc

SEQ ID NO: 303 - DNA, 49 bp, Artificial Sequence; identical to SEQ ID NO: 193
  Synthetic DNA

SEQ ID NO: 304 - DNA, 50 bp, Artificial Sequence
  Synthetic DNA
caatctcgctgatcaggttgggtaaacgtggttcgcagtgcaccgtaatg

SEQ ID NO: 305 - DNA, 49 bp, Artificial Sequence; identical to SEQ ID NO: 195
  Synthetic DNA

SEQ ID NO: 306 - DNA, 50 bp, Artificial Sequence
  Synthetic DNA
ctcggccagaaaggttctcagtaaacgcggaatggtgccttcaatgctcc

SEQ ID NO: 307 - DNA, 50 bp, Artificial Sequence; identical to SEQ ID NO: 196
  Synthetic DNA

SEQ ID NO: 308 - DNA, 50 bp, Artificial Sequence
  Synthetic DNA
cgcctgtttcagcattttcttataacgcgcacgctcggccagaaaggttc

SEQ ID NO: 309 - DNA, 49 bp, Artificial Sequence; identical to SEQ ID NO: 199
  Synthetic DNA

SEQ ID NO: 310 - DNA, 50 bp, Artificial Sequence
  Synthetic DNA
cgcgatgatcttgtaggtgtactgcatactgtcgtagatggctttttccg

SEQ ID NO: 311 - DNA, 49 bp, Artificial Sequence; identical to SEQ ID NO: 201
  Synthetic DNA

SEQ ID NO: 312 - DNA, 50 bp, Artificial Sequence
  Synthetic DNA
cttagcgctggcatagctatacaaggcagagttgcgaaaacccatcaagc

SEQ ID NO: 313 - DNA, 50 bp, Artificial Sequence; identical to SEQ ID NO: 203
  Synthetic DNA

SEQ ID NO: 314 - DNA, 50 bp, Artificial Sequence
  Synthetic DNA
cagttccgctccattgagtacagattccagatacaggatcatacgacggc

SEQ ID NO: 315 - DNA, 48 bp, Artificial Sequence; identical to SEQ ID NO: 205
  Synthetic DNA

SEQ ID NO: 316 - DNA, 48 bp, Artificial Sequence
  Synthetic DNA
gcgatcatccatgtagaacggattacttaacgggtttgcaaaacgaag

SEQ ID NO: 317 - DNA, 48 bp, Artificial Sequence; identical to SEQ ID NO: 207
  Synthetic DNA

SEQ ID NO: 318 - DNA, 47 bp, Artificial Sequence
  Synthetic DNA
cctgaagcgaaaacggtagtcaatcggcagggacgtcttcacaatcg

SEQ ID NO: 319 - DNA, 47 bp, Artificial Sequence; identical to SEQ ID NO: 208
  Synthetic DNA

SEQ ID NO: 320 - DNA, 46 bp, Artificial Sequence
  Synthetic DNA
cacggagtcggtatcaccatagacactcctgaagcgaaaacggtag

SEQ ID NO: 321 - DNA, 47 bp, Artificial Sequence; identical to SEQ ID NO: 211
  Synthetic DNA

SEQ ID NO: 322 - DNA, 46 bp, Artificial Sequence
  Synthetic DNA
ccagctccttcgctatctcaatacttttgtcaacatcctggctgtc

SEQ ID NO: 323 - DNA, 46 bp, Artificial Sequence; identical to SEQ ID NO: 213
  Synthetic DNA

SEQ ID NO: 324 - DNA, 47 bp, Artificial Sequence
  Synthetic DNA
cggcctcaaactcgatcttaaagttgttgaacagcacacgagagttg

SEQ ID NO: 325 - DNA, 46 bp, Artificial Sequence; identical to SEQ ID NO: 215
  Synthetic DNA

SEQ ID NO: 326 - DNA, 49 bp, Artificial Sequence
  Synthetic DNA
cgcgctatacttcatcgtggtatattttttcttgctctgcatgatcagg

SEQ ID NO: 327 - DNA, 47 bp, Artificial Sequence; identical to SEQ ID NO: 217
  Synthetic DNA

SEQ ID NO: 328 - DNA, 44 bp, Artificial Sequence
  Synthetic DNA
gggtttcgctagtccccttgttaatacgctccggcacacttttg

SEQ ID NO: 329 - DNA, 44 bp, Artificial Sequence; identical to SEQ ID NO: 218
  Synthetic DNA

SEQ ID NO: 330 - DNA, 49 bp, Artificial Sequence
  Synthetic DNA
catgtttttgtggaacttgctgacatcacgacgggtttcgctagtcccc

SEQ ID NO: 331 - DNA, 50 bp, Artificial Sequence; identical to SEQ ID NO: 221
  Synthetic DNA

SEQ ID NO: 332 - DNA, 50 bp, Artificial Sequence
  Synthetic DNA
ggttgctgttcatccggccttcacttaacatttcgctcaaacgggtcttg

SEQ ID NO: 333 - DNA, 44 bp, Artificial Sequence; identical to SEQ ID NO: 223
  Synthetic DNA

SEQ ID NO: 334 - DNA, 46 bp, Artificial Sequence
  Synthetic DNA
gtcgaactcgctacgaagatccgtttcaagggaacgcagaatgtcg

SEQ ID NO: 335 - DNA, 45 bp, Artificial Sequence; identical to SEQ ID NO: 225
  Synthetic DNA

SEQ ID NO: 336 - DNA, 44 bp, Artificial Sequence
  Synthetic DNA
gttcaagtggtgcatgcggcttaacatgaacagttccaacgggc

SEQ ID NO: 337 - DNA, 48 bp, Artificial Sequence; identical to SEQ ID NO: 227
  Synthetic DNA

SEQ ID NO: 338 - DNA, 50 bp, Artificial Sequence
  Synthetic DNA
ccgggttgtttttgttgtactcggtcaccaggtacatgttcgggttatcg

SEQ ID NO: 339 - DNA, 50 bp, Artificial Sequence; identical to SEQ ID NO: 228
  Synthetic DNA

SEQ ID NO: 340 - DNA, 49 bp, Artificial Sequence
  Synthetic DNA
gtagtagcgttcgccaagttcaatagtttccgggttgtttttgttgtac

SEQ ID NO: 341 - DNA, 50 bp, Artificial Sequence; identical to SEQ ID NO: 231
  Synthetic DNA

SEQ ID NO: 342 - DNA, 49 bp, Artificial Sequence
  Synthetic DNA
cgtacgtcttgatgttcacgagttttttggtccacggaacattcgccgg

SEQ ID NO: 343 - DNA, 50 bp, Artificial Sequence; identical to SEQ ID NO: 233
  Synthetic DNA

SEQ ID NO: 344 - DNA, 46 bp, Artificial Sequence
  Synthetic DNA
cacctcgtaaaaaatgcgctgatctgagcccagcttgaaggaacgg

SEQ ID NO: 345 - DNA, 49 bp, Artificial Sequence; identical to SEQ ID NO: 235
  Synthetic DNA

SEQ ID NO: 346 - DNA, 50 bp, Artificial Sequence
  Synthetic DNA
gaaatgcacagcaccttgttatctaacaggttcacgatttcggaggtcag

SEQ ID NO: 347 - DNA, 49 bp, Artificial Sequence; identical to SEQ ID NO: 237
  Synthetic DNA

SEQ ID NO: 348 - DNA, 34 bp, Artificial Sequence
  Synthetic DNA
cgcctcatagaaggtcggtctgctgccaaacatg

SEQ ID NO: 349 - DNA, 29 bp, Artificial Sequence
  Synthetic DNA
gcggcagccatagggccccttaatcaccg

SEQ ID NO: 350 - DNA, 47 bp, Artificial Sequence
  Synthetic DNA
ttaagcgtaatccggaacatcgtatgggtagggccccttaatcaccg

SEQ ID NO: 351 - DNA, 29 bp, Artificial Sequence
  Synthetic DNA
gcggcagccatacgcctcatagaaggtcg

SEQ ID NO: 352 - DNA, 47 bp, Artificial Sequence
  Synthetic DNA
ttaagcgtaatccggaacatcgtatgggtacgcctcatagaaggtcg

SEQ ID NO: 353 - DNA, 1542 bp, Artificial Sequence
  Synthetic DNA
atggcggattctcagccgttgtctggtgccccggaaggcgcggagtatcttcgggcggtactgcgagctccagtgtatgaagccgcacaggtgaccccgctgcaaaaaatggagaaactgagctcccgtctggataacgtcatcctggtcaaacgcgaagatcgtcagccggttcacagcttcaaactgcgcggggcctatgctatgatggcgggcttaacggaagagcagaaagcacatggcgttattaccgcgtctgcaggcaaccatgcacagggggtagcgttttctagtgcgcgtttaggcgtgaaagcgttgatcgtgatgccgaccgcaaccgctgatatcaaagtcgatgccgtgcgtggctttggcggagaagtgctgttacacggcgcaaacttcgatgaagcaaaagccaaagcgatcgaactgtctcagcagcaggggtttacctgggttccgccgttcgatcatccgatggtgattgctggtcagggcactctggcgttagaactgctccagcaagatgcccacctggatcgtgtgtttgtgccggtaggtggaggaggccttgctgcaggagtcgcagtgctgatcaaacagttgatgccgcagatcaaggttattgccgtggaagccgaggatagcgcctgtctgaaagcggcgttagatgctggtcatccggtcgatctgccacgtgtaggcctgtttgcggaaggtgtagcggtgaagcgtattggcgacgaaacctttcggctgtgccaggaatatctcgacgacatcatcaccgttgacagcgatgcgatttgtgccgcgatgaaagacctgttcgaagatgtgcgtgcggtagctgaaccaagtggtgcattagcgttggccggcatgaaaaagtacattgccttgcacaacattcgtggcgaacgcctggcccatatcctgagtggagccaacgtcaacttccatggcctgcgttacgttagcgaacggtgtgaactgggcgagcaacgtgaagcgctcctggccgttaccatcccagaggagaagggcagctttctgaaattttgccagctgttagggggccgtagcgtcaccgaattcaactaccgctttgccgatgcgaaaaatgcgtgcattttcgtgggcgtccgtctgtcccgtggcctggaagagcgcaaggagattctgcagatgctgaacgatggtggttattccgtggtggatctcagcgacgatgaaatggccaagctgcatgtgcgctatatggtggggggtcgtccgagtcacccgttgcaggaacgcttgtacagcttcgagtttccggagtctcctggtgcactgttacgcttcctgaacaccctggggacgtactggaacatcagcctgtttcactatcgctcccatggtactgactacggtcgggtcctggctgcgtttgaactgggggaccacgaaccggacttcgaaacccgcctgaacgaattaggctacgactgccatgacgaaaccaacaacccggcgtttcgcttttttctggcaggg

SEQ ID NO: 354 - DNA, 33 bp, Artificial Sequence
  Synthetic DNA
atggcggattctcagccgttgtctggtgccccg

SEQ ID NO: 355 - DNA, 60 bp, Artificial Sequence
  Synthetic DNA
atggcggattctcagccgttgtctggtgccccggaaggcgcggagtatcttcgggcggta

SEQ ID NO: 356 - DNA, 56 bp, Artificial Sequence
  Synthetic DNA
gaaggcgcggagtatcttcgggcggtactgcgagctccagtgtatgaagccgcaca

SEQ ID NO: 357 - DNA, 60 bp, Artificial Sequence
  Synthetic DNA
ctgcgagctccagtgtatgaagccgcacaggtgaccccgctgcaaaaaatggagaaactg

SEQ ID NO: 358 - DNA, 60 bp, Artificial Sequence
  Synthetic DNA
ggtgaccccgctgcaaaaaatggagaaactgagctcccgtctggataacgtcatcctggt

SEQ ID NO: 359 - DNA, 60 bp, Artificial Sequence
  Synthetic DNA
agctcccgtctggataacgtcatcctggtcaaacgcgaagatcgtcagccggttcacagc

SEQ ID NO: 360 - DNA, 60 bp, Artificial Sequence
  Synthetic DNA
caaacgcgaagatcgtcagccggttcacagcttcaaactgcgcggggcctatgctatgat

SEQ ID NO: 361 - DNA, 60 bp, Artificial Sequence
  Synthetic DNA
ttcaaactgcgcggggcctatgctatgatggcgggcttaacggaagagcagaaagcacat

SEQ ID NO: 362 - DNA, 60 bp, Artificial Sequence
  Synthetic DNA
ggcgggcttaacggaagagcagaaagcacatggcgttattaccgcgtctgcaggcaacca

SEQ ID NO: 363 - DNA, 60 bp, Artificial Sequence
  Synthetic DNA
ggcgttattaccgcgtctgcaggcaaccatgcacagggggtagcgttttctagtgcgcgt

SEQ ID NO: 364 - DNA, 60 bp, Artificial Sequence
  Synthetic DNA
tgcacagggggtagcgttttctagtgcgcgtttaggcgtgaaagcgttgatcgtgatgcc

SEQ ID NO: 365 - DNA, 60 bp, Artificial Sequence
  Synthetic DNA
ttaggcgtgaaagcgttgatcgtgatgccgaccgcaaccgctgatatcaaagtcgatgcc

SEQ ID NO: 366 - DNA, 60 bp, Artificial Sequence
  Synthetic DNA
gaccgcaaccgctgatatcaaagtcgatgccgtgcgtggctttggcggagaagtgctgtt

SEQ ID NO: 367 - DNA, 60 bp, Artificial Sequence
  Synthetic DNA
gtgcgtggctttggcggagaagtgctgttacacggcgcaaacttcgatgaagcaaaagcc

SEQ ID NO: 368 - DNA, 60 bp, Artificial Sequence
  Synthetic DNA
acacggcgcaaacttcgatgaagcaaaagccaaagcgatcgaactgtctcagcagcaggg

SEQ ID NO: 369 - DNA, 60 bp, Artificial Sequence
  Synthetic DNA
aaagcgatcgaactgtctcagcagcaggggtttacctgggttccgccgttcgatcatccg

SEQ ID NO: 370 - DNA, 60 bp, Artificial Sequence
  Synthetic DNA
gtttacctgggttccgccgttcgatcatccgatggtgattgctggtcagggcactctggc

SEQ ID NO: 371 - DNA, 58 bp, Artificial Sequence
  Synthetic DNA
atggtgattgctggtcagggcactctggcgttagaactgctccagcaagatgcccacc

SEQ ID NO: 372 - DNA, 56 bp, Artificial Sequence
  Synthetic DNA
gttagaactgctccagcaagatgcccacctggatcgtgtgtttgtgccggtaggtg

SEQ ID NO: 373 - DNA, 54 bp, Artificial Sequence
  Synthetic DNA
tggatcgtgtgtttgtgccggtaggtggaggaggccttgctgcaggagtcgcag

SEQ ID NO: 374 - DNA, 60 bp, Artificial Sequence
  Synthetic DNA
gaggaggccttgctgcaggagtcgcagtgctgatcaaacagttgatgccgcagatcaagg

SEQ ID NO: 375 - DNA, 60 bp, Artificial Sequence
  Synthetic DNA
tgctgatcaaacagttgatgccgcagatcaaggttattgccgtggaagccgaggatagcg

SEQ ID NO: 376 - DNA, 60 bp, Artificial Sequence
  Synthetic DNA
ttattgccgtggaagccgaggatagcgcctgtctgaaagcggcgttagatgctggtcatc

SEQ ID NO: 377 - DNA, 60 bp, Artificial Sequence
  Synthetic DNA
cctgtctgaaagcggcgttagatgctggtcatccggtcgatctgccacgtgtaggcctgt

SEQ ID NO: 378 - DNA, 60 bp, Artificial Sequence
  Synthetic DNA
cggtcgatctgccacgtgtaggcctgtttgcggaaggtgtagcggtgaagcgtattggcg

SEQ ID NO: 379 - DNA, 60 bp, Artificial Sequence
  Synthetic DNA
ttgcggaaggtgtagcggtgaagcgtattggcgacgaaacctttcggctgtgccaggaat

SEQ ID NO: 380 - DNA, 59 bp, Artificial Sequence
  Synthetic DNA
acgaaacctttcggctgtgccaggaatatctcgacgacatcatcaccgttgacagcgat

SEQ ID NO: 381 - DNA, 60 bp, Artificial Sequence
  Synthetic DNA
atctcgacgacatcatcaccgttgacagcgatgcgatttgtgccgcgatgaaagacctgt

SEQ ID NO: 382 - DNA, 60 bp, Artificial Sequence
  Synthetic DNA
gcgatttgtgccgcgatgaaagacctgttcgaagatgtgcgtgcggtagctgaaccaagt

SEQ ID NO: 383 - DNA, 60 bp, Artificial Sequence
  Synthetic DNA
tcgaagatgtgcgtgcggtagctgaaccaagtggtgcattagcgttggccggcatgaaaa

SEQ ID NO: 384 - DNA, 60 bp, Artificial Sequence
  Synthetic DNA
ggtgcattagcgttggccggcatgaaaaagtacattgccttgcacaacattcgtggcgaa

SEQ ID NO: 385 - DNA, 60 bp, Artificial Sequence
  Synthetic DNA
agtacattgccttgcacaacattcgtggcgaacgcctggcccatatcctgagtggagcca

SEQ ID NO: 386 - DNA, 55 bp, Artificial Sequence
  Synthetic DNA
cgcctggcccatatcctgagtggagccaacgtcaacttccatggcctgcgttacg

SEQ ID NO: 387 - DNA, 54 bp, Artificial Sequence
  Synthetic DNA
acgtcaacttccatggcctgcgttacgttagcgaacggtgtgaactgggcgagc

SEQ ID NO: 388 - DNA, 54 bp, Artificial Sequence
  Synthetic DNA
ttagcgaacggtgtgaactgggcgagcaacgtgaagcgctcctggccgttacca

SEQ ID NO: 389 - DNA, 54 bp, Artificial Sequence
  Synthetic DNA
aacgtgaagcgctcctggccgttaccatcccagaggagaagggcagctttctga

SEQ ID NO: 390 - DNA, 54 bp, Artificial Sequence
  Synthetic DNA
tcccagaggagaagggcagctttctgaaattttgccagctgttagggggccgta

SEQ ID NO: 391 - DNA, 59 bp, Artificial Sequence
  Synthetic DNA
aattttgccagctgttagggggccgtagcgtcaccgaattcaactaccgctttgccgat

SEQ ID NO: 392 - DNA, 60 bp, Artificial Sequence
  Synthetic DNA
gcgtcaccgaattcaactaccgctttgccgatgcgaaaaatgcgtgcattttcgtgggcg

SEQ ID NO: 393 - DNA, 55 bp, Artificial Sequence
  Synthetic DNA
gcgaaaaatgcgtgcattttcgtgggcgtccgtctgtcccgtggcctggaagagc

SEQ ID NO: 394 - DNA, 59 bp, Artificial Sequence
  Synthetic DNA
tccgtctgtcccgtggcctggaagagcgcaaggagattctgcagatgctgaacgatggt

SEQ ID NO: 395 - DNA, 60 bp, Artificial Sequence
  Synthetic DNA
gcaaggagattctgcagatgctgaacgatggtggttattccgtggtggatctcagcgacg

SEQ ID NO: 396 - DNA, 55 bp, Artificial Sequence
  Synthetic DNA
ggttattccgtggtggatctcagcgacgatgaaatggccaagctgcatgtgcgct

SEQ ID NO: 397 - DNA, 54 bp, Artificial Sequence
  Synthetic DNA
atgaaatggccaagctgcatgtgcgctatatggtggggggtcgtccgagtcacc

SEQ ID NO: 398 - DNA, 55 bp, Artificial Sequence
  Synthetic DNA
atatggtggggggtcgtccgagtcacccgttgcaggaacgcttgtacagcttcga

SEQ ID NO: 399 - DNA, 55 bp, Artificial Sequence
  Synthetic DNA
cgttgcaggaacgcttgtacagcttcgagtttccggagtctcctggtgcactgtt

SEQ ID NO: 400 - DNA, 54 bp, Artificial Sequence
  Synthetic DNA
gtttccggagtctcctggtgcactgttacgcttcctgaacaccctggggacgta

SEQ ID NO: 401 - DNA, 59 bp, Artificial Sequence
  Synthetic DNA
acgcttcctgaacaccctggggacgtactggaacatcagcctgtttcactatcgctccc

SEQ ID NO: 402 - DNA, 59 bp, Artificial Sequence
  Synthetic DNA
ctggaacatcagcctgtttcactatcgctcccatggtactgactacggtcgggtcctgg

SEQ ID NO: 403 - DNA, 54 bp, Artificial Sequence
  Synthetic DNA
atggtactgactacggtcgggtcctggctgcgtttgaactgggggaccacgaac

SEQ ID NO: 404 - DNA, 55 bp, Artificial Sequence
  Synthetic DNA
ctgcgtttgaactgggggaccacgaaccggacttcgaaacccgcctgaacgaatt

SEQ ID NO: 405 - DNA, 59 bp, Artificial Sequence
  Synthetic DNA
cggacttcgaaacccgcctgaacgaattaggctacgactgccatgacgaaaccaacaac

SEQ ID NO: 406 - DNA, 58 bp, Artificial Sequence
  Synthetic DNA
aggctacgactgccatgacgaaaccaacaacccggcgtttcgcttttttctggcaggg

SEQ ID NO: 407 - DNA, 27 bp, Artificial Sequence
  Synthetic DNA
ccggcgtttcgcttttttctggcaggg

SEQ ID NO: 408 - DNA, 45 bp, Artificial Sequence
  Synthetic DNA
tcctcgagcataatggcggattctcagccgttgtctggtgccccg

SEQ ID NO: 409 - DNA, 12 bp, Artificial Sequence
  Synthetic DNA
tatgctcgagga

SEQ ID NO: 410 - DNA, 63 bp, Artificial Sequence
  Synthetic DNA
gacgacgacgacaagcatatgctcgaggatatggcggattctcagccgttgtctggtgccccg

SEQ ID NO: 411 - DNA, 30 bp, Artificial Sequence
  Synthetic DNA
atcctcgagcatatgcttgtcgtcgtcgtc

SEQ ID NO: 412 - DNA, 33 bp, Artificial Sequence; identical to SEQ ID NO: 354
  Synthetic DNA

SEQ ID NO: 413 - DNA, 60 bp, Artificial Sequence
  Synthetic DNA
taccgcccgaagatactccgcgccttccggggcaccagacaacggctgagaatccgccat

SEQ ID NO: 414 - DNA, 56 bp, Artificial Sequence; identical to SEQ ID NO: 356
  Synthetic DNA

SEQ ID NO: 415 - DNA, 60 bp, Artificial Sequence
  Synthetic DNA
cagtttctccattttttgcagcggggtcacctgtgcggcttcatacactggagctcgcag

SEQ ID NO: 416 - DNA, 60 bp, Artificial Sequence; identical to SEQ ID NO: 358
  Synthetic DNA

SEQ ID NO: 417 - DNA, 60 bp, Artificial Sequence
  Synthetic DNA
gctgtgaaccggctgacgatcttcgcgtttgaccaggatgacgttatccagacgggagct

SEQ ID NO: 418 - DNA, 60 bp, Artificial Sequence; identical to SEQ ID NO: 360
  Synthetic DNA

SEQ ID NO: 419 - DNA, 60 bp, Artificial Sequence
  Synthetic DNA
atgtgctttctgctcttccgttaagcccgccatcatagcataggccccgcgcagtttgaa

SEQ ID NO: 420 - DNA, 60 bp, Artificial Sequence; identical to SEQ ID NO: 362
  Synthetic DNA

SEQ ID NO: 421 - DNA, 60 bp, Artificial Sequence
  Synthetic DNA
acgcgcactagaaaacgctaccccctgtgcatggttgcctgcagacgcggtaataacgcc

SEQ ID NO: 422 - DNA, 60 bp, Artificial Sequence; identical to SEQ ID NO: 364
  Synthetic DNA

SEQ ID NO: 423 - DNA, 60 bp, Artificial Sequence
  Synthetic DNA
ggcatcgactttgatatcagcggttgcggtcggcatcacgatcaacgctttcacgcctaa

SEQ ID NO: 424 - DNA, 60 bp, Artificial Sequence; identical to SEQ ID NO: 366
  Synthetic DNA

SEQ ID NO: 425 - DNA, 60 bp, Artificial Sequence
  Synthetic DNA
ggcttttgcttcatcgaagtttgcgccgtgtaacagcacttctccgccaaagccacgcac

SEQ ID NO: 426 - DNA, 60 bp, Artificial Sequence; identical to SEQ ID NO: 368
  Synthetic DNA

SEQ ID NO: 427 - DNA, 60 bp, Artificial Sequence
  Synthetic DNA
cggatgatcgaacggcggaacccaggtaaacccctgctgctgagacagttcgatcgcttt

SEQ ID NO: 428 - DNA, 60 bp, Artificial Sequence; identical to SEQ ID NO: 370
  Synthetic DNA

SEQ ID NO: 429 - DNA, 58 bp, Artificial Sequence
  Synthetic DNA
ggtgggcatcttgctggagcagttctaacgccagagtgccctgaccagcaatcaccat

SEQ ID NO: 430 - DNA, 56 bp, Artificial Sequence; identical to SEQ ID NO: 372
  Synthetic DNA

SEQ ID NO: 431 - DNA, 54 bp, Artificial Sequence
  Synthetic DNA
ctgcgactcctgcagcaaggcctcctccacctaccggcacaaacacacgatcca

SEQ ID NO: 432 - DNA, 60 bp, Artificial Sequence; identical to SEQ ID NO: 374
  Synthetic DNA

SEQ ID NO: 433 - DNA, 60 bp, Artificial Sequence
  Synthetic DNA
cgctatcctcggcttccacggcaataaccttgatctgcggcatcaactgtttgatcagca

SEQ ID NO: 434 - DNA, 60 bp, Artificial Sequence; identical to SEQ ID NO: 376
  Synthetic DNA

SEQ ID NO: 435 - DNA, 60 bp, Artificial Sequence
  Synthetic DNA
acaggcctacacgtggcagatcgaccggatgaccagcatctaacgccgctttcagacagg

SEQ ID NO: 436 - DNA, 60 bp, Artificial Sequence; identical to SEQ ID NO: 378
  Synthetic DNA

SEQ ID NO: 437 - DNA, 60 bp, Artificial Sequence
  Synthetic DNA
attcctggcacagccgaaaggtttcgtcgccaatacgcttcaccgctacaccttccgcaa

SEQ ID NO: 438 - DNA, 59 bp, Artificial Sequence; identical to SEQ ID NO: 380
  Synthetic DNA

SEQ ID NO: 439 - DNA, 60 bp, Artificial Sequence
  Synthetic DNA
acaggtctttcatcgcggcacaaatcgcatcgctgtcaacggtgatgatgtcgtcgagat

SEQ ID NO: 440 - DNA, 60 bp, Artificial Sequence; identical to SEQ ID NO: 382
  Synthetic DNA

SEQ ID NO: 441 - DNA, 60 bp, Artificial Sequence
  Synthetic DNA
ttttcatgccggccaacgctaatgcaccacttggttcagctaccgcacgcacatcttcga

SEQ ID NO: 442 - DNA, 60 bp, Artificial Sequence; identical to SEQ ID NO: 384
  Synthetic DNA

SEQ ID NO: 443 - DNA, 60 bp, Artificial Sequence
  Synthetic DNA
tggctccactcaggatatgggccaggcgttcgccacgaatgttgtgcaaggcaatgtact

SEQ ID NO: 444 - DNA, 55 bp, Artificial Sequence; identical to SEQ ID NO: 386
  Synthetic DNA

SEQ ID NO: 445 - DNA, 54 bp, Artificial Sequence
  Synthetic DNA
gctcgcccagttcacaccgttcgctaacgtaacgcaggccatggaagttgacgt

SEQ ID NO: 446 - DNA, 54 bp, Artificial Sequence; identical to SEQ ID NO: 388
  Synthetic DNA

SEQ ID NO: 447 - DNA, 54 bp, Artificial Sequence
  Synthetic DNA
tcagaaagctgcccttctcctctgggatggtaacggccaggagcgcttcacgtt

SEQ ID NO: 448 - DNA, 54 bp, Artificial Sequence; identical to SEQ ID NO: 390
  Synthetic DNA

SEQ ID NO: 449 - DNA, 59 bp, Artificial Sequence
  Synthetic DNA
atcggcaaagcggtagttgaattcggtgacgctacggccccctaacagctggcaaaatt

SEQ ID NO: 450 - DNA, 60 bp, Artificial Sequence; identical to SEQ ID NO: 392
  Synthetic DNA

SEQ ID NO: 451 - DNA, 55 bp, Artificial Sequence
  Synthetic DNA
gctcttccaggccacgggacagacggacgcccacgaaaatgcacgcatttttcgc

SEQ ID NO: 452 - DNA, 59 bp, Artificial Sequence; identical to SEQ ID NO: 394
  Synthetic DNA

SEQ ID NO: 453 - DNA, 60 bp, Artificial Sequence
  Synthetic DNA
cgtcgctgagatccaccacggaataaccaccatcgttcagcatctgcagaatctccttgc

SEQ ID NO: 454 - DNA, 55 bp, Artificial Sequence; identical to SEQ ID NO: 396
  Synthetic DNA

SEQ ID NO: 455 - DNA, 54 bp, Artificial Sequence
  Synthetic DNA
ggtgactcggacgaccccccaccatatagcgcacatgcagcttggccatttcat

SEQ ID NO: 456 - DNA, 55 bp, Artificial Sequence; identical to SEQ ID NO: 398
  Synthetic DNA

SEQ ID NO: 457 - DNA, 55 bp, Artificial Sequence
  Synthetic DNA
aacagtgcaccaggagactccggaaactcgaagctgtacaagcgttcctgcaacg

SEQ ID NO: 458 - DNA, 54 bp, Artificial Sequence; identical to SEQ ID NO: 400
  Synthetic DNA

SEQ ID NO: 459 - DNA, 59 bp, Artificial Sequence
  Synthetic DNA
gggagcgatagtgaaacaggctgatgttccagtacgtccccagggtgttcaggaagcgt

SEQ ID NO: 460 - DNA, 59 bp, Artificial Sequence; identical to SEQ ID NO: 402
  Synthetic DNA

SEQ ID NO: 461 - DNA, 54 bp, Artificial Sequence
  Synthetic DNA
gttcgtggtcccccagttcaaacgcagccaggacccgaccgtagtcagtaccat

SEQ ID NO: 462 - DNA, 55 bp, Artificial Sequence; identical to SEQ ID NO: 404
  Synthetic DNA

SEQ ID NO: 463 - DNA, 59 bp, Artificial Sequence
  Synthetic DNA
gttgttggtttcgtcatggcagtcgtagcctaattcgttcaggcgggtttcgaagtccg

SEQ ID NO: 464 - DNA, 58 bp, Artificial Sequence; identical to SEQ ID NO: 406
  Synthetic DNA

SEQ ID NO: 465 - DNA, 27 bp, Artificial Sequence
  Synthetic DNA
ccctgccagaaaaaagcgaaacgccgg

SEQ ID NO: 466 - DNA, 42 bp, Artificial Sequence
  Synthetic DNA
gcggcagccatattaccctgccagaaaaaagcgaaacgccgg

SEQ ID NO: 467 - DNA, 15 bp, Artificial Sequence
  Synthetic DNA
taatatggctgccgc

SEQ ID NO: 468 - DNA, 57 bp, Artificial Sequence
  Synthetic DNA
ttaagcgtaatccggaacatcgtatgggtaccctgccagaaaaaagcgaaacgccgg

SEQ ID NO: 469 - DNA, 30 bp, Artificial Sequence
  Synthetic DNA
tacccatacgatgttccggattacgcttaa

SEQ ID NO: 470 - DNA, 31 bp, Artificial Sequence
  Synthetic DNA
ctatatctagcatatgtcattcatggaccag

SEQ ID NO: 471 - DNA, 36 bp, Artificial Sequence
  Synthetic DNA
atgtcattcatggaccagattccgggcgggggtaac

SEQ ID NO: 472 - DNA, 47 bp, Artificial Sequence
  Synthetic DNA
gcaaacattctactggcaatttaggatagttacccccgcccggaatc

SEQ ID NO: 473 - DNA, 46 bp, Artificial Sequence
  Synthetic DNA
gccagtagaatgtttgccgaattttcccattcaaccaagtctgacc

SEQ ID NO: 474 - DNA, 47 bp, Artificial Sequence
  Synthetic DNA
gtttgtggctatcgtttctcccgcgaaaggtcagacttggttgaatg

SEQ ID NO: 475 - DNA, 50 bp, Artificial Sequence
  Synthetic DNA
gaaacgatagccacaaactgaagaatttcattagcgagattatgctcaac

SEQ ID NO: 476 - DNA, 50 bp, Artificial Sequence
  Synthetic DNA
catcgttaggccaagagatcatcgacatgttgagcataatctcgctaatg

SEQ ID NO: 477 - DNA, 50 bp, Artificial Sequence
  Synthetic DNA
ctcttggcctaacgatgcgtctagaattgtgtactgccgtcgtcatttac

SEQ ID NO: 478 - DNA, 50 bp, Artificial Sequence
  Synthetic DNA
caaagtcattagcccactgagcagctggattaagtaaatgacgacggcag

SEQ ID NO: 479 - DNA, 50 bp, Artificial Sequence
  Synthetic DNA
gtgggctaatgactttgtgcaagaacagggtattctcgagattacgttcg

SEQ ID NO: 480 - DNA, 48 bp, Artificial Sequence
  Synthetic DNA
gttgatacagcccctggataaatgtatcgaacgtaatctcgagaatac

SEQ ID NO: 481 - DNA, 48 bp, Artificial Sequence
  Synthetic DNA
gtattctcgagattacgttcgatacatttatccaggggctgtatcaac

SEQ ID NO: 482 - DNA, 49 bp, Artificial Sequence
  Synthetic DNA
gattttattgatatcaggcggtttataaaagtgttgatacagcccctgg

SEQ ID NO: 483 - DNA, 50 bp, Artificial Sequence
  Synthetic DNA
ccgcctgatatcaataaaatctttaacgccatcacgcagctgtccgaggc

SEQ ID NO: 484 - DNA, 49 bp, Artificial Sequence
  Synthetic DNA
ccgttgattcagacgttcaatgcctaattttgcctcggacagctgcgtg

SEQ ID NO: 485 - DNA, 49 bp, Artificial Sequence
  Synthetic DNA
gaacgtctgaatcaacggtttcggaaaatttgggatcgcatgccaccag

SEQ ID NO: 486 - DNA, 46 bp, Artificial Sequence
  Synthetic DNA
cataattgcggccttttctgtcatgaaatctggtggcatgcgatcc

SEQ ID NO: 487 - DNA, 48 bp, Artificial Sequence
  Synthetic DNA
gaaaaggccgcaattatgacgtatacccggttactgacgaaagagacc

SEQ ID NO: 488 - DNA, 49 bp, Artificial Sequence
  Synthetic DNA
ctccggcttatgcatacgtacaatattataggtctctttcgtcagtaac

SEQ ID NO: 489 - DNA, 50 bp, Artificial Sequence
  Synthetic DNA
gtatgcataagccggagaccctgaaagatgcgatggaggaagcctaccag

SEQ ID NO: 490 - DNA, 50 bp, Artificial Sequence
  Synthetic DNA
cagggaagaatcgttcggtaagggcagtggtctggtaggcttcctccatc

SEQ ID NO: 491 - DNA, 50 bp, Artificial Sequence
  Synthetic DNA
gatggaggaagcctaccagaccactgcccttaccgaacgattcttccctg

SEQ ID NO: 492 - DNA, 49 bp, Artificial Sequence
  Synthetic DNA
gatggtatctccgtccgcgtccagttcaaagccagggaagaatcgttcg

SEQ ID NO: 493 - DNA, 50 bp, Artificial Sequence
  Synthetic DNA
cggacggagataccatcataggcgcaaccactcacttgcaggaagagtac

SEQ ID NO: 494 - DNA, 50 bp, Artificial Sequence
  Synthetic DNA
caggttatcttccgaatcgtaatcagaatcgtactcttcctgcaagtgag

SEQ ID NO: 495 - DNA, 49 bp, Artificial Sequence
  Synthetic DNA
cgattcggaagataacctgacccaaaatggctacgttcacactgttagg

SEQ ID NO: 496 - DNA, 49 bp, Artificial Sequence
  Synthetic DNA
gctcatgggcttattgtatgaacgacgggtcctaacagtgtgaacgtag

SEQ ID NO: 497 - DNA, 50 bp, Artificial Sequence
  Synthetic DNA
catacaataagcccatgagcaaccatcggaaccgcagaaacaacaacccg

SEQ ID NO: 498 - DNA, 50 bp, Artificial Sequence
  Synthetic DNA
gcacagacggtttttgatgcattcttctcggctcgggttgttgtttctgc

SEQ ID NO: 499 - DNA, 49 bp, Artificial Sequence
  Synthetic DNA
catcaaaaaccgtctgtgcttttattgtaagaaagaaggccatcgactg

SEQ ID NO: 500 - DNA, 48 bp, Artificial Sequence
  Synthetic DNA
gagcttgctttacgggcacggcattcattcagtcgatggccttctttc

SEQ ID NO: 501 - DNA, 27 bp, Artificial Sequence
  Synthetic DNA
gcccgtaaagcaagctctaaccgtagc

SEQ ID NO: 502 - DNA, 17 bp, Artificial Sequence
  Synthetic DNA
gctacggttagagcttg

SEQ ID NO: 503 - DNA, 34 bp, Artificial Sequence
  Synthetic DNA
gtattggatccttattagctacggttagagcttg

SEQ ID NO: 504 - DNA, 1641 bp, Artificial Sequence
  Synthetic DNA
ctatatctagcatatgaccatcaccccggaaacctctcgcccgatcgacaccgaatcttggaaatcttactacaaatccgacccgctgtgctctgctgttctgatccacatgaaagaactgacccagcacaacgttaccccagaagacatgtccgctttccgctcctatcagaaaaagctggaactgtctgagaccttccgtaaaaactactccctggaggacgaaatgatctactaccaagatcgcctggttgtaccgattaaacaacaaaatgctgtcatgcgtctgtatcacgatcacactctgtttggtggtcactttggcgtaaccgttaccctggcgaaaatctctccgatctactattggccgaaactgcagcactctatcatccagtacatccgtacctgcgttcagtgccagctgatcaaatctcaccgcccacgtctgcatggtctcctgcaaccgctcccgatcgctgaaggtcgttggctggacatctctatggacttcgttactggtttgccgccgacctctaacaacctgaacatgatcctggtggtggtggaccgcttctctaaacgtgctcacttcatcgctacccgaaaaaccctggacgcgactcagctgatcgacctgctcttccgttacatcttctcttaccatggcttcccgcgtaccatcacctctgaccgtgacgttcgtatgactgcggacaaataccaagaactgaccaaacgtctgggtatcaaatctaccatgtcttccgctaaccacccgcagactgatggtcaatccgagcgtaccattcagaccctgaaccgtctcctgcgtgcgtatgcgtctaccaacatccagaactggcacgtttaccttccgcaaattgaattcgtttacaactccactccgactcgtactctgggtaaatctccgttcgaaatcgacctgggttacctgccaaacactccggcgatcaaatctgacgatgaagttaacgctcgttccttcaccgctgttgaactggctaaacacctgaaggcgctgaccatccagaccaaagaacagctggaacacgcgcagatcgaaatggaaaccaacaacaaccagcgtcgcaaaccactgctgttgaatattggtgatcatgttctggtacaccgtgatgcctacttcaaaaaaggtgcgtacatgaaagttcagcagatctacgttggtccattccgtgtcgttaagaaaatcaatgacaacgcgtatgaactggacctgaactcgcataagaagaagcaccgtgtgatcaacgttcagtttctgaaaaaatttgtttaccgtccggatgcgtacccgaaaaacaaaccgatctcttctaccgaacgcatcaaacgagctcacgaagttaccgcgctgatcggcatcgacaccacccacaaaacctatctgtgccacatgcaggacgttgacccgaccctgtccgttgaatactccgaagctgaattctgccagatcccagagcgtacccgtcgttctatcctggcgaacttccgtcagctgtacgaaacccaagacaaccctgaacgtgaagaagatgttgtttcccagaacgaaatctgccagtacgacaacacctctccgtaataaggatccaatac

SEQ ID NO: 505 - DNA, 46 bp, Artificial Sequence
  Synthetic DNA
ctatatctagcatatgaccatcaccccggaaacctctcgcccgatc

SEQ ID NO: 506 - DNA, 25 bp, Artificial Sequence
  Synthetic DNA
accatcaccccggaaacctctcgcc

SEQ ID NO: 507 - DNA, 50 bp, Artificial Sequence
  Synthetic DNA
accatcaccccggaaacctctcgcccgatcgacaccgaatcttggaaatc

SEQ ID NO: 508 - DNA, 50 bp, Artificial Sequence
  Synthetic DNA
cgatcgacaccgaatcttggaaatcttactacaaatccgacccgctgtgc

SEQ ID NO: 509 - DNA, 50 bp, Artificial Sequence
  Synthetic DNA
ttactacaaatccgacccgctgtgctctgctgttctgatccacatgaaag

SEQ ID NO: 510 - DNA, 49 bp, Artificial Sequence
  Synthetic DNA
tctgctgttctgatccacatgaaagaactgacccagcacaacgttaccc

SEQ ID NO: 511 - DNA, 45 bp, Artificial Sequence
  Synthetic DNA
aactgacccagcacaacgttaccccagaagacatgtccgctttcc

SEQ ID NO: 512 - DNA, 46 bp, Artificial Sequence
  Synthetic DNA
cagaagacatgtccgctttccgctcctatcagaaaaagctggaact

SEQ ID NO: 513 - DNA, 48 bp, Artificial Sequence
  Synthetic DNA
gctcctatcagaaaaagctggaactgtctgagaccttccgtaaaaact

SEQ ID NO: 514 - DNA, 48 bp, Artificial Sequence; identical to SEQ ID NO: 513
  Synthetic DNA

SEQ ID NO: 515 - DNA, 50 bp, Artificial Sequence
  Synthetic DNA
gtctgagaccttccgtaaaaactactccctggaggacgaaatgatctact

SEQ ID NO: 516 - DNA, 50 bp, Artificial Sequence
  Synthetic DNA
actccctggaggacgaaatgatctactaccaagatcgcctggttgtaccg

SEQ ID NO: 517 - DNA, 49 bp, Artificial Sequence
  Synthetic DNA
accaagatcgcctggttgtaccgattaaacaacaaaatgctgtcatgcg

SEQ ID NO: 518 - DNA, 50 bp, Artificial Sequence
  Synthetic DNA
attaaacaacaaaatgctgtcatgcgtctgtatcacgatcacactctgtt

SEQ ID NO: 519 - DNA, 50 bp, Artificial Sequence
  Synthetic DNA
tctgtatcacgatcacactctgtttggtggtcactttggcgtaaccgtta

SEQ ID NO: 520 - DNA, 50 bp, Artificial Sequence
  Synthetic DNA
tggtggtcactttggcgtaaccgttaccctggcgaaaatctctccgatct

SEQ ID NO: 521 - DNA, 50 bp, Artificial Sequence
  Synthetic DNA
ccctggcgaaaatctctccgatctactattggccgaaactgcagcactct

SEQ ID NO: 522 - DNA, 50 bp, Artificial Sequence; identical to SEQ ID NO: 521
  Synthetic DNA

SEQ ID NO: 523 - DNA, 50 bp, Artificial Sequence
  Synthetic DNA
actattggccgaaactgcagcactctatcatccagtacatccgtacctgc

SEQ ID NO: 524 - DNA, 50 bp, Artificial Sequence
  Synthetic DNA
atcatccagtacatccgtacctgcgttcagtgccagctgatcaaatctca

SEQ ID NO: 525 - DNA, 50 bp, Artificial Sequence
  Synthetic DNA
gttcagtgccagctgatcaaatctcaccgcccacgtctgcatggtctcct

SEQ ID NO: 526 - DNA, 50 bp, Artificial Sequence
  Synthetic DNA
ccgcccacgtctgcatggtctcctgcaaccgctcccgatcgctgaaggtc

SEQ ID NO: 527 - DNA, 50 bp, Artificial Sequence
  Synthetic DNA
gcaaccgctcccgatcgctgaaggtcgttggctggacatctctatggact

SEQ ID NO: 528 - DNA, 47 bp, Artificial Sequence
  Synthetic DNA
gttggctggacatctctatggacttcgttactggtttgccgccgacc

SEQ ID NO: 529 - DNA, 50 bp, Artificial Sequence
  Synthetic DNA
tcgttactggtttgccgccgacctctaacaacctgaacatgatcctggtg

SEQ ID NO: 530 - DNA, 50 bp, Artificial Sequence; identical to SEQ ID NO: 529
  Synthetic DNA

SEQ ID NO: 531 - DNA, 50 bp, Artificial Sequence
  Synthetic DNA
tctaacaacctgaacatgatcctggtggtggtggaccgcttctctaaacg

SEQ ID NO: 532 - DNA, 50 bp, Artificial Sequence
  Synthetic DNA
gtggtggaccgcttctctaaacgtgctcacttcatcgctacccgaaaaac

SEQ ID NO: 533 - DNA, 50 bp, Artificial Sequence
  Synthetic DNA
tgctcacttcatcgctacccgaaaaaccctggacgcgactcagctgatcg

SEQ ID NO: 534 - DNA, 50 bp, Artificial Sequence
  Synthetic DNA
cctggacgcgactcagctgatcgacctgctcttccgttacatcttctctt

SEQ ID NO: 535 - DNA, 50 bp, Artificial Sequence
  Synthetic DNA
acctgctcttccgttacatcttctcttaccatggcttcccgcgtaccatc

SEQ ID NO: 536 - DNA, 50 bp, Artificial Sequence
  Synthetic DNA
accatggcttcccgcgtaccatcacctctgaccgtgacgttcgtatgact

SEQ ID NO: 537 - DNA, 50 bp, Artificial Sequence
  Synthetic DNA
acctctgaccgtgacgttcgtatgactgcggacaaataccaagaactgac

SEQ ID NO: 538 - DNA, 50 bp, Artificial Sequence; identical to SEQ ID NO: 537
  Synthetic DNA

SEQ ID NO: 539 - DNA, 50 bp, Artificial Sequence
  Synthetic DNA
gcggacaaataccaagaactgaccaaacgtctgggtatcaaatctaccat

SEQ ID NO: 540 - DNA, 50 bp, Artificial Sequence
  Synthetic DNA
caaacgtctgggtatcaaatctaccatgtcttccgctaaccacccgcaga

SEQ ID NO: 541 - DNA, 50 bp, Artificial Sequence
  Synthetic DNA
gtcttccgctaaccacccgcagactgatggtcaatccgagcgtaccattc

SEQ ID NO: 542 - DNA, 50 bp, Artificial Sequence
  Synthetic DNA
ctgatggtcaatccgagcgtaccattcagaccctgaaccgtctcctgcgt

SEQ ID NO: 543 - DNA, 50 bp, Artificial Sequence
  Synthetic DNA
agaccctgaaccgtctcctgcgtgcgtatgcgtctaccaacatccagaac

SEQ ID NO: 544 - DNA, 50 bp, Artificial Sequence
  Synthetic DNA
gcgtatgcgtctaccaacatccagaactggcacgtttaccttccgcaaat

SEQ ID NO: 545 - DNA, 50 bp, Artificial Sequence
  Synthetic DNA
tggcacgtttaccttccgcaaattgaattcgtttacaactccactccgac

SEQ ID NO: 546 - DNA, 50 bp, Artificial Sequence; identical to SEQ ID NO: 545
  Synthetic DNA

SEQ ID NO: 547 - DNA, 50 bp, Artificial Sequence
  Synthetic DNA
tgaattcgtttacaactccactccgactcgtactctgggtaaatctccgt

SEQ ID NO: 548 - DNA, 50 bp, Artificial Sequence
  Synthetic DNA
tcgtactctgggtaaatctccgttcgaaatcgacctgggttacctgccaa

SEQ ID NO: 549 - DNA, 50 bp, Artificial Sequence
  Synthetic DNA
tcgaaatcgacctgggttacctgccaaacactccggcgatcaaatctgac

SEQ ID NO: 550 - DNA, 50 bp, Artificial Sequence
  Synthetic DNA
acactccggcgatcaaatctgacgatgaagttaacgctcgttccttcacc

SEQ ID NO: 551 - DNA, 50 bp, Artificial Sequence
  Synthetic DNA
gatgaagttaacgctcgttccttcaccgctgttgaactggctaaacacct

SEQ ID NO: 552 - DNA, 50 bp, Artificial Sequence
  Synthetic DNA
gctgttgaactggctaaacacctgaaggcgctgaccatccagaccaaaga

SEQ ID NO: 553 - DNA, 50 bp, Artificial Sequence
  Synthetic DNA
gaaggcgctgaccatccagaccaaagaacagctggaacacgcgcagatcg

SEQ ID NO: 554 - DNA, 50 bp, Artificial Sequence; identical to SEQ ID NO: 553
  Synthetic DNA

SEQ ID NO: 555 - DNA, 50 bp, Artificial Sequence
  Synthetic DNA
acagctggaacacgcgcagatcgaaatggaaaccaacaacaaccagcgtc

SEQ ID NO: 556 - DNA, 50 bp, Artificial Sequence
  Synthetic DNA
aaatggaaaccaacaacaaccagcgtcgcaaaccactgctgttgaatatt

SEQ ID NO: 557 - DNA, 50 bp, Artificial Sequence
  Synthetic DNA
gcaaaccactgctgttgaatattggtgatcatgttctggtacaccgtgat

SEQ ID NO: 558 - DNA, 50 bp, Artificial Sequence
  Synthetic DNA
ggtgatcatgttctggtacaccgtgatgcctacttcaaaaaaggtgcgta

SEQ ID NO: 559 - DNA, 50 bp, Artificial Sequence
  Synthetic DNA
gcctacttcaaaaaaggtgcgtacatgaaagttcagcagatctacgttgg

SEQ ID NO: 560 - DNA, 50 bp, Artificial Sequence
  Synthetic DNA
catgaaagttcagcagatctacgttggtccattccgtgtcgttaagaaaa

SEQ ID NO: 561 - DNA, 47 bp, Artificial Sequence
  Synthetic DNA
tccattccgtgtcgttaagaaaatcaatgacaacgcgtatgaactgg

SEQ ID NO: 562 - DNA, 47 bp, Artificial Sequence; identical to SEQ ID NO: 561
  Synthetic DNA

SEQ ID NO: 563 - DNA, 50 bp, Artificial Sequence
  Synthetic DNA
tcaatgacaacgcgtatgaactggacctgaactcgcataagaagaagcac

SEQ ID NO: 564 - DNA, 50 bp, Artificial Sequence
  Synthetic DNA
acctgaactcgcataagaagaagcaccgtgtgatcaacgttcagtttctg

SEQ ID NO: 565 - DNA, 49 bp, Artificial Sequence
  Synthetic DNA
cgtgtgatcaacgttcagtttctgaaaaaatttgtttaccgtccggatg

SEQ ID NO: 566 - DNA, 50 bp, Artificial Sequence
  Synthetic DNA
aaaaaatttgtttaccgtccggatgcgtacccgaaaaacaaaccgatctc

SEQ ID NO: 567 - DNA, 50 bp, Artificial Sequence
  Synthetic DNA
cgtacccgaaaaacaaaccgatctcttctaccgaacgcatcaaacgagct

SEQ ID NO: 568 - DNA, 50 bp, Artificial Sequence; identical to SEQ ID NO: 567
  Synthetic DNA

SEQ ID NO: 569 - DNA, 50 bp, Artificial Sequence
  Synthetic DNA
ttctaccgaacgcatcaaacgagctcacgaagttaccgcgctgatcggca

SEQ ID NO: 570 - DNA, 50 bp, Artificial Sequence
  Synthetic DNA
cacgaagttaccgcgctgatcggcatcgacaccacccacaaaacctatct

SEQ ID NO: 571 - DNA, 50 bp, Artificial Sequence
  Synthetic DNA
tcgacaccacccacaaaacctatctgtgccacatgcaggacgttgacccg

SEQ ID NO: 572 - DNA, 50 bp, Artificial Sequence
  Synthetic DNA
gtgccacatgcaggacgttgacccgaccctgtccgttgaatactccgaag

SEQ ID NO: 573 - DNA, 50 bp, Artificial Sequence
  Synthetic DNA
accctgtccgttgaatactccgaagctgaattctgccagatcccagagcg

SEQ ID NO: 574 - DNA, 50 bp, Artificial Sequence; identical to SEQ ID NO: 573
  Synthetic DNA

SEQ ID NO: 575 - DNA, 50 bp, Artificial Sequence
  Synthetic DNA
ctgaattctgccagatcccagagcgtacccgtcgttctatcctggcgaac

SEQ ID NO: 576 - DNA, 50 bp, Artificial Sequence
  Synthetic DNA
tacccgtcgttctatcctggcgaacttccgtcagctgtacgaaacccaag

SEQ ID NO: 577 - DNA, 47 bp, Artificial Sequence
  Synthetic DNA
ttccgtcagctgtacgaaacccaagacaaccctgaacgtgaagaaga

SEQ ID NO: 578 - DNA, 46 bp, Artificial Sequence
  Synthetic DNA
acaaccctgaacgtgaagaagatgttgtttcccagaacgaaatctg

SEQ ID NO: 579 - DNA, 46 bp, Artificial Sequence
  Synthetic DNA
tgttgtttcccagaacgaaatctgccagtacgacaacacctctccg

SEQ ID NO: 580 - DNA, 22 bp, Artificial Sequence
  Synthetic DNA
ccagtacgacaacacctctccg

SEQ ID NO: 581 - DNA, 47 bp, Artificial Sequence
  Synthetic DNA
gaaatctgccagtacgacaacacctctccgtaataaggatccaatac

SEQ ID NO: 582 - DNA, 46 bp, Artificial Sequence; identical to SEQ ID NO: 505
  Synthetic DNA

SEQ ID NO: 583 - DNA, 25 bp, Artificial Sequence
  Synthetic DNA
accatcaccccggaaacctctcgcc

SEQ ID NO: 584 - DNA, 50 bp, Artificial Sequence
  Synthetic DNA
gatttccaagattcggtgtcgatcgggcgagaggtttccggggtgatggt

SEQ ID NO: 585 - DNA, 50 bp, Artificial Sequence; identical to SEQ ID NO: 508
  Synthetic DNA

SEQ ID NO: 586 - DNA, 50 bp, Artificial Sequence
  Synthetic DNA
ctttcatgtggatcagaacagcagagcacagcgggtcggatttgtagtaa

SEQ ID NO: 587 - DNA, 49 bp, Artificial Sequence; identical to SEQ ID NO: 510
  Synthetic DNA

SEQ ID NO: 588 - DNA, 45 bp, Artificial Sequence
  Synthetic DNA
ggaaagcggacatgtcttctggggtaacgttgtgctgggtcagtt

SEQ ID NO: 589 - DNA, 46 bp, Artificial Sequence; identical to SEQ ID NO: 512
  Synthetic DNA

SEQ ID NO: 590 - DNA, 48 bp, Artificial Sequence
  Synthetic DNA
agtttttacggaaggtctcagacagttccagctttttctgataggagc

SEQ ID NO: 591 - DNA, 48 bp, Artificial Sequence; identical to SEQ ID NO: 513
  Synthetic DNA

SEQ ID NO: 592 - DNA, 50 bp, Artificial Sequence
  Synthetic DNA
agtagatcatttcgtcctccagggagtagtttttacggaaggtctcagac

SEQ ID NO: 593 - DNA, 50 bp, Artificial Sequence; identical to SEQ ID NO: 516
  Synthetic DNA

SEQ ID NO: 594 - DNA, 49 bp, Artificial Sequence
  Synthetic DNA
cgcatgacagcattttgttgtttaatcggtacaaccaggcgatcttggt

SEQ ID NO: 595 - DNA, 50 bp, Artificial Sequence; identical to SEQ ID NO: 518
  Synthetic DNA

SEQ ID NO: 596 - DNA, 50 bp, Artificial Sequence
  Synthetic DNA
taacggttacgccaaagtgaccaccaaacagagtgtgatcgtgatacaga

SEQ ID NO: 597 - DNA, 50 bp, Artificial Sequence; identical to SEQ ID NO: 520
  Synthetic DNA

SEQ ID NO: 598 - DNA, 50 bp, Artificial Sequence
  Synthetic DNA
agagtgctgcagtttcggccaatagtagatcggagagattttcgccaggg

SEQ ID NO: 599 - DNA, 50 bp, Artificial Sequence; identical to SEQ ID NO: 521
  Synthetic DNA

SEQ ID NO: 600 - DNA, 50 bp, Artificial Sequence
  Synthetic DNA
gcaggtacggatgtactggatgatagagtgctgcagtttcggccaatagt

SEQ ID NO: 601 - DNA, 50 bp, Artificial Sequence; identical to SEQ ID NO: 524
  Synthetic DNA

SEQ ID NO: 602 - DNA, 50 bp, Artificial Sequence
  Synthetic DNA
aggagaccatgcagacgtgggcggtgagatttgatcagctggcactgaac

SEQ ID NO: 603 - DNA, 50 bp, Artificial Sequence; identical to SEQ ID NO: 526
  Synthetic DNA

SEQ ID NO: 604 - DNA, 50 bp, Artificial Sequence
  Synthetic DNA
agtccatagagatgtccagccaacgaccttcagcgatcgggagcggttgc

SEQ ID NO: 605 - DNA, 47 bp, Artificial Sequence; identical to SEQ ID NO: 528
  Synthetic DNA

SEQ ID NO: 606 - DNA, 50 bp, Artificial Sequence
  Synthetic DNA
caccaggatcatgttcaggttgttagaggtcggcggcaaaccagtaacga

SEQ ID NO: 607 - DNA, 50 bp, Artificial Sequence; identical to SEQ ID NO: 529
  Synthetic DNA

SEQ ID NO: 608 - DNA, 50 bp, Artificial Sequence
  Synthetic DNA
cgtttagagaagcggtccaccaccaccaggatcatgttcaggttgttaga

SEQ ID NO: 609 - DNA, 50 bp, Artificial Sequence; identical to SEQ ID NO: 532
  Synthetic DNA

SEQ ID NO: 610 - DNA, 50 bp, Artificial Sequence
  Synthetic DNA
cgatcagctgagtcgcgtccagggtttttcgggtagcgatgaagtgagca

SEQ ID NO: 611 - DNA, 50 bp, Artificial Sequence; identical to SEQ ID NO: 534
  Synthetic DNA

SEQ ID NO: 612 - DNA, 50 bp, Artificial Sequence
  Synthetic DNA
gatggtacgcgggaagccatggtaagagaagatgtaacggaagagcaggt

SEQ ID NO: 613 - DNA, 50 bp, Artificial Sequence; identical to SEQ ID NO: 536
  Synthetic DNA

SEQ ID NO: 614 - DNA, 50 bp, Artificial Sequence
  Synthetic DNA
gtcagttcttggtatttgtccgcagtcatacgaacgtcacggtcagaggt

SEQ ID NO: 615 - DNA, 50 bp, Artificial Sequence; identical to SEQ ID NO: 537
  Synthetic DNA

SEQ ID NO: 616 - DNA, 50 bp, Artificial Sequence
  Synthetic DNA
atggtagatttgatacccagacgtttggtcagttcttggtatttgtccgc

SEQ ID NO: 617 - DNA, 50 bp, Artificial Sequence; identical to SEQ ID NO: 540
  Synthetic DNA

SEQ ID NO: 618 - DNA, 50 bp, Artificial Sequence
  Synthetic DNA
gaatggtacgctcggattgaccatcagtctgcgggtggttagcggaagac

SEQ ID NO: 619 - DNA, 50 bp, Artificial Sequence; identical to SEQ ID NO: 542
  Synthetic DNA

SEQ ID NO: 620 - DNA, 50 bp, Artificial Sequence
  Synthetic DNA
gttctggatgttggtagacgcatacgcacgcaggagacggttcagggtct

SEQ ID NO: 621 - DNA, 50 bp, Artificial Sequence; identical to SEQ ID NO: 544
  Synthetic DNA

SEQ ID NO: 622 - DNA, 50 bp, Artificial Sequence
  Synthetic DNA
gtcggagtggagttgtaaacgaattcaatttgcggaaggtaaacgtgcca

SEQ ID NO: 623 - DNA, 50 bp, Artificial Sequence; identical to SEQ ID NO: 545
  Synthetic DNA

SEQ ID NO: 624 - DNA, 50 bp, Artificial Sequence
  Synthetic DNA
acggagatttacccagagtacgagtcggagtggagttgtaaacgaattca

SEQ ID NO: 625 - DNA, 50 bp, Artificial Sequence; identical to SEQ ID NO: 548
  Synthetic DNA

SEQ ID NO: 626 - DNA, 50 bp, Artificial Sequence
  Synthetic DNA
gtcagatttgatcgccggagtgtttggcaggtaacccaggtcgatttcga

SEQ ID NO: 627 - DNA, 50 bp, Artificial Sequence; identical to SEQ ID NO: 550
  Synthetic DNA

SEQ ID NO: 628 - DNA, 50 bp, Artificial Sequence
  Synthetic DNA
aggtgtttagccagttcaacagcggtgaaggaacgagcgttaacttcatc

SEQ ID NO: 629 - DNA, 50 bp, Artificial Sequence; identical to SEQ ID NO: 552
  Synthetic DNA

SEQ ID NO: 630 - DNA, 50 bp, Artificial Sequence
  Synthetic DNA
cgatctgcgcgtgttccagctgttctttggtctggatggtcagcgccttc

SEQ ID NO: 631 - DNA, 50 bp, Artificial Sequence; identical to SEQ ID NO: 553
  Synthetic DNA

SEQ ID NO: 632 - DNA, 50 bp, Artificial Sequence
  Synthetic DNA
gacgctggttgttgttggtttccatttcgatctgcgcgtgttccagctgt

SEQ ID NO: 633 - DNA, 50 bp, Artificial Sequence; identical to SEQ ID NO: 556
  Synthetic DNA

SEQ ID NO: 634 - DNA, 50 bp, Artificial Sequence
  Synthetic DNA
atcacggtgtaccagaacatgatcaccaatattcaacagcagtggtttgc

SEQ ID NO: 635 - DNA, 50 bp, Artificial Sequence; identical to SEQ ID NO: 558
  Synthetic DNA

SEQ ID NO: 636 - DNA, 50 bp, Artificial Sequence
  Synthetic DNA
ccaacgtagatctgctgaactttcatgtacgcaccttttttgaagtaggc

SEQ ID NO: 637 - DNA, 50 bp, Artificial Sequence; identical to SEQ ID NO: 560
  Synthetic DNA

SEQ ID NO: 638 - DNA, 47 bp, Artificial Sequence
  Synthetic DNA
ccagttcatacgcgttgtcattgattttcttaacgacacggaatgga

SEQ ID NO: 639 - DNA, 47 bp, Artificial Sequence; identical to SEQ ID NO: 561
  Synthetic DNA

SEQ ID NO: 640 - DNA, 50 bp, Artificial Sequence
  Synthetic DNA
gtgcttcttcttatgcgagttcaggtccagttcatacgcgttgtcattga

SEQ ID NO: 641 - DNA, 50 bp, Artificial Sequence; identical to SEQ ID NO: 564
  Synthetic DNA

SEQ ID NO: 642 - DNA, 49 bp, Artificial Sequence
  Synthetic DNA
catccggacggtaaacaaattttttcagaaactgaacgttgatcacacg

SEQ ID NO: 643 - DNA, 50 bp, Artificial Sequence; identical to SEQ ID NO: 566
  Synthetic DNA

SEQ ID NO: 644 - DNA, 50 bp, Artificial Sequence
  Synthetic DNA
agctcgtttgatgcgttcggtagaagagatcggtttgtttttcgggtacg

SEQ ID NO: 645 - DNA, 50 bp, Artificial Sequence; identical to SEQ ID NO: 567
  Synthetic DNA

SEQ ID NO: 646 - DNA, 50 bp, Artificial Sequence
  Synthetic DNA
tgccgatcagcgcggtaacttcgtgagctcgtttgatgcgttcggtagaa

SEQ ID NO: 647 - DNA, 50 bp, Artificial Sequence; identical to SEQ ID NO: 570
  Synthetic DNA

SEQ ID NO: 648 - DNA, 50 bp, Artificial Sequence
  Synthetic DNA
cgggtcaacgtcctgcatgtggcacagataggttttgtgggtggtgtcga

SEQ ID NO: 649 - DNA, 50 bp, Artificial Sequence; identical to SEQ ID NO: 572
  Synthetic DNA

SEQ ID NO: 650 - DNA, 50 bp, Artificial Sequence
  Synthetic DNA
cgctctgggatctggcagaattcagcttcggagtattcaacggacagggt

SEQ ID NO: 651 - DNA, 50 bp, Artificial Sequence; identical to SEQ ID NO: 573
  Synthetic DNA

SEQ ID NO: 652 - DNA, 50 bp, Artificial Sequence
  Synthetic DNA
gttcgccaggatagaacgacgggtacgctctgggatctggcagaattcag

SEQ ID NO: 653 - DNA, 50 bp, Artificial Sequence; identical to SEQ ID NO: 576
  Synthetic DNA

SEQ ID NO: 654 - DNA, 47 bp, Artificial Sequence
  Synthetic DNA
tcttcttcacgttcagggttgtcttgggtttcgtacagctgacggaa

SEQ ID NO: 655 - DNA, 46 bp, Artificial Sequence; identical to SEQ ID NO: 578
  Synthetic DNA

SEQ ID NO: 656 - DNA, 46 bp, Artificial Sequence
  Synthetic DNA
cggagaggtgttgtcgtactggcagatttcgttctgggaaacaaca

SEQ ID NO: 657 - DNA, 22 bp, Artificial Sequence
  Synthetic DNA
ccagtacgacaacacctctccg

SEQ ID NO: 658 - DNA, 21 bp, Artificial Sequence
  Synthetic DNA
cggagaggtgttgtcgtactg

SEQ ID NO: 659 - DNA, 47 bp, Artificial Sequence
  Synthetic DNA
gtattggatccttattacggagaggtgttgtcgtactggcagatttc

SEQ ID NO: 660 - DNA, 2743 bp, Artificial Sequence
  Synthetic DNA
gggcgaattcgagctcggtacccggggatcctctagagtcgacctgcaggcatgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 661 - DNA, 2739 bp, Artificial Sequence
  Synthetic DNA
gggcgcgagctcggtacccggggatcctctagagtcgacctgcaggcatgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 662 - DNA, 2739 bp, Artificial Sequence
  Synthetic DNA
tatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagcttgcatgcctgcaggtcgactctagaggatccccgggtaccgagctcgcgccc

SEQ ID NO: 663 - DNA, 178 bp, Artificial Sequence
  Synthetic DNA
gatcctccattccgtgtcgttaagaaaatcaatgacaacgcgtatgaactggacctgaactcgcataagaagaagcaccgtgtgatcaacgttcagtttctgaaaaaatttgtttaccgtccggatgcgtacccgaaaaacaaaccgatctcttctaccgaacgcatcaaacgagctg

SEQ ID NO: 664 - DNA, 2856 bp, Artificial Sequence
  Synthetic DNA
atggaaaagacatataacccacaagatatcgaacagccgctttacgagcactgggaaaagcagggctactttaagcctaatggcgatgaaagccaggaaagtttctgcatcatgatcccgccgccgaacgtcaccggcagtttgcatatgggtcacgccttccagcaaaccatcatggataccatgatccgctatcagcgcatgcagggcaaaaacaccctgtggcaggtcggtactgaccacgccgggatcgctacccagatggtcgttgagcgcaagattgccgcagaagaaggtaaaacccgtcacgactacggccgcgaagctttcatcgacaaaatctgggaatggaaagcggaatctggcggcaccattacccgtcagatgcgccgtctcggcaactccgtcgactgggagcgtgaacgcttcaccatggacgaaggcctgtccaatgcggtgaaagaagttttcgttcgtctgtataaagaagacctgatttaccgtggcaaacgcctggtaaactgggatccgaaactgcgcaccgctatctctgacctggaagtcgaaaaccgcgaatcgaaaggttcgatgtggcacatccgctatccgctggctgacggtgcgaaaaccgcagacggtaaagattatctggtggtcgcgactacccgtccagaaaccctgctgggcgatactggcgtagccgttaacccggaagatccgcgttacaaagatctgattggcaaatatgtcattctgccgctggttaaccgtcgtattccgatcgttggcgacgaacacgccgacatggaaaaaggcaccggctgcgtgaaaatcactccggcgcacgactttaacgactatgaagtgggtaaacgtcacgccctgccgatgatcaacatcctgacctttgacggcgatatccgtgaaagcgcccaggtgttcgataccaaaggtaacgaatctgacgtttattccagcgaaatccctgcagagttccagaaactggagcgttttgctgcacgtaaagcagtcgttgccgcagttgacgcgcttggcctgctggaagaaattaaaccgcacgacctgaccgttccttacggcgaccgtggcggcgtagttatcgaaccaatgctgaccgaccagtggtacgtgcgtgccgatgtcctggcgaaaccggcggttgaagcggttgagaacggcgacattcagttcgtaccgaagcagtacgaaaacatgtacttctcctggatgcgcgatattcaggactggtgtatctctcgtcagttgtggtggggtcaccgtatcccggcatggtatgacgaagcgggtaacgtttatgttggccgcaacgaagacgaagtgcgtaaagaaaataacctcggtgctgatgttgtcctgcgtcaggacgaagacgttctcgatacctggttctcttctgcgctgtggaccttctctacccttggctggccggaaaataccgacgccctgcgtcagttccacccaaccagcgtgatggtatctggtttcgacatcattttcttctggattgcccgcatgatcatgatgaccatgcacttcatcaaagatgaaaatggcaaaccgcaggtgccgttccacaccgtttacatgaccggcctgattcgtgatgacgaaggccagaagatgtccaaatccaagggtaacgttatcgacccactggatatggttgacggtatttcgctgccagaactgctggaaaaacgtaccggcaatatgatgcagccgcagctggcggacaaaatccgtaagcgcaccgagaagcagttcccgaacggtattgagccgcacggtactgacgcgctgcgcttcaccctggcggcgctggcgtctaccggtcgtgacatcaactgggatatgaagcgtctggaaggttaccgtaacttctgtaacaagctgtggaacgccagccgctttgtgctgatgaacacagaaggtcaggattgcggcttcaacggcggcgaaatgacgctgtcgctggcggaccgctggattctggcggagttcaaccagaccatcaaagcgtaccgcgaagcgctggacagcttccgcttcgatatcgccgcaggcattctgtatgagttcacctggaaccagttctgtgactggtatctcgagctgaccaagccggtaatgaacggtggcaccgaagcagaactgcgcggtactcgccatacgctggtgactgtactggaaggtctgctgcgcctcgcgcatccgatcattccgttcatcaccgaaaccatctggcagcgtgtgaaagtactttgcggtatcactgccgacaccatcatgctgcagccgttcccgcagtacgatgcatctcaggttgatgaagccgcactggccgacaccgaatggctgaaacaggcgatcgttgcggtacgtaacatccgtgcagaaatgaacatcgcgccgggcaaaccgctggagctgctgctgcgtggttgcagcgcggatgcagaacgtcgcgtaaatgaaaaccgtggcttcctgcaaaccctggcgcgtctggaaagtatcaccgtgctgcctgccgatgacaaaggtccggtttccgttacgaagatcatcgacggtgcagagctgctgatcccgatggctggcctcatcaacaaagaagatgagctggcgcgtctggcgaaagaagtggcgaagattgaaggtgaaatcagccgtatcgagaacaaactggcgaacgaaggctttgtcgcccgcgcaccggaagcggtcatcgcgaaagagcgtgagaagctggaaggctatgcggaagcgaaagcgaaactgattgaacagcaggctgttatcgccgcgctgtaa

SEQ ID NO: 665 - DNA, 45 bp, Artificial Sequence
  Synthetic DNA
cggcaacctggaaataatggagctcaaagacatataacccacaag

SEQ ID NO: 666 - DNA, 45 bp, Artificial Sequence
  Synthetic DNA
cttgtgggttatatgtctttgagctccattatttccaggttgccg

SEQ ID NO: 667 - DNA, 2860 bp, Artificial Sequence
  Synthetic DNA
atggagctcaaagacatataacccacaagatatcgaacagccgctttacgagcactgggaaaagcagggctactttaagcctaatggcgatgaaagccaggaaagtttctgcatcatgatcccgccgccgaacgtcaccggcagtttgcatatgggtcacgccttccagcaaaccatcatggataccatgatccgctatcagcgcatgcagggcaaaaacaccctgtggcaggtcggtactgaccacgccgggatcgctacccagatggtcgttgagcgcaagattgccgcagaagaaggtaaaacccgtcacgactacggccgcgaagctttcatcgacaaaatctgggaatggaaagcggaatctggcggcaccattacccgtcagatgcgccgtctcggcaactccgtcgactgggagcgtgaacgcttcaccatggacgaaggcctgtccaatgcggtgaaagaagttttcgttcgtctgtataaagaagacctgatttaccgtggcaaacgcctggtaaactgggatccgaaactgcgcaccgctatctctgacctggaagtcgaaaaccgcgaatcgaaaggttcgatgtggcacatccgctatccgctggctgacggtgcgaaaaccgcagacggtaaagattatctggtggtcgcgactacccgtccagaaaccctgctgggcgatactggcgtagccgttaacccggaagatccgcgttacaaagatctgattggcaaatatgtcattctgccgctggttaaccgtcgtattccgatcgttggcgacgaacacgccgacatggaaaaaggcaccggctgcgtgaaaatcactccggcgcacgactttaacgactatgaagtgggtaaacgtcacgccctgccgatgatcaacatcctgacctttgacggcgatatccgtgaaagcgcccaggtgttcgataccaaaggtaacgaatctgacgtttattccagcgaaatccctgcagagttccagaaactggagcgttttgctgcacgtaaagcagtcgttgccgcagttgacgcgcttggcctgctggaagaaattaaaccgcacgacctgaccgttccttacggcgaccgtggcggcgtagttatcgaaccaatgctgaccgaccagtggtacgtgcgtgccgatgtcctggcgaaaccggcggttgaagcggttgagaacggcgacattcagttcgtaccgaagcagtacgaaaacatgtacttctcctggatgcgcgatattcaggactggtgtatctctcgtcagttgtggtggggtcaccgtatcccggcatggtatgacgaagcgggtaacgtttatgttggccgcaacgaagacgaagtgcgtaaagaaaataacctcggtgctgatgttgtcctgcgtcaggacgaagacgttctcgatacctggttctcttctgcgctgtggaccttctctacccttggctggccggaaaataccgacgccctgcgtcagttccacccaaccagcgtgatggtatctggtttcgacatcattttcttctggattgcccgcatgatcatgatgaccatgcacttcatcaaagatgaaaatggcaaaccgcaggtgccgttccacaccgtttacatgaccggcctgattcgtgatgacgaaggccagaagatgtccaaatccaagggtaacgttatcgacccactggatatggttgacggtatttcgctgccagaactgctggaaaaacgtaccggcaatatgatgcagccgcagctggcggacaaaatccgtaagcgcaccgagaagcagttcccgaacggtattgagccgcacggtactgacgcgctgcgcttcaccctggcggcgctggcgtctaccggtcgtgacatcaactgggatatgaagcgtctggaaggttaccgtaacttctgtaacaagctgtggaacgccagccgctttgtgctgatgaacacagaaggtcaggattgcggcttcaacggcggcgaaatgacgctgtcgctggcggaccgctggattctggcggagttcaaccagaccatcaaagcgtaccgcgaagcgctggacagcttccgcttcgatatcgccgcaggcattctgtatgagttcacctggaaccagttctgtgactggtatctcgagctgaccaagccggtaatgaacggtggcaccgaagcagaactgcgcggtactcgccatacgctggtgactgtactggaaggtctgctgcgcctcgcgcatccgatcattccgttcatcaccgaaaccatctggcagcgtgtgaaagtactttgcggtatcactgccgacaccatcatgctgcagccgttcccgcagtacgatgcatctcaggttgatgaagccgcactggccgacaccgaatggctgaaacaggcgatcgttgcggtacgtaacatccgtgcagaaatgaacatcgcgccgggcaaaccgctggagctgctgctgcgtggttgcagcgcggatgcagaacgtcgcgtaaatgaaaaccgtggcttcctgcaaaccctggcgcgtctggaaagtatcaccgtgctgcctgccgatgacaaaggtccggtttccgttacgaagatcatcgacggtgcagagctgctgatcccgatggctggcctcatcaacaaagaagatgagctggcgcgtctggcgaaagaagtggcgaagattgaaggtgaaatcagccgtatcgagaacaaactggcgaacgaaggctttgtcgcccgcgcaccggaagcggtcatcgcgaaagagcgtgagaagctggaaggctatgcggaagcgaaagcgaaactgattgaacagcaggctgttatcgccgcgctgtaa

(19) United States
(12) Patent Application Publication
(10) Pub. No.: US 2005/0106590 A1
(43) Pub. Date: May 19, 2005

(54) Method for producing a synthetic gene or other DNA sequence

(76) Inventors: Richard H. Lathrop, Irvine, CA (US); Liza S.Z. Larsen, Irvine, CA (US); Christopher Wassman, Huntington Beach, CA (US); G. Wesley Hatfield, Corona del Mar, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/903,632

(22) Filed: Jul. 30, 2004

Related U.S. Application Data
(63) Continuation-in-part of application No. 10/851,383, filed on May 21, 2004.
(60) Provisional application No. 60/472,822, filed on May 22, 2003.

(51) Int. Cl. C12Q 1/68; C12P 19/34

(52) U.S. Cl. 435/6; 435/91.2

(57) ABSTRACT

Disclosed herein is a method for synthesizing a desired nucleic acid sequence. The method comprises dividing the desired sequence into a plurality of partially overlapping segments; optimizing the melting temperatures of the overlapping regions of each segment to disfavor hybridization to the overlapping segments which are non-adjacent in the desired sequence; allowing the overlapping regions of single stranded segments which are adjacent to one another in the desired sequence to hybridize to one another under conditions which disfavor hybridization of non-adjacent segments; and filling in, ligating, or repairing the gaps between the overlapping regions, thereby forming a double-stranded DNA with the desired sequence. Also disclosed is a method for preventing errors in the synthesis of the nucleic acid sequence.

### CROSS-REFERENCE TO RELATED APPLICATION

This application is a continuation-in-part of U.S. application Ser. No. 10/851,383, filed May 21, 2004, which claims the benefit of U.S. Provisional Patent Application No. 60/472,822, filed on May 22, 2003, the disclosures of which are incorporated by reference.

### BACKGROUND OF THE INVENTION

1. Field of the Invention

The present application is generally related to the synthesis of DNA molecules, and more particularly, to the synthesis of a synthetic gene or other DNA sequence.

2. Description of the Related Art

Proteins are an important class of biological molecules that have a wide range of valuable medical, pharmaceutical, industrial, and biological applications. A gene encodes the information necessary to produce a protein according to the genetic code by using three nucleotides (one codon or set of codons) for each amino acid in the protein. An expression vector contains DNA sequences that allow transcription of the gene into mRNA for translation into a protein.

It is often desirable to obtain a synthetic DNA which encodes the protein of interest. DNA can be synthesized accurately in short pieces, say 50 to 80 nucleotides or less. Pieces substantially longer than this become problematic due to cumulative error probability in the synthesis process. Most genes are appreciably longer than 50 to 80 nucleotides, usually by hundreds or thousands of nucleotides. Consequently, direct synthesis is not a convenient method for producing large genes. Currently, large synthetic genes with a desired DNA sequence are manufactured by any one of several methods:

1. If the gene does not contain introns it can be synthesized by PCR directly from genomic DNA. This is feasible for genes of bacteria, lower eukaryotes, and many viruses, but nearly all genes of higher organisms contain introns.

2. A related alternative is to PCR the gene from a full-length cDNA clone. It is time consuming and tedious to isolate and characterize a full-length clone, and full-length cDNA clones are available for only a very small fraction of the genes of any higher organism.

3. Based on the gene sequence inferred from the genomic DNA sequence, short DNA segments of both strands of a gene can be synthesized with overlapping ends. These segments are allowed to anneal and are joined together with DNA ligase. Annealing efficiency and accuracy at the segment junctions is often poor, resulting in low yields.

4. An approach to reduce this problem is to build the gene up in subsections in a step-wise manner. This remains time-consuming, expensive, tedious, and inefficient, because many reactions must be performed.

5. Based on the gene sequence inferred from the genomic DNA sequence, short overlapping duplex DNA segments of the gene can be synthesized that contain compatible end-proximal restriction endonuclease sites. Each fragment can be cut with the appropriate enzyme, annealed, and joined with DNA ligase. In addition to the limitations above, both strands of the gene sequence must be synthesized and this method is dependent on the placement of appropriate restriction sites evenly spaced throughout the gene sequence.

6. Genes have also been assembled by overlap extension of partially overlapping oligonucleotides using DNA-polymerase-catalyzed reactions. The gene is divided into oligonucleotides, each of which partially overlaps and is complementary to the adjacent oligonucleotide(s). The oligonucleotides are allowed to anneal and the resulting DNA construct is extended to the full-length double-stranded gene. See, for example, W. P. C. Stemmer et al. “Single-step assembly of a gene and entire plasmid from large numbers of oligodeoxyribonucleotides” Gene, 1995, 164, 49-53 and D. E. Casimiro et al. “PCR-based gene synthesis and protein NMR spectroscopy” Structure, 1997, 5, 1407-1412, the disclosures of which are incorporated by reference. Designing the oligonucleotides for gene synthesis by this approach has recently been automated as described in D. M. Hoover & J. Lubkowski “DNA Works: an automated method for designing oligonucleotides for PCR-based gene synthesis” Nucleic Acids Res., 2002, 30:10 e43, the disclosure of which is incorporated by reference. The method optimizes codon usage, optionally removes DNA hairpins, and uses a nearest-neighbor model of DNA melting to achieve homogeneous target melting temperatures. The process of removing local DNA hairpins and dimerization from a single DNA oligonucleotide is also referred to by those skilled in the art as “removing DNA secondary structure.” The methods described in these references do not globally optimize a melting temperature gap between correct hybridizations and incorrect hybridizations, however.

### SUMMARY OF THE INVENTION

The present application provides a method for synthesizing a DNA sequence and the DNA sequences synthesized by the method. A preferred embodiment of the method utilizes the flexibility of the genetic code to achieve melting temperatures that optimize the simultaneous annealing of many gene segments in the desired order, facilitating the assembly of a large strand from many small ones.

In some embodiments, the likelihood of synthesizing the correct DNA sequence from a mixture of correct and incorrect gene segments is increased by determining a property of the DNA sequence or fragment thereof, or of a polypeptide or protein expressed therefrom, or of another molecule derived therefrom in order to ascertain the correctness of the DNA sequence or fragment thereof. Examples of suitable properties include the DNA sequence and the molecular weight of the polypeptide expressed therefrom.

The method in the present application is of practical utility. A number of companies currently offer synthetic gene services. Consequently, a method with improved efficiency is valuable. A partial list of such companies may be generated easily, for example, by performing an Internet search for “custom gene synthesis,” “synthetic gene services,” or related keywords.

Those skilled in the art will immediately comprehend myriad applications to which the disclosed method may be applied, including: (1) creating de novo “designer” proteins; (2) coupling to automated expression and crystallization facilities; (3) building DNA sequences predicted to express novel protein folds for structural proteomics; (4) building other DNA sequences that do not encode proteins, e.g., as RNA structural templates or DNA nanotechnology components; (5) expressing proteins from a different species in a desired expression vector according to its own codon usage preference; and (6) creating a small synthetic genome by specifying its desired protein sequences and regulatory protein binding sites.

The present application provides a recursive method for synthesizing a gene of arbitrary size, i.e., a double-stranded DNA (dsDNA) sequence that codes for a desired peptide sequence, possibly with flanking regulatory and intergenic sequences extending into other flanking genes. The disclosed method uses sequence degeneracy to achieve melting temperatures that optimize the simultaneous annealing of many gene segments in the desired order. This optimization is achieved by choosing bases and codons such that, with high probability, incorrect or wrong hybridizations melt at a low temperature and correct or right hybridizations melt at a high temperature, allowing the construction of a large synthetic gene quickly and easily.

The method comprises: (a) hierarchical assembly (b) by high-fidelity techniques such as overlap extension using proof-reading DNA polymerase, ligation, cloning, or other methods, (c) with optimization of the sequences of the component oligonucleotide pieces to facilitate preferential hybridization to the desired adjacent piece(s) and to disfavor undesired hybridizations between other pieces, for example, by exploiting the degeneracy of the genetic code or a regulatory region consensus sequence, (d) to achieve a DNA melting temperature gap between correct (high melting temperature) and incorrect (low melting temperature) hybridizations, and (e) optionally selecting pieces of DNA likely to have the correct DNA sequence for subsequent assembly steps, (f) so that, with high probability correct assemblies will form. Thus, the sequence is designed to encode its own correct self-assembly in a signal superimposed on the coding sequence by using synonymous codon substitutions in a manner friendly to the expression vector.

R. M. Horton et al. “Engineering hybrid genes without the use of restriction enzymes: gene splicing by overlap extension” Gene, 1989, 77, 61-68, the disclosure of which is incorporated by reference in its entirety, discloses overlap extension, but does not disclose optimizing overlap regions to facilitate the assembly of pieces in the desired order. In particular, Horton et al. does not disclose the use of informatics to encode the self-assembly of the gene by exploiting sequence degeneracy to achieve high melting temperatures for correct hybridizations and low melting temperatures for incorrect hybridizations.

Accordingly, the present disclosure provides a method for synthesizing a DNA sequence comprising at least the steps of: (i) dividing the DNA sequence recursively into small pieces of DNA, wherein adjacent pieces comprise overlapping regions; (ii) optimizing the sequences of the pieces of DNA resulting from each recursive division to strengthen correct hybridizations and to disrupt incorrect hybridizations; (iii) obtaining the optimized small pieces of DNA, wherein the overlapping regions of any adjacent pieces of single-stranded DNA are complementary; (iv) combining the pieces of DNA derived from the division of the next larger piece of DNA; (v) allowing the pieces of DNA to self-assemble to form a DNA construct comprising single-stranded DNA segments connected by double-stranded overlap regions; (vi) producing the next-larger piece of DNA from the DNA construct; and (vii) repeating steps (iv), (v), and (vi) in reverse order of the recursive division in step (i) to produce the DNA sequence.

The present disclosure further provides a DNA sequence, synthesized according to a method comprising at least the steps of: (i) dividing the DNA sequence recursively into small pieces of DNA, wherein adjacent pieces comprise overlapping regions; (ii) optimizing the sequences of the pieces of DNA resulting from each recursive division to strengthen correct hybridizations and to disrupt incorrect hybridizations; (iii) obtaining the optimized small pieces of DNA, wherein the overlapping regions of any adjacent pieces of single-stranded DNA are complementary; (iv) combining the pieces of DNA derived from the division of the next larger piece of DNA; (v) allowing the pieces of DNA to self-assemble to form a DNA construct comprising single-stranded DNA segments connected by double-stranded overlap regions; (vi) producing the next-larger piece of DNA from the DNA construct; and (vii) repeating steps (iv), (v), and (vi) in reverse order of the recursive division in step (i) to produce the DNA sequence.

In some embodiments, a next-larger piece of DNA produced in step (vi) comprises a mixture of DNA molecules. Some embodiments further comprise a step of selecting a DNA molecule from the mixture likely to have the correct DNA sequence and using the selected DNA molecule in the synthesis of the DNA sequence. In some embodiments, a DNA molecule is separated from the mixture by cloning. In some embodiments, the selection comprises determining a property of selected DNA molecules from the mixture, or of polypeptides expressed therefrom, and selecting a DNA molecule based on a predetermined value for the property. In some embodiments, the selection comprises sequencing a sample of DNA molecules from the mixture and selecting a DNA molecule with the correct DNA sequence. In some embodiments, the selection comprises expressing a polypeptide from each member of a sample of DNA molecules from the mixture, determining the molecular weight of the polypeptide, and selecting a DNA molecule from which a polypeptide with a predetermined molecular weight is expressed. In some embodiments, a start codon and/or stop codon is incorporated into DNA molecule from which a polypeptide is expressed. In some embodiments, the reading frame of the DNA molecule is adjusted with respect to the start codon and/or stop codon. In some embodiments, one or more stop codons is inserted into the expression vector downstream (3′) from the gene. In some embodiments, the molecular weight of the polypeptide is determined by electrophoresis.

The present disclosure further provides a method for synthesizing a DNA sequence comprising at least the steps of: (i) dividing the DNA sequence recursively into small pieces of DNA, wherein adjacent pieces comprise overlapping regions; (ii) obtaining the small pieces of DNA, wherein the overlapping regions of any adjacent pieces of single-stranded DNA are complementary; (iii) combining the pieces of DNA derived from the division of the next larger piece of DNA; (iv) allowing the pieces of DNA to self-assemble to form a DNA construct comprising single-stranded DNA segments connected by double-stranded overlap regions; (v) producing the next-larger piece of DNA from the DNA construct; (vi) selecting a next-larger piece of DNA likely to have the correct sequence; and (vii) repeating steps (iii), (iv), (v), and (vi) in reverse order of the recursive division in step (i) to produce the DNA sequence.

The present disclosure further provides a DNA sequence, synthesized according to a method comprising at least the steps of: (i) dividing the DNA sequence recursively into small pieces of DNA, wherein adjacent pieces comprise overlapping regions; (ii) obtaining the small pieces of DNA, wherein the overlapping regions of any adjacent pieces of single-stranded DNA are complementary; (iii) combining the pieces of DNA derived from the division of the next larger piece of DNA; (iv) allowing the pieces of DNA to self-assemble to form a DNA construct comprising single-stranded DNA segments connected by double-stranded overlap regions; (v) producing the next-larger piece of DNA from the DNA construct; (vi) selecting a next-larger piece of DNA likely to have the correct sequence; and (vii) repeating steps (iii), (iv), (v), and (vi) in reverse order of the recursive division in step (i) to produce the DNA sequence.

In some embodiments, the DNA sequence comprises a regulatory sequence. In other embodiments, the synthetic gene has an intergenic sequence. In other embodiments, the DNA sequence has flanking regulatory and intergenic sequences extending into other flanking genes. In other embodiments, the DNA sequence encodes a polypeptide. Preferably, the polypeptide is a portion of a full-length protein. More preferably, the polypeptide is a full-length protein. In some embodiments, the DNA sequence is a synthetic genome comprising multiple flanking encoded polypeptides, their regulatory regions, and intergenic regions.

In a preferred embodiment, the sequence of the DNA sequence is divided into small pieces of DNA in a single division. This embodiment is referred to herein as “direct self-assembly.” In another preferred embodiment, the sequence of the synthetic gene is divided into small pieces of DNA in a plurality of divisions. This embodiment is referred to herein as “recursive assembly” or “hierarchical assembly.” In one embodiment, the sequence of the synthetic gene is divided into pieces of DNA of about 1,500 bases long or shorter.

The small pieces of DNA are preferably about 60 bases long or shorter, more preferably, about 50 bases long or shorter. Preferably, the overlapping regions comprise from about 6 to about 60 base-pairs, more preferably, from about 14 to about 33 base-pairs.

In a preferred embodiment, optimization is performed by calculating a melting temperature for the pieces of DNA. Preferably, the lowest correct hybridization melting temperature is higher than the highest incorrect hybridization melting temperature. Those skilled in the art will realize that the size of the melting temperature gap is related to the annealing conditions such that a narrower gap may require more stringent annealing conditions in the reassembly step to provide the requisite level of fidelity. Consequently, the temperature gap has no minimum value. Practically, the difference between the lowest-melting correct match and the highest melting incorrect match is at least about 1° C., more preferably, at least about 4° C., more preferably, at least about 8° C., most preferably, at least about 16° C. The wider the temperature gap, the more robust the self-assembly, thereby permitting the use of less stringent annealing conditions. Those skilled in the art will appreciate that optimization may be performed using other parameters or measures related to hybridization propensity, for example, free energy, enthalpy, entropy, or other arithmetic or algebraic combinations of such parameters or measures, to achieve the same effect as melting temperature. Indeed, the melting temperature itself is one such arithmetic or algebraic combination of such parameters or measures. Consequently, in some embodiments, optimization is performed by calculating a parameter related to hybridization propensity for the pieces of DNA, for example, free energy, enthalpy, entropy, and arithmetic or algebraic combinations thereof.

In some embodiments, the pieces of DNA are optimized by permuting silent codon substitutions, for example for a portion encoding a polypeptide. In some embodiments, the pieces of DNA are optimized by taking advantage of the degeneracy in the regulatory region consensus sequence, for example for a regulatory region. In some embodiments, the pieces of DNA are optimized by adjusting boundary points between adjacent pieces of DNA. In some embodiments, the pieces of DNA are optimized by direct base assignment, for example for an intergenic region.

In a preferred embodiment, at least one of the optimized small pieces of DNA is synthetic. In another preferred embodiment, at least one of the optimized small pieces of DNA is single-stranded.

In some embodiments, a single-stranded DNA segment in the DNA construct has a length of zero bases. In some embodiments, a single stranded DNA segment has a length of from about zero bases to about 20 bases.

In a preferred embodiment, the next-larger piece of DNA is produced by cloning the DNA construct and using cellular machinery. Examples of suitable cloning methods include exonuclease III cloning, topoisomerase cloning, restriction enzyme cloning, and homologous recombination cloning. In another preferred embodiment, the next-larger piece of DNA is produced by ligating the DNA construct. In yet anther preferred embodiment, the next-larger piece of DNA is produced by extending the DNA construct by a reaction using a DNA polymerase. Preferably, the DNA polymerase is a proof-reading DNA polymerase.

In a preferred embodiment, a 3′ nucleotide in an overlapping region is G or C. Preferably, both 3′ nucleotides in an overlapping region are independently G or C. In another preferred embodiment, a 3′ nucleotide in an overlapping region is A or T.

In a preferred embodiment, a DNA polymerase primer is mixed with the pieces of DNA derived from the division of the next-larger piece of DNA. In another preferred embodiment, no DNA polymerase primer is combined with the pieces of DNA derived from the division of the next-larger piece of DNA.

In a preferred embodiment, a restriction site is designed into an overlapping region. In another preferred embodiment, the restriction site is digested with a site-specific restriction enzyme.

Some embodiments provide a method of synthesizing a DNA sequence and/or the DNA sequence synthesized therewith. The method comprises at least the steps of: (i) dividing the DNA sequence recursively into small pieces of DNA, wherein adjacent pieces comprise overlapping regions; (ii) optimizing the sequences of the pieces of DNA resulting from each recursive division to strengthen correct hybridizations and to disrupt incorrect hybridizations; (iii) obtaining the optimized small pieces of DNA, wherein the overlapping regions of any adjacent pieces of single-stranded DNA are complementary; (iv) combining the pieces of DNA derived from the division of the next-larger piece of DNA; (v) allowing the pieces of DNA to self-assemble to form a DNA construct comprising single-stranded DNA segments connected by double-stranded overlap regions; (vi) producing the next-larger piece of DNA from the DNA construct; and (vii) repeating steps (iv), (v), and (vi) in reverse order of the recursive division in step (i) to produce the DNA sequence. At least one next-larger piece of DNA comprises a mixture of DNA molecules, the mixture comprising a correct DNA sequence and a DNA sequence comprising a point deletion. The method further comprises isolating a next-larger piece of DNA from the mixture by a method comprising at least the steps of: inserting the next-larger piece of DNA into a DNA insertion site in a frameshifted vector; transforming a preselected organism with the resulting vector; selecting an organism exhibiting a predetermined phenotype; and isolating the next-larger piece of DNA from the selected organism. The frameshifted vector comprises an open reading frame comprising a gene and the DNA insertion site, the gene comprises a functional portion that encodes a functional polypeptide, the expression of which changes the phenotype of the organism, the functional portion of the gene is frameshifted such that no functional polypeptide is expressed, and the DNA insertion site is upstream of the functional portion of the gene. The next-larger piece of DNA, when inserted at the DNA insertion site, corrects the frameshift, such that the functional portion of the gene expresses a functional polypeptide.

In some embodiments, the frameshifted vector is a plasmid and the preselected organism is E. coli. In some embodiments, the change in phenotype is visually apparent.

In some embodiments, change in phenotype is color. In some embodiments, the plasmid comprises a gene for the α-complementing fragment of β-galactosidase and the preselected organism is an E. coli strain with the lacZΔM 15 genotype. In some embodiments, the transformed E. coli is grown on indicator agar comprising isopropylthio-β-D-galactoside (IPTG) and 5-bromo-4-chloro-3-indolyl-β-D-galactoside (X-Gal), and wherein the predetermined phenotype is a blue colored colony. In some embodiments, the plasmid has SEQ. ID. NO.: 661 and the preselected organism is E. coli JM109.

In other embodiments, change in phenotype is growth at a restrictive temperature. In some embodiments, the plasmid comprises a gene for valyl-tRNA synthesase^{ts} and the preselected organism is E. coli AB4141. In some embodiments, the plasmid has SEQ. ID. NO.: 667.

In some embodiments, the frameshift is a −1 frameshift. In other embodiments, the frameshift is a +1 frameshift. In some embodiments, the DNA insertion site comprises a restriction site.

In some embodiments, the next-larger piece of DNA is an intermediate fragment. In other embodiments, the next-larger piece of DNA is a full-length gene.

In some embodiments, the next-larger piece of DNA is isolated by polymerase chain reaction.

Some embodiments provide a method for isolating a piece of DNA and/or a piece of DNA isolated therewith. The method comprises at least the steps of: inserting the piece of DNA into a DNA insertion site in a frameshifted vector, transforming a preselected organism with the resulting vector, selecting an organism exhibiting a predetermined phenotype, and isolating the piece of DNA from the preselected organism. The frameshifted vector comprises an open reading frame comprising a gene and the DNA insertion site, the gene comprises a functional portion that encodes a functional polypeptide, the expression of which changes the phenotype of the organism, the functional portion of the gene is frameshifted such that no functional polypeptide is expressed, and the DNA insertion site is upstream of the functional portion of the gene. The piece of DNA, when inserted at the DNA insertion site, corrects the frameshift, such that the functional portion of the gene expresses a functional polypeptide.

In some embodiments, the frameshifted vector is a plasmid and the preselected organism is E. coli. In some embodiments, the change in phenotype is visually apparent.

In some embodiments, change in phenotype is color. In some embodiments, the plasmid comprises a gene for the α-complementing fragment of β-galactosidase and the preselected organism is an E. coli strain with the lacZΔM15 genotype. In some embodiments, the transformed E. coli is grown on indicator agar comprising isopropylthio-p-D-galactoside (IPTG) and 5-bromo-4-chloro-3-indolyl-β-D-galactoside (X-Gal), and wherein the predetermined phenotype is a blue colored colony. In some embodiments, the plasmid has SEQ. ID. NO.: 661 and the preselected organism is E. coli JM109.

In other embodiments, change in phenotype is growth at a restrictive temperature. In some embodiments, the plasmid comprises a gene for valyl-tRNA synthesase^{ts} and the preselected organism is E. coli AB4141. In some embodiments, the plasmid has SEQ. ID. NO.: 667.

In some embodiments, the frameshift is a −1 frameshift. In other embodiments, the frameshift is a +1 frameshift. In some embodiments, the DNA insertion site comprises a restriction site.

In some embodiments, the piece of DNA is an intermediate fragment. In other embodiments, the piece of DNA is a full-length gene.

In some embodiments, the piece of DNA is isolated by polymerase chain reaction.

Some embodiments provide a frameshifted vector with SEQ. ID. NO.: 661. Other embodiments provide a frameshifted vector with SEQ. ID. NO.: 667.

Some embodiments provide a method of synthesizing a frameshifted vector and/or a frameshifted vector synthesized therewith. The method comprises at least the steps of: selecting a vector comprising an open reading frame, wherein the open reading frame comprises a functional portion that encodes a functional polypeptide, the expression of which changes the phenotype of a preselected organism transformed with the vector; and inserting and/or deleting one or more bases in the open reading frame upstream of the functional portion of the gene, thereby changing the reading frame of the functional portion of the gene, thereby producing a frameshifted vector that, when transformed into the preselected organism does not express a functional polypeptide.

In some embodiments, the inserting and/or deleting comprises cutting the vector with a restriction enzyme, digesting the overhanging single-stranded portions, and religating the vector. In some embodiments, the inserting and/or deleting comprises site-directed mutagenesis.

Some embodiments provide a kit for isolating a piece of DNA with a predetermined sequence. The kit comprises at least a frameshifted vector and instructions for isolating a piece of DNA with a predetermined sequence using the frameshifted vector. The frameshifted vector comprises an open reading frame comprising a gene comprising a functional portion that expresses a functional polypeptide, the expression of which changes the phenotype of a preselected organism transformed with the vector; and a DNA insertion site upstream of the functional portion of the gene, wherein the open reading frame comprises a frameshift upstream of the functional portion of the gene, such that the vector does not express a functional polypeptide. Some embodiments of the kit comprise a frameshifted vector with a −1 frameshift and a frameshifted vector with a +1 frameshift. Some embodiments of the kit further comprise a component selected from the group consisting of the preselected organism, a restriction enzyme, a culture medium, and combinations thereof.

### BRIEF DESCRIPTION OF THE DRAWINGS

FIG. 1A illustrates an embodiment of the disclosed method for synthesizing a DNA sequence by overlap extension in which a large piece of DNA is divided into five medium-sized pieces. FIG. 1B illustrates an embodiment for the synthesis a DNA sequence by overlap extension in which a medium-sized piece of DNA is divided into 12 short segments.

FIG. 2 illustrates an embodiment using a direct self-assembled DNA construct, from which a full-length DNA sequence is produced by ligation.

FIG. 3 illustrates an embodiment of the disclosed method for synthesizing a synthetic gene or piece of DNA.

FIG. 4A-FIG. 4C illustrate the yields of full-length oligonucleotide of length 20 to 250 nt, for coupling efficiencies of 99.5%, 99%, and 98%, respectively.

FIG. 5A schematically illustrates an embodiment comprising division and reassembly of a gene from intermediate fragments. FIG. 5B schematically illustrates an embodiment comprising the division and reassembly of one of the intermediate fragments into oligonucleotides that include leader and trailer sequences. FIG. 5C schematically illustrates an embodiment of a leader used in the expression of a polypeptide from an intermediate fragment. FIG. 5D schematically illustrates an embodiment of a trailer used in the expression of a polypeptide from an intermediate fragment.

FIG. 6 illustrates the distribution of melting temperatures for the initial codon assignment for E. coli threonine deaminase in EXAMPLE 1. (solid) Correct matches between small segment overlaps. (dash-dot) Correct matches between long strand overlaps. (dashed) Incorrect matches between small segments. (dotted) Incorrect matches between long strands.

FIG. 7 illustrates the distribution of melting temperatures after the final codon assignment for E. coli threonine deaminase in EXAMPLE 1.

FIG. 8 is the final codon assignment for E. coli threonine deaminase in EXAMPLE 1. The codon assigned to each amino acid in the protein is shown as “Xn” where “X” is a one-letter amino code and “n” is a codon index.

FIG. 9 is a key that maps an amino acid code and codon index for E. coli.

FIG. 10 illustrates the codon usage for the synthetic E. coli threonine deaminase sequence from EXAMPLE 1 vs. the codon usage in the E. coli genome. Each codon is shown as a point in which the x-coordinate is its usage per 1,000 in E. coli and the y-coordinate is its usage per 1,000 in threonine deaminase. (x, solid line) Usage in native threonine deaminase. (o, dashed line) Usage in the synthetic gene.

FIG. 11A-FIG. 11E provide an overlap map of the synthesized DNA segments of EXAMPLE 1. Each row corresponds to a short segment of single-stranded DNA that was synthesized directly. The overlaps between short segments are indicated by brackets: [ ]. The overlaps between long strands are indicated by braces: { }.

FIG. 12A-FIG. 12E illustrate the synthesized oligonucleotide segments from EXAMPLE 1 with forward and reverse complements indicated. Each row corresponds to a short segment of single-stranded DNA that was synthesized directly. Overlaps between short segments are indicated by underlining and numbering. Each underlined region in a forward segment is the reverse complement of the underlined region having the same number, and vice versa. Reverse complement region numbers are primed.

FIG. 13 is a gel of the products of the first set of overlap extension reactions in EXAMPLE 1.

FIG. 14 is a gel of the products of the PCR reactions in EXAMPLE 1.

FIG. 15 is a gel of the products of the second overlap extension reaction in EXAMPLE 1.

FIG. 16 illustrates the distribution of melting temperatures for the initial codon assignment for variola DNA polymerase-1 in EXAMPLE 2. (solid) Correct matches between small segment overlaps. (dash-dot) Correct matches between long strand overlaps. (dashed) Incorrect matches between small segments. (dotted) Incorrect matches between long strands.

FIG. 17 illustrates the distribution of melting temperatures after the final codon assignment for variola DNA polymerase-1 in EXAMPLE 2.

FIG. 18 is the final codon assignment for variola DNA polymerase-1 in EXAMPLE 2. The codon assigned to each amino acid in the protein is shown as “Xn” where “X” is a one-letter amino code and “n” is a codon index.

FIG. 19 illustrates the codon usage for the synthetic variola DNA polymerase-1 sequence from EXAMPLE 2 vs. the codon usage in the E. coli genome. Each codon is shown as a point in which the x-coordinate is its usage per 1,000 in E. coli and the y-coordinate is its usage per 1,000 in threonine deaminase. (x, solid line) Usage in native threonine deaminase. (o, dashed line) Usage in the synthetic gene.

FIG. 20 illustrates the distribution of melting temperatures at the initial codon assignment for variola DNA polymerase-2 in EXAMPLE 2. (solid) Correct matches between small segment overlaps. (dash-dot) Correct matches between long strand overlaps. (dashed) Incorrect matches between small segments. (dotted) Incorrect matches between long strands.

FIG. 21 illustrates the distribution of melting temperatures after the final codon assignment for variola DNA polymerase-2 in EXAMPLE 2.

FIG. 22 is the final codon assignment for variola DNA polymerase-2 in EXAMPLE 2. The codon assigned to each amino acid in the protein is shown as “Xn” where “X” is a one-letter amino code and “n” is a codon index.

FIG. 23 illustrates the codon usage for the synthetic variola DNA polymerase-2 sequence from EXAMPLE 2 vs. the codon usage in the E. coli genome. Each codon is shown as a point in which the x-coordinate is its usage per 1,000 in E. coli and the y-coordinate is its usage per 1,000 in threonine deaminase. (x, solid line) Usage in native threonine deaminase. (o, dashed line) Usage in the synthetic gene.

FIG. 24A-FIG. 24G are overlap maps for the synthesized DNA segments for variola polymerase-1 of EXAMPLE 2, including DNA polymerase leaders (FIG. 24A) and trailers (FIG. 24G). Each row corresponds to a short segment of single-stranded DNA that was synthesized directly. The overlaps between short segments are indicated by brackets:

FIG. 25A-FIG. 25G are overlap maps for the synthesized DNA segments for variola polymerase-2 of EXAMPLE 2, including DNA polymerase leaders (FIG. 25A) and trailers (FIG. 25G). Each row corresponds to a short segment of single-stranded DNA that was synthesized directly. The overlaps between short segments are indicated by brackets: [ ].

FIG. 26A-FIG. 26E illustrate the synthesized segments for variola polymerase-1 from EXAMPLE 2 with forward and reverse complement indicated. Each row corresponds to a short segment of single-stranded DNA that was synthesized directly. Overlaps between short segments are indicated by underlining and numbering. Each underlined region in a forward segment is the reverse complement of the underlined region having the same number, and vice versa. Reverse complement region numbers are primed.

FIG. 27A-FIG. 27E illustrate the synthesized segments for variola polymerase-2 from EXAMPLE 2 with forward and reverse complement indicated. Each row corresponds to a short segment of single-stranded DNA that was synthesized directly. Overlaps between short segments are indicated by underlining and numbering. Each underlined region in a forward segment is the reverse complement of the underlined region having the same number, and vice versa. Reverse complement region numbers are primed.

FIG. 28 is a gel of fragments 0-4, which make up variola DNA polymerase-1 from EXAMPLE 2.

FIG. 29 is a gel of fragments 5-9, which make up variola DNA polymerase-2 from EXAMPLE 2.

FIG. 30 is a gel of Parts I and II of variola DNA polymerase from EXAMPLE 2.

FIG. 31 is a gel of the ligation product of Parts I and II of variola DNA polymerase from EXAMPLE 2.

FIG. 32 illustrates the distribution of melting temperatures for the initial codon assignment for E. coli threonine deaminase in EXAMPLE 3. (solid) Correct matches between small segment overlaps. (dashed) Incorrect matches between small segments.

FIG. 33 illustrates the distribution of melting temperatures after the final codon assignment for E. coli threonine deaminase in EXAMPLE 3.

FIG. 34 is the final codon assignment for E. coli threonine deaminase in EXAMPLE 3. The codon assigned to each amino acid in the protein is shown as “Xn” where “X” is a one-letter amino code and “n” is a codon index.

FIG. 35 illustrates the codon usage for the synthetic E. coli threonine deaminase sequence from EXAMPLE 3 vs. the codon usage in the E. coli genome. Each codon is shown as a point in which the x-coordinate is its usage per 1,000 in E. coli and the y-coordinate is its usage per 1,000 in threonine deaminase. (x, solid line) Usage in native threonine deaminase. (o, dashed line) Usage in the synthetic gene.

FIG. 36 provides an overlap map for the synthesized DNA segments of EXAMPLE 3. Each row corresponds to a short segment of single-stranded DNA that was synthesized directly.

FIG. 37A-FIG. 37F illustrate the synthesized segments from EXAMPLE 3 with forward and reverse complement indicated, including leader (FIG. 37A) and trailer (FIG. 37F) primers. Each row corresponds to a short segment of single-stranded DNA that was synthesized directly. Overlaps between short segments are indicated by underlining and numbering. Each underlined region in a forward segment is the reverse complement of the underlined region having the same number, and vice versa. Reverse complement region numbers are primed.

FIG. 38 is a gel of the four Medium-sized pieces synthesized by direct self-assembly and ligation in EXAMPLE 3.

FIG. 39 is a gel of the threonine deaminase gene synthesized by ligation of the four Medium-sized pieces in EXAMPLE 3.

FIG. 40A-FIG. 40E illustrate the sequences of the gene leader, three intermediate fragments, and gene trailer for the Ty3 GAG3 ORF synthesized in EXAMPLE 4.

FIG. 41 is an agarose gel of the three intermediate fragments of the Ty3 GAG3 synthesized in EXAMPLE 4.

FIG. 42 is an agarose gel of the Ty3 GAG3 gene synthesized in EXAMPLE 4.

FIG. 43 is an SDS-PAGE gel of the Ty3 Gag3p polyprotein produced in EXAMPLE 4.

FIG. 44 illustrates the sequence for the Ty3 IN gene synthesized in EXAMPLE 5.

FIG. 45A-FIG. 45L provide overlap maps for the leader, ten intermediate fragments, and trailer for the Ty3 IN gene synthesized in EXAMPLE 5.

FIG. 46A-FIG. 46L illustrate the 50 nt oligonucleotides used in the synthesis of the intermediate fragments for the Ty3 IN gene synthesized in EXAMPLE 5.

FIG. 47 is an agarose gel of the ten intermediate fragments of the Ty3 IN gene synthesized in EXAMPLE 5.

FIG. 48 is an agarose gel of a TY3 IN gene synthesized in EXAMPLE 5.

FIG. 49 is an SDS-PAGE gel of a TY3 IN protein synthesized in EXAMPLE 5.

FIG. 50 is a scheme illustrating the synthesis and use of an α-fragment frameshifted vector as described herein.

FIG. 51 is a schematic diagram of the pGEM®-3Z vector.

FIG. 52 is the sequence of the reverse complement of the α-fragment frameshifted vector described herein. The gene for the α-complementing fragment is in bold.

FIG. 53 is a scheme illustrating the synthesis and use of the pCG2 frameshifted vector as described herein.

### DETAILED DESCRIPTION OF THE PREFERRED EMBODIMENT

As used herein, the term “DNA” includes both single-stranded and doubled-stranded DNA. The term “piece” may refer to either a real or hypothetical piece of DNA depending on context. A “very large” piece of DNA is longer than about 1,500 bases, a “large” piece of DNA is about 1,500 bases or fewer, a “medium-sized” piece of DNA is about 300 to 350 bases or fewer, and a “short” piece of DNA is about 50 to 60 bases or fewer. Short pieces of DNA are also referred to as “oligonucleotides” by those skilled in the art. It will be appreciated that these numbers are approximate, however, and may vary with different processes or process variations. Although descriptions of preferred embodiments of the disclosed method that follow describe each recursive or hierarchical step as involving pieces of DNA of the same size range—for example, in which all of the pieces of DNA are very large, large, medium-sized, or short—one skilled in the art will appreciate a hierarchical step may involve DNA from more than one size range. A particular step may involve both short and medium-sized pieces of DNA, or even short, medium-sized, and large pieces of DNA.

A “small” or “short” piece of DNA is a DNA segment that can be synthesized, purchased, or is otherwise readily obtained. The term “segment” is also used herein to mean “small piece.” Those skilled in the art will understand that the term “synthon” is synonymous with the terms “small piece” and “segment” as used herein, although the term “synthon” is not used herein. The DNA segments used in the Examples that follow are synthetic; however, the disclosed method also comprehends using DNA segments derived from other sources known in the art, for example, from natural sources including viruses, bacteria, fingi, plants, or animals; from transformed cells; from tissue cultures; by cloning; or by PCR amplification of a naturally occurring or engineered sequence. As used herein, a “correct” piece of DNA is a piece of DNA with the correct or desired nucleotide sequence. An “incorrect” piece is one with an incorrect or undesired nucleotide sequence.

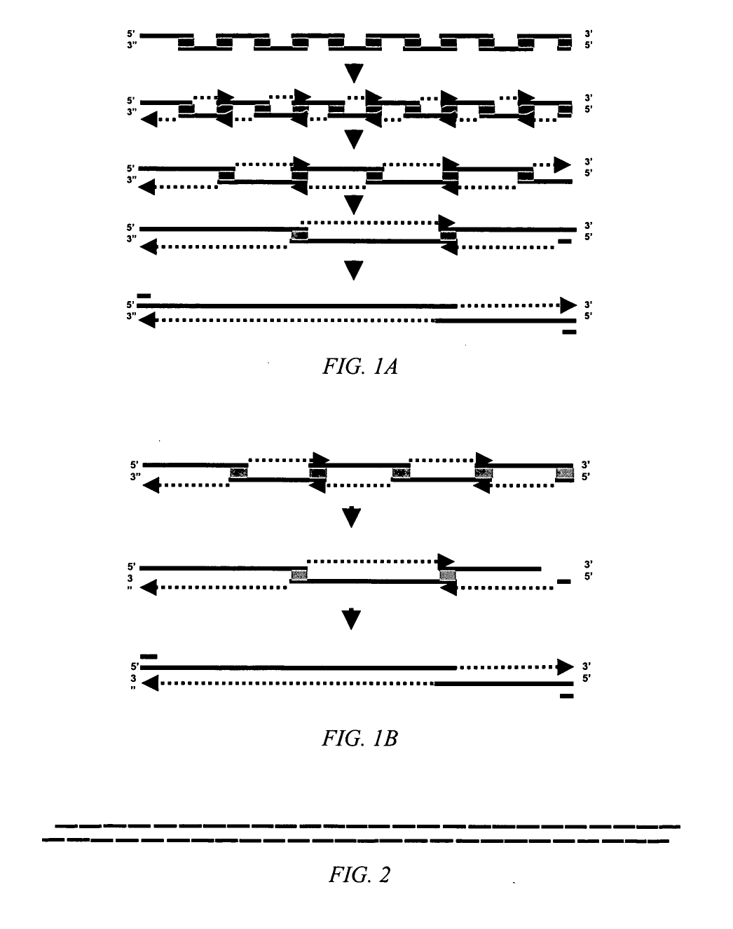

The disclosed method proceeds by a divide-and-conquer strategy (See Aho et al. The Design and Analysis of Computer Algorithms Addison-Wesley; Reading, Mass.: 1974, the disclosure of which is incorporated by reference in its entirety). A problem that is too large to be solved directly is broken recursively into smaller sub-problems until each is small enough to be solved directly, then the small sub-solutions are combined recursively into a solution to the original problem. Here, a particular full-length gene is too long to synthesize directly. It is broken recursively into smaller overlapping pieces until each is small enough to be synthesized. The gene is then reassembled in the reverse order of the disassembly. The smallest pieces are reassembled into the next larger pieces, which are reassembled into the next larger pieces, and so on and so forth. A reassembly step may be performed by overlap extension using a high-fidelity DNA polymerase as illustrated in FIG. 1A and FIG. 1B, or by ligation as illustrated in FIG. 2. Those skilled in the art will realize that ligation may also be used to reassemble a single strand of DNA using a variation of the DNA construct illustrated in FIG. 2 in which the pieces of DNA that comprise one strand abut, but all of the pieces of DNA comprising the complementary strand do not. Another method for reassembly is cloning into an expression vector and transformation of an appropriate host. Those skilled in the art will understand that many methods of cloning are compatible with the disclosed method, for example, exonuclease III cloning, topoisomerase cloning, restriction enzyme cloning, and homologous recombination cloning.

As discussed in greater detail below, a synthetic oligonucleotide is typically a mixture containing the desired oligonucleotide mixed with incorrect oligonucleotides, that is, oligonucleotides that do not have the desired sequence. As would be apparent to one skilled in the art, synthesizing a gene from such a mixture will likely produce the correct or desired gene in admixture with incorrect genes. One method for synthesizing only the correct gene is to reassemble the gene from pieces of DNA that are likely to have the correct sequences. Consequently, in some embodiments, during the reassembly process, pieces of DNA are selected that are likely to have the correct sequences for use in subsequent reassembly steps. In some embodiments, the criterion for the selection is a property of a reassembled piece of DNA or a polypeptide encoded by and expressed therefrom. In some embodiments, the criterion for the selection is a property determined from the full-length piece of DNA or polypeptide expressed therefrom. In some embodiments, the criterion for the selection is a property determined for the complementary strand of DNA or polypeptide expressed therefrom. In some embodiments, the criterion for the selection is a property determined for a piece of RNA transcribed from the piece of DNA. In some embodiments, the criterion for the selection is the phenotype of an organism into which the DNA is inserted.

Any property indicative of the correctness of the DNA sequence is useful in the method. The likelihood or probability of selecting a correct sequence depends on the property that is measured or determined. In some embodiments, the likelihood approaches certainty. An example of such a property is an experimentally determined nucleotide sequence of the piece of DNA. Any method known in the art for determining a nucleotide sequence may be used, including automated sequencing, manual sequencing, mass spectroscopic sequencing, and the like. In other embodiments, the property indicates that the piece of DNA probably has the correct sequence, but does not confirm the correctness of the sequence. An example of such a property is the molecular weight of a polypeptide encoded by and expressed from the piece of DNA. Any method known in the art for determining the molecular weight of a polypeptide or protein may be used, including gel electrophoresis, mass spectroscopy, and the like.

The term “PCR” as used herein in the context of assembling or reassembling DNA is a PCR or overlap extension reaction, preferably using a proof-reading DNA polymerase (“proof-reading PCR”). The term “direct self-assembly” as used herein in the context of assembling or reassembling DNA is a copy-free method of producing a DNA construct or a DNA construct produced by the method, comprising assembling a large piece of DNA from short synthetic segments in a single step. “Copy-free” means that the method lacks a copy step, such as is found in overlap extension or PCR, thus eliminating the copying errors. In a preferred embodiment, adjacent segments on the same strand abut, i.e., form a nick in the strand. Preferably, the nicks in the self-assembly are repaired by in vitro ligation. In another preferred embodiment, the nicks are repaired in vivo by cellular machinery after cloning.

The method comprises (a) hierarchical assembly (b) by high-fidelity techniques such as overlap extension using proof-reading DNA polymerase, ligation, cloning, or other methods, (c) with optimization of the sequences in the component oligonucleotides to facilitate preferential hybridization to the desired adjacent piece(s) and to disfavor undesired hybridizations to other pieces, for example, by exploiting the degeneracy of the genetic code or a regulatory region consensus sequence, (d) to achieve a DNA melting temperature gap between correct (high melting temperature) and incorrect (low melting temperature) hybridizations, (e) optionally selecting pieces of DNA likely to have the correct DNA sequence for use in the subsequent assembly steps, (f) so that, with high probability correct assemblies will form.

Hierarchical assembly ensures that the complexity at any step, and therefore the possible number of incorrect assemblies, is bounded and manageably small. Overlap extension allows every correctly hybridized 3′ end to be extended reliably to a complementary copy of the prefix of its match. Using a high-fidelity DNA polymerase reaction ensures that the copy, with high probability, is correct. Ligation and cloning achieve the same result as overlap extension while avoiding the small but non-zero error rate associated with DNA polymerase reactions. The genetic code degeneracy permits flexibility in silent codon substitutions to strengthen correct matches and disrupt incorrect ones. A broad temperature gap between the highest-melting incorrect hybridization and the lowest-melting correct hybridization means that, with high probability, most incorrect ones have melted and most correct ones have annealed. Selecting gene fragments likely to be correct means that oligonucleotide sequence errors arising from chemical synthesis will be removed or reduced. Consequently, each reassembly reaction, with high probability, produces a correct larger piece of DNA. Errors will occur, of course, but with low probability. Consequently, two or more compensating errors that yield a product of the same molecular weight—i.e., the same band in the final gel—or two or more compensating deletions that provide a product of the same reading frame—i.e., the same or nearly the same encoded amino acid sequence—would correspond to a doubly rare or rarer event.

In optimizing the base sequences, theoretical melting temperatures are calculated for all possible correct and incorrect hybridizations by methods known in the art, for example, using Mfold. Such methods are disclosed, for example, in M. Zuker et al. “Algorithms and thermodynamics for RNA secondary structure prediction: A practical guide.” in RNA Biochemistry and Biotechnology, Barciszewsld & Clark, eds.; Kluwer: 1999; D. H. Mathews et al. “Expanded sequence dependence of thermodynamic parameters provides robust prediction of RNA secondary structure” J. Mol. Biol., 1999, 288, 910-940; J. Santa-Lucia “A unified view of polymer, dumbbell, and oligonucleotide DNA nearest-neighbor thermodynamics” Proc. Natl. Acad. Sci. USA, 1998, 95, 1460-1465; the disclosures all of which are incorporated by reference in their entireties. The figure of merit is the gap between the lowest-melting correct match and the highest-melting incorrect match. Examples of incorrect matches include: (a) hairpins, in which a short segment folds back and hybridizes to itself; (b) dimers, in which a short segment is partially self-complementary; (c) intersegment mismatches, in which part of one short segment is partially complementary to part of a second; (d) and shifted correct matches, in which a misaligned overlap region is partially complementary to another region within the same overlap. Accordingly, in some embodiments, optimization comprises calculating a melting temperature for a single piece of DNA, for example, for a hairpin, in some embodiments, optimization comprises calculating a melting temperature for two pieces of DNA, for example, for an intersegment mismatch. In some embodiments, optimization comprises calculating both types of melting temperatures.

The melting temperature gap is widened by perturbations to the codon assignments, including strengthening correct matches by increasing G-C content in the overlaps and disrupting incorrect matches by choosing non-complementary bases. Codon assignments are varied and the process repeated until the gap is comfortably wide. This process may be performed manually or automated. In a preferred embodiment, the search of possible codon assignments is mapped into an anytime branch and bound algorithm developed for biological applications, which is described in R. H. Lathrop et al. “Multi-Queue Branch-and-Bound Algorithm for Anytime Optimal Search with Biological Applications” in Proc. Intl. Conf. on Genome Informatics, Tokyo, Dec. 17-19, 2001 pp. 73-82; in Genome Informatics 2001 (Genome Informatics Series No. 12), Universal Academy Press, the disclosure of which is incorporated by reference. Those skilled in the art will recognize that other optimization methods could be used, e.g., simulated annealing, genetic algorithms, other branch and bound techniques, hill-climbing, Monte Carlo methods, other search strategies, and the like. Those skilled in the art will further realize that optimizing, i.e., weakening, incorrect matches is functionally equivalent to optimizing, i.e., strengthening, correct matches, and vice versa. Consequently, suitable optimization methods include weakening incorrect matches, strengthening correct matches, and any combination thereof.

Those skilled in the art will realize that the size of the melting temperature gap is related to the annealing conditions such that a narrower gap may require more stringent annealing conditions in the reassembly step to provide the requisite level of fidelity. Consequently, the temperature gap has no minimum value. In some embodiments, the temperature gap is greater than 0° C., at least about 1° C., at least about 2° C., at least about 3° C., at least about 4° C., at least about 5° C., at least about 6° C., at least about 7° C., at least about 8° C., at least about 9° C., at least about 10° C., at least about 12° C., at least about 14° C., at least about 16° C., at least about 18° C., or at least about 20° C. Those skilled in the art will understand that, under appropriate annealing conditions, the temperature gap is arbitrarily close to 0° C. Practically, the difference between the lowest-melting correct match and the highest melting incorrect match is at least about 1° C., more preferably, at least about 4° C., more preferably, at least about 8° C., most preferably, at least about 16° C. The wider the temperature gap, the more robust the self-assembly, thereby permitting the use of less stringent annealing conditions.

Those skilled in the art will realize that the temperature gap may be increased by optimizing the division process. For example, the division may be optimized through a nested search strategy, described below. Other appropriate search strategies will be apparent to those skilled in the art.

In an outer search, a temperature variable is initialized to a high temperature, for example, 80° C., then decremented by, for example, one degree at each outer search step. At each outer search step, an inner search is called to see whether it is possible to divide the gene into short pieces such that no overlap region has a melting temperature lower than the current setting of the temperature variable. When the inner search finally succeeds, its corresponding codon assignments are returned from the outer search.

The inner search proceeds via a depth-first search through the possible division points that meet the design constraints, for example, minimum overlap lengths, maximum segment lengths, possible C-G clamps at segment boundaries, and the like. At each inner search step, the overlap region resulting from the most recent candidate division point is used to generate the set of codon assignments that yield the highest self melting temperature for that region. If the highest self-melting temperature is less than the current setting of the temperature variable the inner search fails. Otherwise it continues to the next step.

While the general DNA melting problem is non-linear because DNA secondary structure can cause non-local elements of the sequence to hybridize, correct matches at the overlap regions are characterized by linear hybridizations among purely local bases. Consequently, for a given subsequence of the gene, linear dynamic programming techniques based on base pairs (nearest neighbors) are used to determine the codon assignment to that subsequence that maximizes its melting temperature.

Thus, the gene is divided into short pieces by finding the division points that maximize the melting temperature of the lowest-melting overlap region, while respecting the design constraints, for example, minimum overlap lengths, maximum segment lengths, possible C-G clamps at segment boundaries, and the like. The codon assignments corresponding to the maximal division points are a better starting codon assignment at the beginning of the design process than are the most common codons.

Those skilled in the art will appreciate that optimization may be performed using other parameters or measures related to hybridization propensity, for example, free energy, enthalpy, entropy, or other arithmetic or algebraic combinations of such parameters or measures, to achieve the same effect as melting temperature. Melting temperature itself is one such arithmetic or algebraic combination of such parameters or measures.

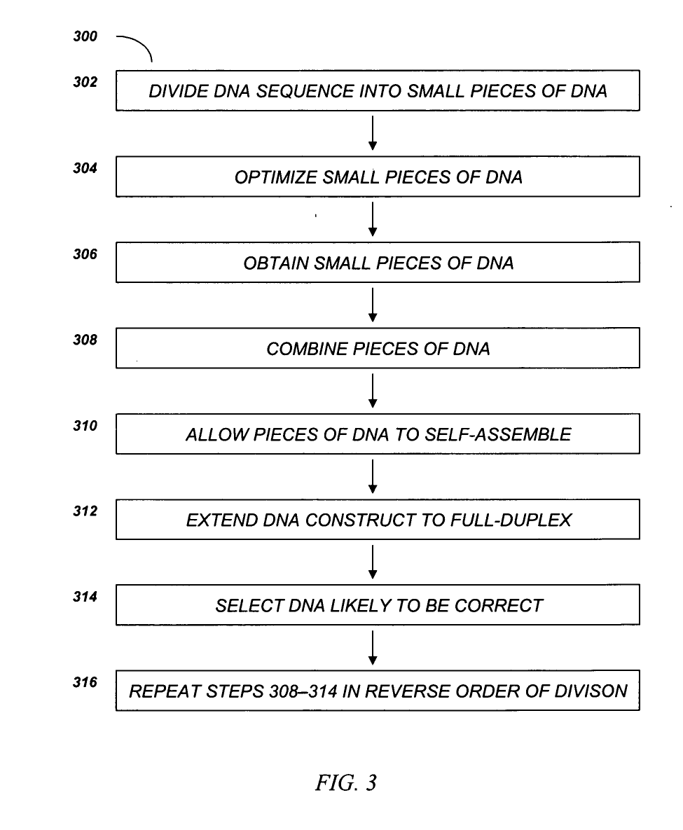

The disclosed method comprises a design or decomposition process and a synthesis or reassembly process. In a preferred embodiment, the synthetic gene is designed according to a method illustrated as method 300 in FIG. 3. In step 302, the DNA sequence or gene is divided into small pieces of DNA or oligonucleotides. In step 304, the small pieces of DNA are optimized. In step 306, the optimized small pieces of DNA are obtained. In step 308, the pieces of DNA derived from one division of each piece of DNA are combined. In step 310, the pieces of DNA are allowed to self-assemble into a DNA construct. In step 312, the DNA construct is extended to full-duplex DNA. In step 314, a property indicative of the likelihood of the correctness of the resulting piece of DNA is determined, and pieces of DNA that are likely to have the correct sequence selected. In step 316, steps 308-314 are repeated in reverse order of the division in step 312 to produce the synthetic gene.

Step 302. The synthetic gene is divided as follows. If the synthetic gene is very large, the DNA sequence is optionally divided into two or more large pieces of DNA of roughly equal size. Adjacent pieces of DNA preferably overlap by a number of nucleotides appropriate to facilitate reassembly. The extent of overlap depends on factors including the particular base sequence, method of reassembly, temperature, and salt concentration, and may be determined by the skilled artisan without undue experimentation. The adjacent pieces of DNA are designed for reassembly by any method or combination of methods known in the art for joining DNA molecules, for example, by ligation or by overlap extension. In some embodiments, the division is optimized to produce pieces of DNA that are more likely to assemble into the desired DNA sequence, as described in greater detail below.

In a preferred embodiment, each large piece is dsDNA and overlaps the adjacent large piece by at least the width of a restriction site (typically from about four to about six bases). Preferably, a restriction site that does not appear elsewhere in either piece is engineered into the DNA sequence of each resulting piece by exploiting the degeneracy of the genetic code as described in greater detail below. Adjacent large pieces are reassembled by cutting with the appropriate restriction enzyme, annealing the adjacent pieces together, and ligating the cut ends together.

In other embodiments, the large pieces of ssDNA or dsDNA are designed such that adjacent pieces of DNA on the same strand abut without a gap, as illustrated in FIG. 2. The DNA is reassembled by annealing and ligation. In a preferred embodiment, the DNA is single-stranded. In some embodiments, the large pieces of DNA are about 3000 bases long. In some embodiments, adjacent large pieces of DNA overlap by about 1500 bases.

In another preferred embodiment, each large piece is ssDNA or dsDNA and directly abuts the adjacent large piece. In this embodiment, primers are then constructed that overlap the abutting ends of the large pieces by from about 25 to about 30 bases or greater, and the abutting large pieces are reassembled by overlap extension.

In yet another preferred embodiment, each large piece is dsDNA with overlapping regions comprising from about 25 to about 30 bases or greater of complementary ssDNA. In this embodiment, the adjacent large pieces are reassembled by hybridization and ligation. In another embodiment, the ssDNA overlaps are designed to leave single-stranded regions flanking the double-stranded overlap region. In this embodiment, the adjacent large pieces are reassembled by overlap extension.

In still another embodiment, each resulting piece is ssDNA and the overlapping regions of adjacent resulting pieces comprise from about 25 to about 33 bases or greater of complementary single-stranded DNA. In some preferred embodiments, the overlapping regions comprise about 75 bases. In this embodiment, the adjacent resulting pieces are reassembled by overlap extension.

In certain embodiments, the large or very large pieces of DNA may be reassembled by cloning using a vector of any type known in the art. Examples of suitable vectors include without limitation, plasmids, cosmids, phagemids, viruses, chromosomes, bacterial artificial chromosomes (BAC), or synthetic chromosomes.

Embodiments using ligation or cloning are preferred in situations in which using DNA polymerase is disfavored, for example, where the DNA is greater than about 3 KB to about 5 KB long.

The method for designing a piece of DNA is described for a large piece of DNA, but is applicable to pieces of DNA of any size. The piece of DNA is divided into overlapping short pieces of DNA that are readily available—that is, small pieces or segments. Preferably, each short piece is small enough to be synthesized readily. This large piece of DNA could be the target DNA sequence or a large piece of DNA derived from the division of a very large piece of DNA as described above.

In a preferred embodiment, the large piece of DNA is designed for “direct self-assembly.” In this embodiment, each large piece is divided into from about 50 to about 60 overlapping small pieces of from about 50 to about 60 bases or fewer. Preferably, the adjacent small pieces of DNA from the same strand abut, i.e., hybridize to form a DNA construct with no gaps between the pieces. In this embodiment, the large piece of DNA is preferably reassembled by ligation (“direct self-assembly and ligation”). In an embodiment in which adjacent small pieces from the same strand do not abut, i.e., hybridize to form a DNA construct with single-stranded gaps between the double-stranded overlaps, the large piece of DNA is preferably reassembled by overlap extension. In another embodiment, adjacent small pieces from the same strand abut, i.e., hybridize to form a DNA construct with no single-stranded gaps between the double-stranded overlaps, and the large piece of DNA is reassembled by overlap extension. In another embodiment, a DNA construct with a combination of gaps and no gaps is reassembled by overlap extension. In another preferred embodiment, the large piece of DNA is reassembled by cloning in an expression vector. In this embodiment, the ends of the DNA construct may have any combination of gaps and no gaps. Preferably, the ends of the large piece of DNA are adapted for insertion into an expression vector, for example, complementary to a restriction site in the expression vector.

In another preferred embodiment, the large piece of DNA is designed for “recursive assembly” or “hierarchical assembly.” In this embodiment, a large piece of DNA is divided first into about overlapping medium-sized pieces of DNA. In some embodiments, the large piece of DNA is divided into about three to about ten medium-sized pieces of DNA, preferably, about five to about 7 pieces. Each medium-sized piece is then subdivided into overlapping small pieces of DNA, preferably, from about six to about 12 pieces. As described above for direct self-assembly, the DNA pieces at each level of recursion may be designed for reassembly by any combination of methods, including ligation, overlap extension, or cloning. In a preferred embodiment, the DNA pieces are reassembled by overlap extension. One skilled in the art will realize that direct self-assembly is a special case of recursive assembly in which the large piece of DNA is divided in a single step.

Step 304. For the resulting pieces of DNA at each level of recursion, the sequences are optimized to strengthen correct matches (the overlap regions between adjacent pieces of DNA) and to disrupt incorrect matches (all other hybridizations). For example, a DNA sequence in a coding region may be optimized by taking advantage of the genetic code degeneracy. A DNA sequence in a regulatory region may be optimized by taking advantage of the degeneracy in the regulatory region consensus sequence. A DNA sequence outside a coding or regulatory region, i.e., in an intergenic region, may be optimized by direct base assignment.

Some embodiments use no or limited sequence optimization. For example, changes in a nucleotide sequence can change the secondary structure of DNA and RNA. Changes in the secondary structure in RNA viral genomes can affect the viability of the viruses. In some embodiments, no sequence optimization is performed. In other embodiments, selected sequences are optimized as described above and other sequences are not.

In some embodiments, the division described in step 302 is optimized to increase the probability that the pieces of DNA will reassemble into the desired DNA sequence. The boundary points between adjacent pieces of DNA are adjusted to create or to increase a temperature gap, or to disrupt other incorrect hybridizations, for example, hairpins.

Reassembly or synthesis of the synthetic gene is the formal reverse of the division process described in steps 302 and 304.

Step 306. Obtain the optimized small pieces of DNA. Typically, the small pieces of DNA are synthetic. In a preferred embodiment, the small pieces of DNA are single-stranded and overlapping portions of adjacent pieces are complementary.

Step 308. Combine the pieces of DNA derived from one division of each piece of DNA. In a recursive assembly process, the small pieces derived from each medium-sized piece are combined in this step. In the next recursive assembly cycle, the resulting medium-sized pieces derived from each large piece are combined in this step, and so on and so forth. In a direct self-assembly process, the small pieces derived from the large piece of DNA are combined in this step.

Step 310. Allow the DNA segments to self-assemble- to form a DNA construct of ssDNA segments connected by double-stranded overlap regions. In embodiments in which pieces of DNA are double-stranded, the pieces are preferably first denatured. Embodiments using overlap extension to reassemble a piece of DNA have single-stranded gaps between the double-stranded overlap regions. Preferably, the single-stranded gaps are from about zero to about 20 bases long. Embodiments using ligation to reassemble a piece of DNA have single-stranded gaps of length zero (i.e., no gap, a nick in the DNA) and the double-stranded overlap regions abut each other. Embodiments using cloning to reassemble a piece of DNA have any combination of gaps and no gaps.

Step 312. Extend the DNA construct to full-duplex dsDNA. In embodiments with single-stranded gaps between the double-stranded overlaps, extension is accomplished using overlap extension, preferably, using a high-fidelity DNA polymerase reaction. In embodiments with no gaps between the double-stranded overlaps, extension is accomplished by ligation. In another preferred embodiment, the self-assembled construct is cloned into an expression vector, and the extension to full-duplex dsDNA is performed by the cellular machinery.

Some embodiments use ssDNA in subsequent steps. ssDNA is produced from the dsDNA using any method known in the art, for example, by denaturing or using nicking enzymes. In some embodiments, the DNA is cloned into a vector that produces ssDNA, for example, bacteriophage M13 or a plasmid containing the M13 origin of DNA replication. M13 is known to roll-off ssDNA into the medium.

Step 314. In some embodiments, a property indicative of the likelihood of correctness of the resulting piece of DNA is optionally determined as disclosed in greater detail below. Pieces of DNA likely to have the correct sequence are selected for subsequent reassembly steps. In some embodiments, the property is determined after the synthetic gene is fully reassembled in order to select a synthetic gene likely to have the correct sequence. In some embodiments, the sequence of the selected synthetic gene is confirmed by sequencing.

Step 316. Repeat steps 308-314 in reverse order of the division in step 312 to produce the synthetic gene. Those skilled in the art will appreciate that the pieces of DNA identified in the division process may each be synthesized by a different reassembly method. For example, one piece may be synthesized by overlap extension, while a second is synthesized by ligation, and these two pieces reassembled by cloning into an expression vector.

In a preferred embodiment, the disclosed method takes advantage of the fact that the genetic code is sufficiently degenerate to allow codons to be assigned so that, with high probability, wrong hybridizations melt at lower temperatures and correct hybridizations melt at higher temperatures. Consequently, there is an intermediate temperature range within which, with high probability, the product that does form is mostly correct. Because errors occur with low probability, two or more compensating errors that yield a product with the correct molecular weight—i.e., the same band in the final gel—or two or more compensating deletions that yield a product of the same reading frame—i.e., the same or nearly the same encoded amino acid sequence—would correspond to a doubly rare or rarer event.

The final primers, or intermediate primers, or other flanking sequences may contain sequences that allow the synthesized gene to be inserted into an expression vector using various well-known cloning methods, for example, restriction enzymes, homologous recombination, exonuclease cloning, or other methods known in the art, allowing one to build a large synthetic gene quickly and easily.

A problem in some embodiments of the disclosed method is that a synthetic oligonucleotide, or small piece of DNA, typically contains a mixture of the desired DNA sequence (“full-length oligonucleotide”) contaminated with sequences with internal point deletions. This problem is referred to herein as the “N−1” problem because oligonucleotides with a single point deletion (“N−1 oligonucleotides”) are the most common contaminant in a typical chemical synthesis of oligonucleotides. Furthermore, in some embodiments, the N−1 oligonucleotides are the most problematic because they are more likely to hybridize, and consequently, to provide undesired products, than oligonucleotides with more than one point deletion or mutation. When this mixture of oligonucleotides is used to synthesize medium and large pieces of DNA as disclosed herein, the product pieces of DNA contain a population containing DNA with the desired sequence as well as DNA with errors arising from incorporation of the N−1 oligonucleotides. The N−1 oligonucleotide errors are cumulative and may cause frame-shift mutations, as understood by those skilled in the art.

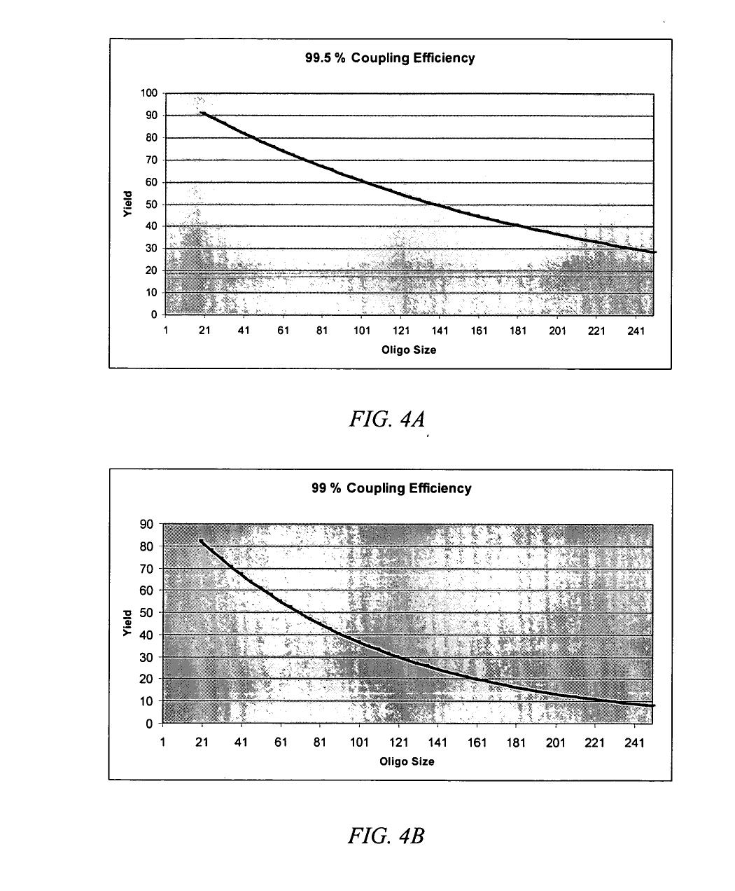

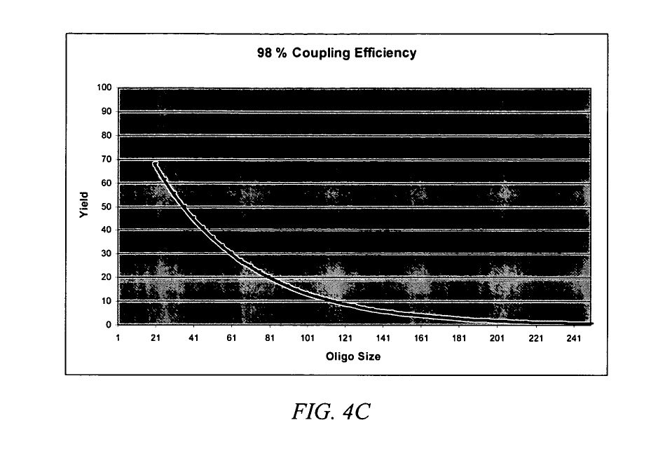

In the chemically synthesized oligonucleotides, the typical coupling efficiency for each nucleotide is from about 98% to about 99.5%, or greater. TABLE I provides the yield of the desired full-length oligonucleotide of length 20 to 250 nt for coupling efficiencies of 99.5%, 99%, and 98%. These results are provided graphically in FIG. 4A-FIG. 4C, respectively. As expected, the probability of synthesizing a correct oligonucleotide decreases with oligonucleotide length and coupling efficiency. Because each of the oligonucleotide pieces used in the construction of the synthetic gene contains some N−1 contaminant, the probability of synthesizing the desired synthetic gene decreases with the length of the synthetic gene.

TABLE I

| | Coupling Efficiency | | |
|---|---|---|---|
| Oligonucleotide Length (nt) | 99.5% | 99% | 98% |
| 20 | 90.916 | 82.617 | 68.123 |
| 25 | 88.665 | 78.568 | 61.578 |
| 30 | 86.471 | 74.717 | 55.662 |
| 35 | 84.311 | 71.055 | 50.314 |
| 40 | 82.243 | 67.573 | 45.480 |
| 45 | 80.208 | 64.261 | 41.11 |
| 50 | 78.222 | 61.112 | 37.16 |
| 55 | 76.286 | 58.117 | 33.59 |
| 60 | 74.398 | 55.268 | 30.363 |
| 65 | 72.557 | 52.56 | 27.445 |
| 70 | 70.761 | 49.984 | 24.808 |
| 75 | 69.009 | 47.534 | 22.425 |
| 80 | 67.301 | 45.204 | 20.27 |
| 85 | 65.635 | 42.989 | 18.323 |
| 90 | 64.011 | 40.882 | 16.562 |
| 95 | 62.427 | 38.878 | 14.971 |
| 100 | 60.881 | 36.973 | 13.533 |
| 110 | 57.905 | 33.438 | 11.057 |
| 115 | 56.472 | 31.799 | 9.995 |
| 120 | 55.074 | 30.240 | 9.034 |
| 130 | 52.381 | 27.239 | 7.382 |
| 140 | 49.821 | 24.734 | 6.031 |
| 150 | 47.385 | 22.369 | 4.928 |
| 160 | 45.068 | 20.23 | 4.027 |
| 170 | 42.865 | 18.296 | 3.29 |
| 180 | 40.769 | 16.546 | 2.688 |
| 190 | 38.776 | 14.964 | 2.196 |
| 200 | 36.88 | 13.533 | 1.795 |
| 210 | 35.08 | 12.24 | 1.47 |
| 220 | 33.36 | 11.07 | 1.19 |
| 230 | 31.73 | 10.01 | 0.98 |
| 240 | 30.18 | 9.05 | 0.8 |
| 250 | 28.7 | 8.19 | 0.65 |

Even in cases in which the desired synthetic gene is synthesized with high probability of correct oligonucleotide order, the desired gene is invariably mixed with many defective genes arising from N−1 oligonucleotides. In many applications, this mixture of correct and defective genes is undesirable. Accordingly, disclosed below is a method for improving the probability of synthesizing the desired gene and/or selecting the desired gene from this mixture.

In some embodiments, the N−1 problem is addressed by assembling the chemically synthesized oligonucleotides using direct self-assembly and ligation, as described above and illustrated in FIG. 2. In embodiments using direct self-assembly and ligation, all of the nucleotides in each oligonucleotide are hybridized, thereby reducing the probability that an N−1 oligonucleotide will be incorporated in the preligation DNA construct. In embodiments using overlap extension, a preextension DNA construct incorporating an oligonucleotide with a deletion in a single stranded region is about as likely as a DNA construct incorporating a correct oligonucleotide. The single-base deletion error rate in double-stranded regions is about 0.3%, while the error rate in single-stranded regions is about 0.5%.

In some embodiments, the N−1 problem is addressed by sampling the population of synthetic DNA molecules and sequencing the sampled molecules. In some embodiments, a random sample from the population of different DNA molecules produced in any of the reassembly steps, including the final step, is sequenced and only those molecules with the correct nucleotide sequence are used in the next reassembly step. The optimum sample size is related to the probability of synthesizing the desired DNA molecule. For example, a synthesis of a 200-nt oligonucleotide or intermediate fragment with a 99.5% coupling efficiency provides about 37% of the correct oligonucleotide. Randomly selecting four oligonucleotides or intermediate fragments from the product mixture provides about an 84% chance of selecting at least one correct oligonucleotide. For a 300 nt oligonucleotide or intermediate fragment at 99.5% coupling efficiency, the correct oligonucleotide makes up about 22% of the product. The probability of selecting at least one correct oligonucleotide or intermediate fragment from a sample of four oligonucleotides from this mixture is about 63%. The probabilities of selecting at least one correct oligonucleotide or intermediate fragment using sample sizes of 1, 4, 6, and 8 for syntheses with coupling efficiencies of 99.5% and 99.7% and oligonucleotide lengths of 250 nt, 300 nt, and 300 nt are provided in TABLE II. As shown in TABLE II, only a modest amount of sequencing is necessary to provide a good probability of selecting a correct oligonucleotide or intermediate fragment.

TABLE II

| | Coupling | Sample Size | | | |
|---|---|---|---|---|---|
| Oligonucleotide Length (nt) | Efficiency | 1 | 4 | 6 | 8 |
| 200 | 99.5% | 36.7 | 83.9 | 93.6 | 97.4 |
| | 99.7% | 53.7 | 95.4 | 99.0 | 99.7 |
| 250 | 99.5% | 28.6 | 74.0 | 86.7 | 93.2 |
| | 99.7% | 46.0 | 91.5 | 97.5 | 99.3 |
| 300 | 99.5% | 22.2 | 63.4 | 77.9 | 86.6 |
| | 99.7% | 39.4 | 86.5 | 95.0 | 98.2 |

In some embodiments, sampling is performed by cloning the DNA-to-be-sequenced into a suitable vector. Typically, each transformed colony corresponds to one molecule of the synthetic DNA. In some embodiments, a sample of transformed colonies are selected, the DNA sequenced, and DNA with the correct sequence is used in the next hierarchical stage of assembly. The cloning is any type of cloning known in the art. In one embodiment, the cloning is topoisomerase I (TOPO®, Invitrogen) cloning.

The sampling is performed at any of the hierarchical reassembly stages. For example, in some embodiments, an oligonucleotide is sequenced after chemical synthesis. In some embodiments, oligonucleotides or intermediate fragments are assembled into a medium-sized piece of DNA, which is then sequenced. In some embodiments, medium-sized pieces of DNA are assembled into a large piece of DNA, which is then sequenced. In some embodiments, the sampling and sequencing are performed on the medium- or large-sized pieces of DNA, which are synthesized by direct self-assembly and ligation.

In some embodiments, the N−1 problem is addressed by analyzing the polypeptide(s) expressed from a sample from the population of synthetic DNA sequences. The DNA is expressed using any means known in the art, for example, inserting the gene in an expression vector or using a cell-free expression system. In some embodiments, an organism is transformed by electroporation or using a gene gun. In some embodiments, the DNA sequence is cloned in an expression vector and expressed. As discussed above, each clone typically corresponds to one DNA molecule from the population. In some embodiments, the DNA is the full-length synthetic gene. In other embodiments, the DNA is an intermediate fragment. In the case of an intermediate fragment, those skilled in the art will realize that, in some embodiments, the intermediate fragment is designed with (1) a leader that provides a start codon in the correct reading frame, that is, provides an ATG in the DNA and a 0-2 nt filler that adjusts the reading frame in order to express the desired polypeptide, and (2) a trailer that provides one or more stop codons (TAA, TAG, or TGA) in the DNA and a 0-2 nt filler that adjusts the reading frame in order to terminate the desired polypeptide. Typically, the reading frame is the same as for the full-length synthetic gene, although other reading frames are used in some embodiments. Typically, from zero to two bases are inserted into the leader and trailer for adjusting the reading frame. Those skilled in the art will recognize that more than two bases could be used to adjust the reading frame. For example, in some embodiments, the leader and/or trailer encodes additional amino acids, restriction sites, or control sequences. Those skilled in the art will further realize that, in some embodiments, different leaders and/or trailers are used in conjunction with the same piece of DNA in different steps of the method. For example, in some embodiments, the leader and/or trailer used in the expression of a polypeptide from a piece of DNA is different from the leader and/or trailer used in the assembly of that piece of DNA. Some embodiments, provide one or more stop codons downstream (3′) of the gene in order to stop the translation of DNA fragments constructed from one or more N−1 oligonucleotides. In some embodiments, the stop codons are engineered into the expression vector. Some embodiments include at least three stop codons downstream (3′) of the gene, at least one of each in each of the three possible reading frames. Some embodiments use groups of stop codons instead of single stop codons in each reading frame.

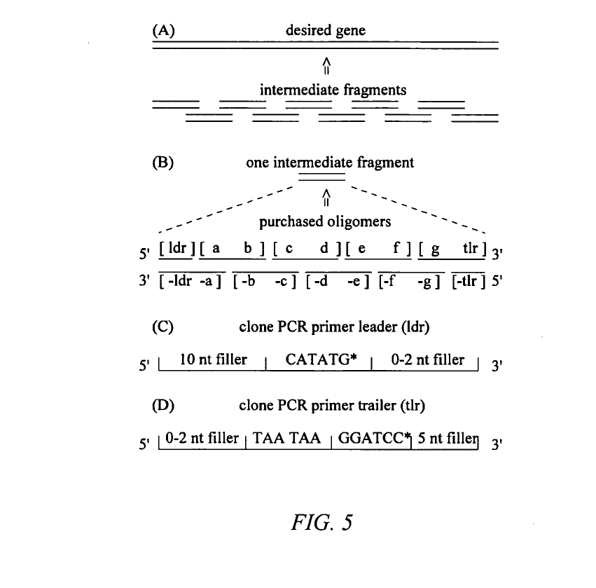

FIG. 5A-FIG. 5D illustrate an embodiment of the disclosed method in which a polypeptide is expressed from an intermediate fragment in the construction of a synthetic gene. FIG. 5A illustrates schematically the division and construction of a gene into a plurality of intermediate fragments.

FIG. 5B illustrates the division and construction of one of the intermediate fragments. The letters a-g each represents a portion of the sequence of the intermediate fragment. The brackets group these portions into oligonucleotides that are purchased or synthesized. “ldr” and “tlr” represent a leader and trailer, respectively. The corresponding portions of the sequence on the complementary strand are prefixed with a hyphen (−), i.e., “-ldr,” “-a,” . . . “-g,” and “-tlr.” Again, brackets are used to indicate the oligonucleotides.

FIG. 5C is a schematic of leader (ldr) portion illustrated in FIG. 5B. From the 5′-end, the leader comprises a 10-nt filler, a CATATG restriction site, and a β2-nt filler at the 3′-end. In the illustrated embodiment, the length of the 5′-filler is determined by the requirements of the restriction enzyme. The restriction site is used in cloning the intermediate fragment, and includes an ATG start codon. The 0-2-nt filler adjusts the reading frame of the intermediate fragment relative to the start codon. In some embodiments, the restriction site does not include a start codon. In some embodiments, a start codon is incorporated in the 3′-filler.

FIG. 5D is a schematic of the trailer (tlr) portion illustrated in FIG. 5B. From the 5′-end, the trailer comprises a 0-2-nt filler, a TAATAA stop sequence, a GGATCC restriction site, and a 5-nt filler. The 0-2-nt filler adjusts the reading frame of the stop codon relative to intermediate fragment. TAATAA is a pair of stop codons. Any suitable stop codon is useful. Some embodiments use one stop codon. GGATCC is a restriction site used for cloning the intermediate fragment. The length of the 3′-filler is determined by the requirements of the restriction enzyme. Those skilled in the art will understand that, in other embodiments, the leader and/or trailer use a different combination of fillers, restriction sites, start codon, and/or stop codons. In some embodiments, the intermediate fragment comprises a start and/or stop codon and the leader and/or trailer does not include the codon. For example, in the case of a synthetic gene, the gene typically includes both a start and stop codon. Similarly, those skilled in the art will understand that in some embodiments, the leader and/or trailer does not comprise a restriction site. Some embodiments of the leader and/or trailer do not use a 5′- and/or a 3′-filler.

A polypeptide expressed from a clone with an N−1 defect will be defective. The expressed peptide is analyzed using any means known in the art, for example, gel electrophoresis, capillary electrophoresis, two-dimensional electrophoresis, isoelectric focusing, spectroscopy, mass spectroscopy, NMR spectroscopy, chemically, ligand binding, enzymatic cleavage, or a functional or immunological assay. A clone that expresses the correct peptide is free from N−1 defects.

In some embodiments, the expressed polypeptide is analyzed using gel electrophoresis, which separates polypeptides by molecular weight. Of the 64 possible DNA codons, 3 are stop codons. Consequently, the frame-shift caused by a point deletion is likely to generate a new stop codon, resulting in a prematurely truncated polypeptide, the molecular weight of which is determined using gel electrophoresis. A clone that provides a full-length polypeptide is likely to have the desired sequence, while one that provides a truncated polypeptide is likely to have at least one point deletion. In some embodiments, a clone with an N−1 defect or defects produces a polypeptide that is too long, because the N−1 defect results in a frame-shift that causes the terminating stop codon(s) to be ignored (read through). In some embodiments, such a polypeptide that is too long will be terminated by a stop codon engineered into the expression vector downstream (3′) of the gene. As discussed above, some embodiments comprise three groups of stop codons, one group in each possible reading frame. In these embodiments, the molecular weight of the expressed polypeptide is higher than expected.

In some embodiments, analysis of the expressed polypeptide is used to narrow the sample of clones that are then sequenced. In these embodiments, the analysis of the expressed polypeptide is used to identify and to eliminate clearly defective (e.g., truncated or too long) DNA clones. The remaining clones are then sequenced. In these embodiments, the expression and analysis is a semi- or nonrandom selection method, in contrast to the random selection method described above. In some embodiments, the expressed polypeptide is analyzed by gel electrophoresis. In some cases, gel electrophoresis does not distinguish a defective polypeptide from the correct polypeptide. For example, in some cases a DNA sequence with an N−1 defect generates a defective polypeptide that, to within the resolution of the electrophoresis conditions, has the same molecular weight as the correct polypeptide. This scenario can arise where the defective DNA sequence fortuitously expresses a defective polypeptide similar in molecular weight to the correct polypeptide, for example, where the point defect is near the end of the clone. In another scenario, the clone has 3N point deletions that do not generate a new stop codon. As discussed above, the defective polypeptide is most likely shorter than the correct polypeptide. A defective polypeptide closer in molecular weight to the correct polypeptide than the resolution of the electrophoresis experiment is not distinguished. Given the resolving ability of gel electrophoresis, selecting a correct clone using the method is highly probable. The probability is further improved using an analytical technique with higher resolution, for example, capillary electrophoresis or mass spectroscopy. In some cases, all of the clones selected for sequencing in the gene expression screen have the correct sequence, indicating the reliability of this selection method. Furthermore, expressing a gene and determining the molecular weight of the expressed polypeptide is typically faster and/or less expensive than the equivalent amount of DNA sequencing. In some embodiments intermediate fragments are selected by estimating the molecular weight of the expressed polypeptide only, and DNA sequencing is reserved only for the final gene construct, and even then only after its molecular weight of a polypeptide expressed from the final gene has been estimated to be correct.

In some embodiments, all of the expressed polypeptides that are analyzed are defective, for example, truncated. In these embodiments, an analysis of the defective polypeptides indicates the location of the defect in the DNA sequence. The gene is then resynthesized using this information. In embodiments using multiple hierarchical synthesis steps, only some of the pieces of DNA are resynthesized, for example, an intermediate fragment containing the defect. In some embodiments, the offending fragment is divided in a different way and/or reoptimized, as discussed above. In some embodiments a different clone is chosen to replace the offending fragment.

In some embodiments, the DNA sequence is transformed into a selected organism such that a clone containing a correct and/or likely-to-be correct DNA sequence will exhibit a phenotype different from the phenotype exhibited by a clone containing an incorrect DNA sequence. The organism is a prokaryote or a eukaryote. Examples of suitable prokaryotes include bacteria, for example, E. coli. Examples of suitable eukaryotes include yeast, fungi, and mammalian cells. The differences in phenotype arise from mechanisms well known in the art, for example, differential induction and/or repression of gene expression by correct and incorrect DNA sequences, expression of different proteins or polypeptides by the correct and incorrect DNA sequences, and the like. In some embodiments, the difference in phenotype is detectable without specialized equipment, for example, by inspection by the naked eye. For example, in some embodiments, the difference in phenotype is the color of the organism. In other embodiments, the difference in phenotype is viability of the organism under particular conditions. Examples of particular conditions include pH, temperature, light, and the like. In some embodiments, conditions include the presence or absence of a particular compound or compounds, including, nutrients, for example, amino acids, carbohydrates, vitamins, cofactors and the like; and/or antibiotics or other toxic compounds. Those skilled in the art will understand that other particular conditions are compatible with the disclosed method. Those skilled in the art will understand that the embodiments described below are exemplary only, and that the method may be varied to use, for example, other organisms, phenotypes, conditions, genes, and vectors.

Some embodiments of the method use a vector referred to herein as a “frameshifted vector” to distinguish between correct and incorrect DNA sequences. The frameshifted vector is any type of vector known in the art useful for introducing a gene into an organism, for example, a plasmid, a cosmid, a phagemid, a bacteriophage, a virus, and/or a bacterium. The frameshifted vector contains a gene, that when expressed, changes the phenotype of a selected organism into which the vector is transformed, for example, color or viability. In the frameshifted vector, a frameshift is introduced into at least a portion of the open reading frame (ORF) of the gene such that the gene does not express a functional product. In some embodiments, the frameshift is introduced upstream of a functional portion of the gene. A functional portion of the gene is a portion that expresses a functional polypeptide or protein, the expression of which changes the phenotype of the organism. When an organism is transformed using the frameshifted vector, no functional product is expressed, and consequently, no change in phenotype is observed. The term “frameshift” as used herein is used in its usual sense, as well as to mean the insertion and/or deletion of one or more bases, which results in a change in reading frame. The three possible reading frames for the gene are referred to herein as the correct reading frame; the +1 or n+1 reading frame; and the −1 or n−1 reading frame. In a frameshifted vector, at least a portion of the ORF is in the −1 or +1 reading frame. Those skilled in the art will understand that for any particular vector/organism system, two frameshifted vectors, one with a −1 reading frame and one with a +1 reading frame are sufficient to perform the disclosed method with any synthetic DNA sequence designed to shift the downstream reading frame. In some embodiments, the frameshifted vector also includes at least one DNA insertion site upstream of the region of the gene that encodes the functional portion of the protein or peptide. The DNA insertion site is of any type known in the art useful for inserting a piece of DNA into the frameshifted vector. In some embodiments, the DNA insertion site is one or more restriction sites. Those skilled in the art will understand that any compatible restriction site known in the art is suitable. Examples of suitable restriction sites include, EcoR I, BamH I, Hind III, Pci I, Age I, Spe I, Nde I, Nco I, Sac I, Sac II, Pvu I, Xho I, Pst I, and Sph I. Some embodiments of the frameshifted vector comprise a plurality of DNA insertion sites.

The synthetic DNA sequence is designed to correct the frameshift when inserted into the DNA insertion site. In other words, the DNA sequence is designed with a length that corrects the −1 or +1 shift designed into the reading frame of the functional portion of the gene. A piece of DNA with the desired sequence is also referred to herein as having a “correct” DNA sequence. Consequently, the functional portion of the gene is in the correct reading frame when a correct DNA sequence is inserted therein. On transforming the selected organism with the resulting vector, the organism expresses functional polypeptide or protein, which changes the phenotype of the organism.

In contrast, when a DNA sequence with an N−1 defect is inserted into the DNA insertion site, the frameshift is not corrected. For example, for a vector with a −1 frameshift, inserting an N−1 DNA sequence produces a +1 frameshift. Inserting a DNA sequence with two N−1 defects also does not correct the frameshift. For a vector with a −1 frameshift, inserting a DNA sequence with two N−1 defects produces a −1 frameshift. Inserting a DNA sequence with three N−1 defects corrects the frameshift. In general, a DNA sequence with 3n N−1 defects will correct the frameshift, while those with 3n+1 or 3n+2 N−1 defects will not. Given the low error rates for incorporation of N−1 oligonucleotides in the synthesis of the next-larger piece of DNA provided above, especially for ligation-based methods, the probability that a DNA sequence will have three or more N−1 defects is low, although not negligible. Consequently, most of the DNA sequences with the correct reading frame in the frameshifted vector have the correct sequence. In some embodiments for selecting intermediate fragments, about 80% to about 95% of clones exhibiting the changed phenotype have the correct DNA sequence. The remainder have three or more N−1 defects, which is consistent with the error rates provided above.

Similarly, when an organism is transformed with a frameshifted vector into which no DNA is inserted, the frameshift engineered into the vector causes no functional product to be expressed, and consequently, no change in phenotype. Of the four most likely DNA inserts into the frameshifted vector—no DNA, DNA with one N−1 defect, DNA with two N−1 defects, and correct DNA—only the vector with the correct DNA inserted therein changes the phenotype of an organism transformed therewith. An organism exhibiting the changed phenotype is selected, the correct DNA sequence isolated, and the DNA sequence used as described herein. The correct DNA sequence is isolated by any method known in the art, for example, by excision or by PCR using suitable primers.

A frameshifted vector is synthesized by any method known in the art. Some embodiments use known combinations of a particular vector and organism such that the organism changes phenotype when transformed with the vector. Typically, the vector has an open reading frame containing a functional portion of a gene, the expression of which changes the phenotype of the organism. One or more bases are inserted and/or deleted in the ORF upstream of the functional portion of the gene, thereby causing a −1 or +1 frameshift in the functional portion of the gene. The bases are inserted and/or deleted by methods known in the art, for example, cutting with restriction enzymes, digestion of double- or single-stranded portions, site-directed mutagenesis, ligation, chemical synthesis, and the like. In some embodiments, a DNA insertion site is also engineered upstream of the functional portion of the gene. In other embodiments, the ORF contains a preexisting DNA insertion site upstream of the functional portion of the gene.

Some embodiments distinguish a correct DNA sequence from an incorrect DNA sequence by the color of the transformed organism. An embodiment of the method uses a vector with a gene encoding the α-complementing fragment (α-fragment) of E. coli lacZ β-galactosidase. The DNA sequence is inserted at the DNA insertion site located upstream of the functional portion of the gene for the α-complementing fragment. The vector is engineered so that functionality of the α-fragment gene depends on the reading frame of the functional portion of the gene after the synthetic DNA sequence is inserted into the DNA insertion site. The vector is transformed into an E. coli strain containing a 5′-truncation of the lacZ gene. Protein expressed by a functional α-fragment gene transcomplements the defective lacZ expressed by the cell, thereby producing functional β-galactosidase. When the cells are grown on indicator media containing isopropylthio-β-D-galactoside (IPTG) and 5-bromo-4-chloro-3-indolyl-β-D-galactoside (X-Gal), colonies developing from cells with a functional α-complementing fragment gene are blue, while those with a defective α-complementing fragment gene are white.

In one embodiment, the frameshifted vector is a modified pGEM®-3Z vector (Promega Corp., Madison, Wis.). The pGEM®-3Z vector is a pBR322-based plasmid that contains a multiple cloning site (MCS) in the ORF of the α-fragment gene, as well an ampicillin resistance gene. The vector is engineered with a frameshift mutation in the α-fragment gene, which renders the gene non-functional. The DNA sequence is designed to correct the frameshift when inserted at the MCS, thereby producing a functional α-fragment gene. Colonies of cells transformed with the frameshifted vector are white. White colonies are also observed for cells transformed with a frameshifted vector into which a DNA sequence with one or two N−1 defects is inserted. Blue colonies are observed only for cells transformed with a frameshifted vector into which a DNA sequence with no defects is inserted. A feature of this system is that the α-fragment is known to retain its activity with up to 650 amino acid extensions at the N-terminus.

Is some embodiments, the difference in phenotype is temperature resistance. Some embodiments use E. coli AB4141 (F⁻, metC56, lct-1, thi-1, valS7, ara-14, lacY1, galK2, xyl-7, rpsL69, tfr-5, supE44), which contains a conditionally lethal, temperature sensitive valS (valyl-tRNA synthetase). This strain grows at a permissive temperature of about 37° C., but not at a restrictive temperature of about 42° C. After transformation with a plasmid expressing wild-type valS, the strain grows at the restrictive temperature. One embodiment uses a frameshifted vector derived from the plasmid pDH-1Δ11, which includes a wild-type valS gene. In this system, any colony growing at the restrictive temperature has the correct DNA sequence. Cells without the correct DNA sequence do not grow at all at the restrictive temperature.

Some embodiments provide a kit comprising one or more frameshifted vectors and instructions for using the frameshifted vector(s) to isolate a DNA sequence as described herein. Some embodiments of the kit also include other components, for example, a preselected organism, a growth medium, a restriction enzyme, and the like.

The method described herein provides a quick, easy, and inexpensive method for constructing a synthetic DNA gene that encodes any desired protein or any other desired nucleic acid. A first example provided herein describes a two-step recursive assembly of a gene encoding E. coli threonine deaminase, a protein with 514 amino acid residues (1,542 coding bases). A second example describes a three-step recursive assembly of a gene encoding the smallpox (variola) DNA polymerase, a protein with 1,005 amino acid residues (3,015 coding bases). A third example describes a direct self-assembly of a synthetic E. coli threonine deaminase gene, with reassembly by cloning into an expression vector. A fourth example describes a two-step recursive assembly with sampling and sequencing of the 876 bp Ty3 GAG3 gene, which encodes the Gag3p polyprotein. Ty3 is a retrotransposon in Saccharomyces cerevisiae. A fifth example describes a two-step recursive assembly with sampling and sequencing for the 1640 bp Ty3 integrase (“Ty3 IN”) gene. Accordingly, it will be appreciated that the method described herein may be used to construct any desired nucleic acid sequence or any desired gene.

E. coli threonine deaminase was chosen arbitrarily because (1) its size is comparable to most proteins, demonstrating wide applicability, (2) it assembles into a homo-tetramer, demonstrating protein-protein interactions, (3) its allosteric properties easily can be assessed for correct folding and assembly, and (4) in part by whim for old times sake because its structure and properties was the Ph.D. thesis project of one of us. See, Hatfield & Ray “Coupling of slow processes to steady state reactions” J. Biol. Chem., 1970, 245(7), 1753-4; Hatfield, Ray, & Umbarger “Threonine deaminase from Bacillus subtilis. III. Pre-steady state kinetic properties.” J. Biol. Chem., 1970, 245(7), 1748-53; Hatfield & Umbarger “Threonine deaminase from Bacillus subtilis. II. The steady state kinetic properties.” J. Biol. Chem., 1970, 245(7), 1742-7; Hatfield & Umbarger “Threonine deaminase from Bacillus subtilis. I. Purification of the enzyme.” J. Biol. Chem., 1970, 245(7), 1736-41.

Smallpox (variola) DNA polymerase was chosen because (1) it is larger than most proteins, demonstrating wide applicability, and (2) current events render it of special interest. In particular, the ability to synthesize de novo a gene from a pathogenic organism permits study without actual use of the pathogen. Examples of such studies include regulation of gene expression, drug development, vaccine development, and the like. Because the pathogen is never used, there is no chance of exposure, either accidental or intentional. Moreover, the sequence of the synthetic gene may be modified to optimize expression in the selected, non-pathogenic organism.

Ty3 was chosen because of its resemblance to retroviruses. GAG3 encodes Gag3p, a 38 kDa polyprotein that is processed into a 26 kDa capsid and 9 kDa nucleocapsid that assemble into virus-like particles (VLP). Ty3 IN is implicated in the retrovirus-like integration of Ty3 in the S. cerevisiae genome.

Preferred embodiments of the disclosed method are illustrated in the following Examples. In these Examples, the terms “Medium-sized piece” and “Intermediate Fragment” are used interchangeably.

### EXAMPLE 1

### E. coli Threonine Deaminase by Two-Step Recursive Decomposition and Overlap Extension Assembly

EXAMPLE 1 illustrates the synthesis of an E. coli threonine deaminase gene by a two-step hierarchical decomposition and reassembly by overlap extension. E. coli threonine deaminase is a protein with 514 amino acid residues (1,542 coding bases).

Design

The sequence design method permuted synonymous (silent) codon assignments to each amino acid in the desired protein sequence. Each synonymous codon change results in a different artificial gene sequence that encodes the same protein. Because E. coli was the desired expression vector, the initial codon assignment was to pair each amino acid with its most frequent codon according to E. coli genomic codon usage statistics. Subsequently, the codon assignments were perturbed as described below. The final codon assignment implied a final DNA sequence to be achieved biochemically.

In this two-step hierarchical decomposition, the gene was divided first into five overlapping medium-sized pieces (in the present example, not longer than 340 bases, overlap not shorter than 33 bases), then each medium-sized piece was divided into several overlapping short segments (in the present example not longer than 50 bases, overlap not shorter than 18 bases). All overlaps were lengthened if necessary to include a terminal C or G for priming efficiency.

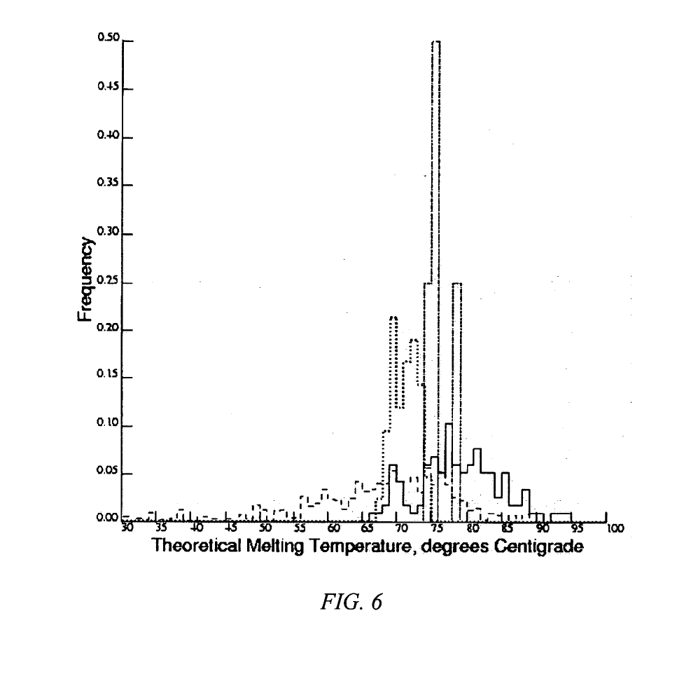

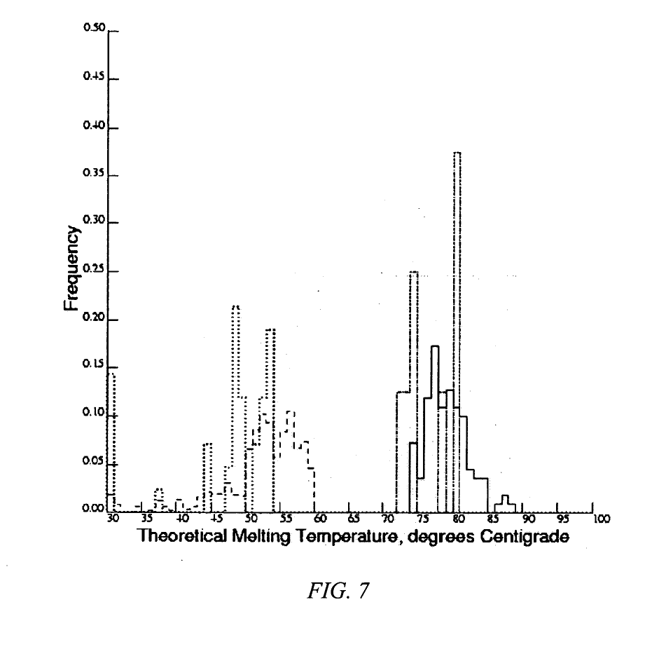

Theoretical melting temperatures were calculated with Mfold for all possible correct and incorrect hybridizations of the medium-sized and short pieces of DNA using the most common codons. The results are illustrated in FIG. 6. The gap between the lowest-melting correct match and the highest-melting incorrect match was increased by perturbing the codon assignments as described above. Theoretical melting temperatures for the optimized sequences are illustrated in FIG. 7. In the present example, the gap was at least 10° C.

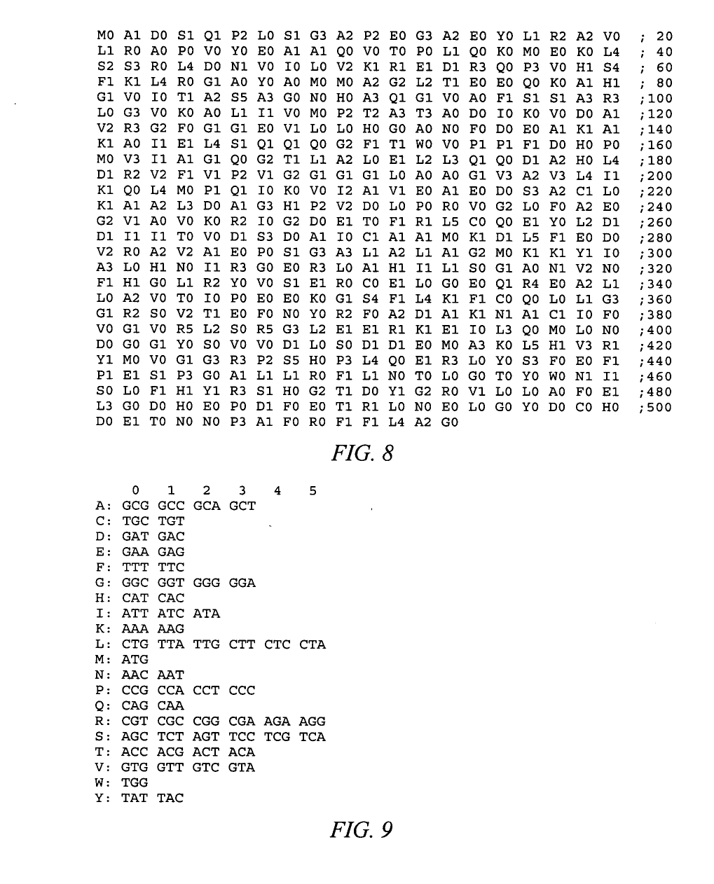

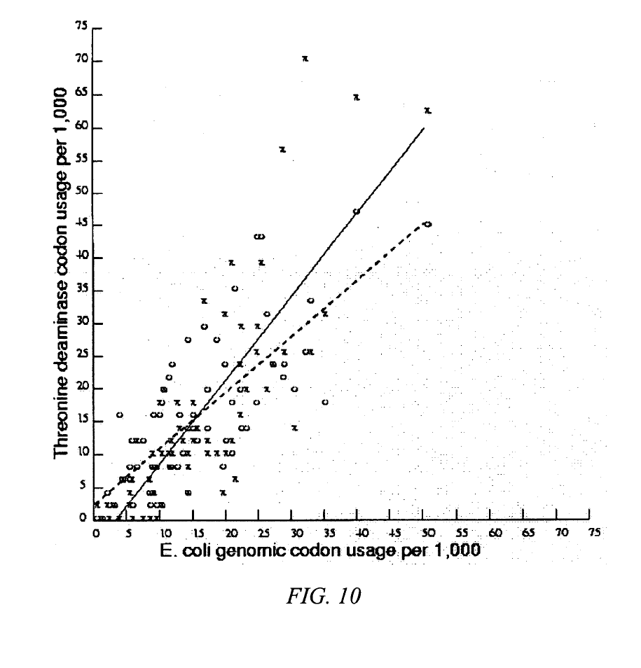

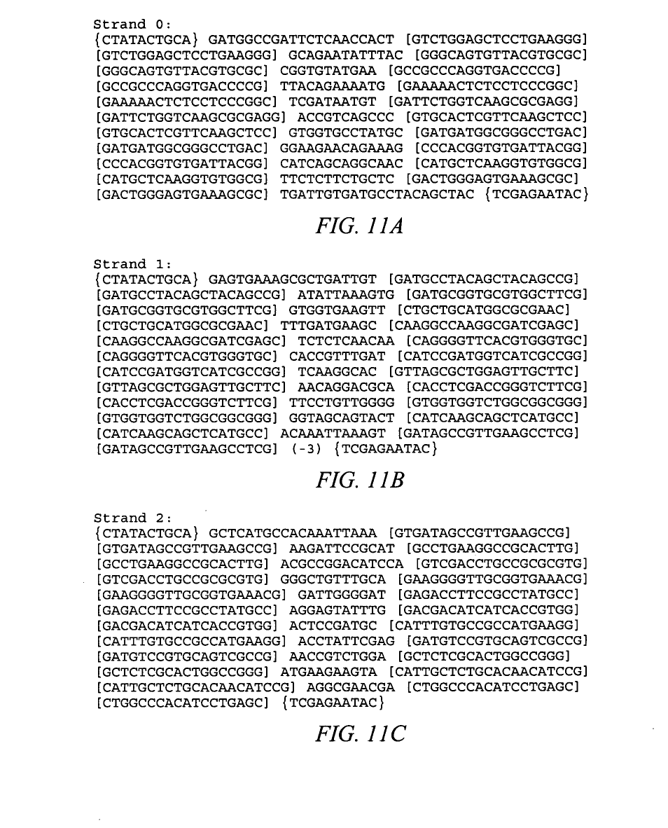

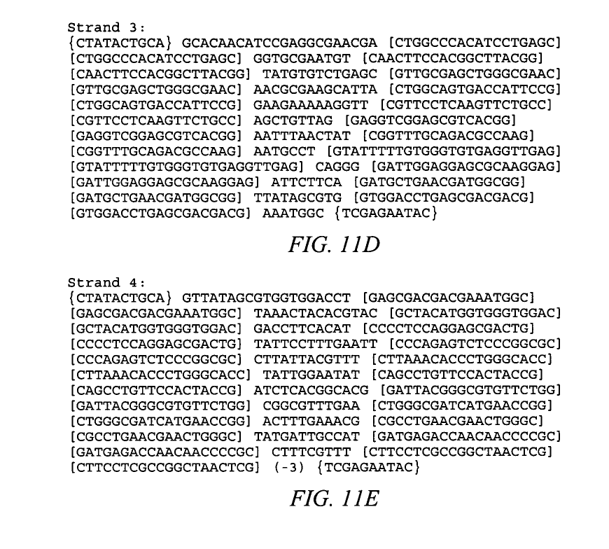

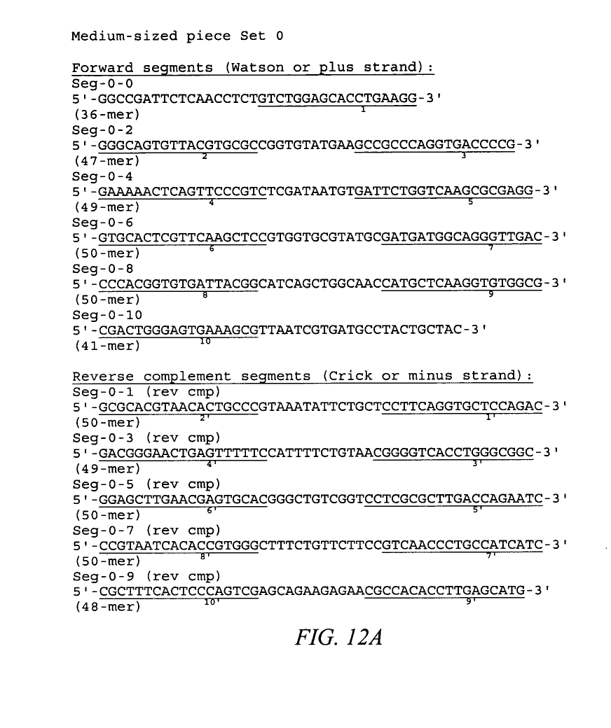

The final codon assignment to every amino acid in the threonine deaminase protein sequence is provided in FIG. 8 (SEQ. ID. NO.: 1). FIG. 9 is the codon assignment key for E. coli. FIG. 8 forms the basis for FIG. 7, FIG. 10, and FIG. 11A-FIG. 1D. The initial codon assignment (column 0 in FIG. 9) forms the basis for FIG. 6.

The design objectives may be achieved without excessive use of rare codons as illustrated in FIG. 10. The codon usage in the present example (open circles and dashed line) is shown as a function of genomic codon usage in E. coli. The correlation coefficient of codon usage here with codon usage in E. coli is 0.76. For comparison, the native codon usage in natural threonine deaminase is also shown (x's and solid line). The correlation coefficient of native codon usage with codon usage in E. coli is 0.81. The native usage is better than, but comparable to, the designed.

The resulting DNA sequence was decomposed into the short overlapping segments shown in the overlap maps illustrated in FIG. 11A-FIG. 11E. The overlaps between short segments are indicated by brackets: [ ]. The overlaps between long strands are indicated by braces: { }. In the depiction of strands 1 and 4, three bases preceding the (−3) are the same bases as the first three bases shown after the (−3) in order to clearly illustrate the overlaps. These bases are not repeated in the actual DNA sequences. SEQ. ID. NO.: 2-SEQ. ID. NO.: 12 correspond to the sequences that comprise strand 0 (FIG. 11A), SEQ. ID. NO.: 13-SEQ. ID. NO.: 24 correspond to the sequences that comprise strand 1 (FIG. 11B), SEQ. ID. NO.: 25-SEQ. ID. NO.: 36 correspond to the sequences that comprise strand 2 (FIG. 11C), SEQ. ID. NO.: 37-SEQ. ID. NO.: 48 correspond to the sequences that comprise strand 3 (FIG. 1D), and SEQ. ID. NO.: 49-SEQ. ID. NO.: 60 correspond to the sequences that comprise strand 4 (FIG. 11E). As is described in greater detail below, each short segment was synthesized directly and assembled into the five medium-sized pieces of DNA in five parallel reactions. In a second stage, the five medium-sized pieces were assembled into the synthetic gene. The segments shown in FIG. 11A-FIG. 11E plus the various primers for overlap extension totaled 72 synthesized segments with a total of 3,093 nucleotides.

Synthesis

The target DNA sequence of the synthetic E. coli L-threonine deaminase gene has 1,542 bases. In the present example, the target DNA sequence was decomposed into a set of five medium-sized pieces, each medium-sized piece overlapping the adjacent medium-sized piece by at least 33 bases. In turn, each medium-sized piece was decomposed into “sets” of 11 or 12 small, single-stranded DNA segments, which overlapped the adjacent segment by from 18 to 50 bases. The five medium-sized pieces are designated “Medium-sized piece 0” through “Medium-sized piece 4” herein. The small single-stranded DNA segments that make up the medium-sized pieces are designated as “Seg,” medium-sized piece number, and segment number, where the segment number starts at 0 starting at the 5′-end of the forward segment. Note that the numbering for both the medium-sized pieces and the segments begins at zero. For example, the first segment of Medium-sized piece 0 is “Seg-0-0”; the seventh segment of Medium-sized piece 4 is “Seg-4-6.” The segments and primers were commercially synthesized by Illumina, Inc., San Diego, Calif.

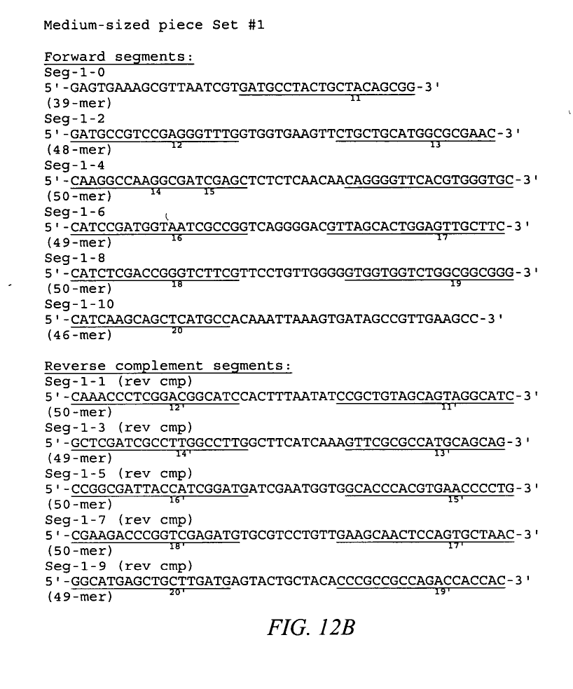

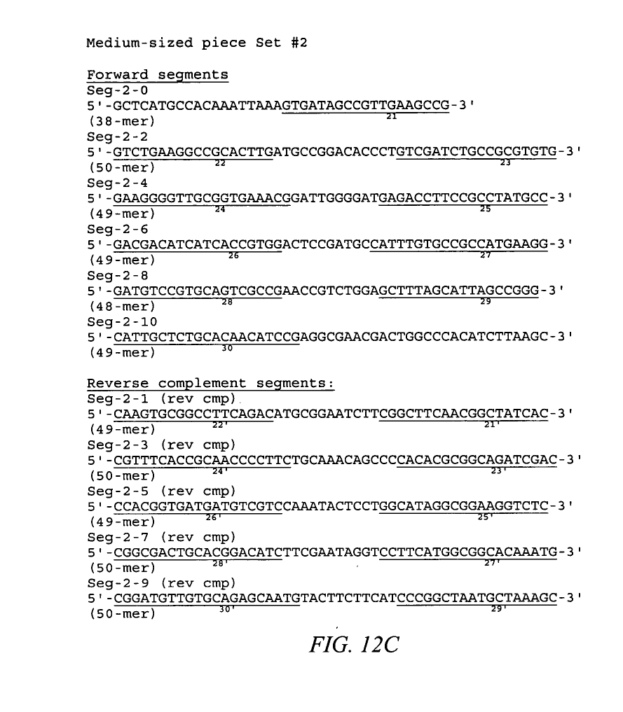

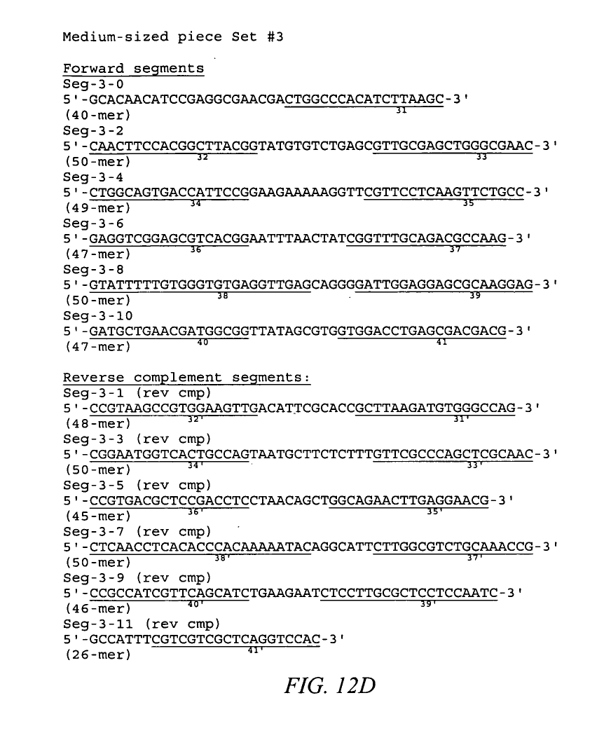

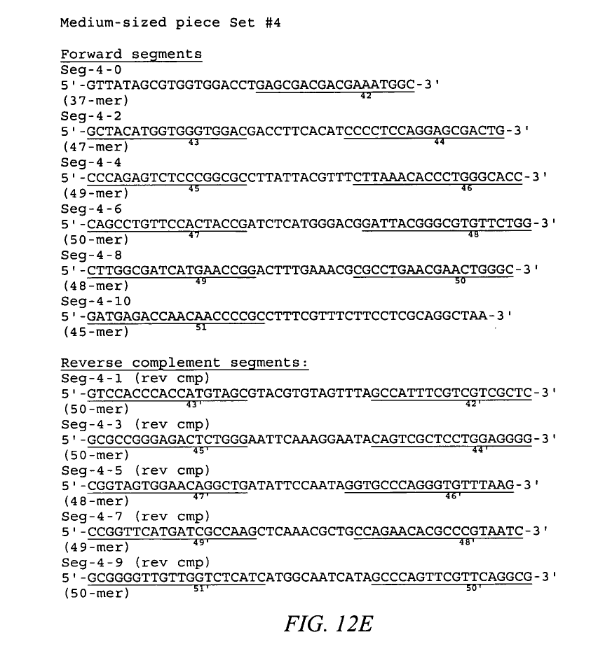

FIG. 12A-FIG. 12E illustrate the single-stranded DNA segments used to construct Medium-sized piece 0 through Medium-sized piece 4, respectively. For each segment, the region that overlaps the adjacent complementary strand is underlined and numbered. Overlaps identified by primed numbers are complementary to the corresponding unprimed overlaps. For example, sequence 1 is complementary to sequence 1′ of the adjacent complementary strand, while sequence 2 is complementary to sequence 2′, and so forth. SEQ. ID. NO.: 61-SEQ. ID. NO.: 71 correspond to Seg-0-0, Seg-0-1, Seg-0-2 . . . Seg-0-10 illustrated in FIG. 12A, respectively. Similarly, SEQ. ID. NO.: 72-SEQ. ID. NO.: 82 correspond to Seg-1-0 through Seg-1-10 (FIG. 12B), SEQ. ID. NO.: 83-SEQ. ID. NO.: 93 correspond to Seg-2-0 through Seg-2-10 (FIG. 12C), SEQ. ID. NO.: 94-SEQ. ID. NO.: 105 correspond to Seg-3-0 through Seg-3-11 (FIG. 12D), and SEQ. ID. NO.: 106-SEQ. ID. NO.: 116 correspond to Seg-4-0 through Seg-4-10 (FIG. 12E).

Leader and Trailer Primers

For Medium-sized piece 0, the first segment, Seg-0-0, serves as the leader primer for overlap extension. The trailer primer (reverse complement) is 5′-GTAGCAGTAGGCATCAC-3′ (17-mer, SEQ. ID. NO.: 117). For Medium-sized piece 1, segment Seg-1-0 serves as the leader primer and the trailer primer is 5′-GGCTTCAACGGCTATCAC-3′ (18-mer, SEQ. ID. NO.: 118). For Medium-sized piece 2, segment Seg-2-0 serves as the leader primer and the trailer primer is 5′-GCTTAAGATGTGGGCCAG-3′ (18-mer, SEQ. ID. NO.: 119). For medium-sized piece 3, Seg-3-0 serves as the leader primer and Seg-3-11 serves as the trailer primer. For Medium-sized piece 4, segment Seg-4-0 serves as the leader primer and the trailer primer is 5′-TTAGCCTGCGAGGAAGAAAC-3′ (20-mer, SEQ. ID. NO.: 120). Those skilled in the art will appreciate that the segments may be designed such that no added primers are used, as for Medium-sized piece 3, such that one added primer is used, as for Medium-sized pieces 0-2, and 4, or such that two added primers are used, not illustrated. In particular, if an even number of segments is used, the segments may be designed such that no added primers are needed where the first segment serves as the leader primer and the last segment serves as the trailer primer. It will also be appreciated that different flanking sequences may be added easily to these leaders and trailers.

Assembly of the Five Medium-sized Pieces

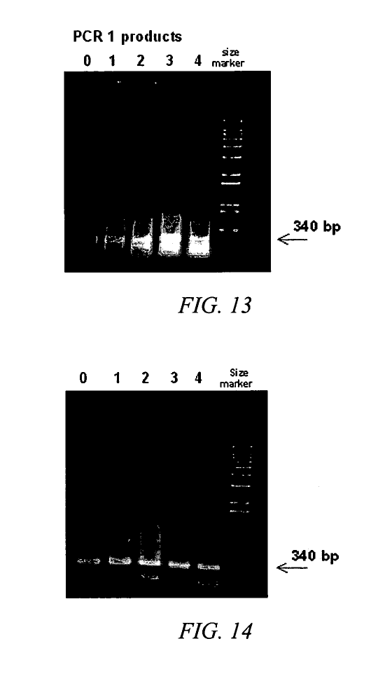

First Overlap Extension Reactions. The five Medium-sized pieces were constructed in parallel by overlap extension and PCR from the appropriate set of single-stranded DNA sequences. The reaction mixture is provided in TABLE III and the thermocycler conditions in TABLE IV. The products of the overlap extension reactions were separated on a 1% agarose gel, shown in FIG. 13.

TABLE III

| Reagent | Quantity |
|---|---|
| Roche High Fidelity PCR Master, Vial #1 | 25.0 μL |
| (Product # 2140314) | |
| Synthetic oligonucleotide mix* | 1.0 μL |
| Leader primer, 50 μM | 0.5 μL |
| Trailer primer, 50 μM | 0.5 μL |
| Sterile water, Roche (molecular biology grade) | 23.0 μL |

*For each of the synthetic oligonucleotide sets (0-4), the forward and reverse complement synthetic oligonucleotides were mixed in equal amounts to a final concentration of 27.5 ng/μL.

TABLE IV

| Step | Conditions |
|---|---|
| 1. | 94° C. for 5 minutes for initial denaturation, |
| 2. 25 cycles of: | 94° C. for 1 minute |
| | 45° C. for 30 seconds |
| | 65° C. for 5 minutes |
| 3. | 65° C. for 10 minutes for final extension |

PCR Reaction (enrichment). Each Medium-sized piece was separately enriched by PCR using the reaction mixture provided in TABLE V and the thermocycler conditions provided in TABLE VI. The products of the PCR reactions were separated on a 1% agarose gel shown in FIG. 14.

TABLE V

| Reagent | Quantity |
|---|---|
| Roche High Fidelity PCR Master, Vial #1 | 25.0 μL |
| (Product # 2140314) | |
| 1:50 dilution of first overlap extension reaction | 1.0 μL |
| product | |
| Leader primer, 50 μM | 0.5 μL |
| Trailer primer, 50 μM | 0.5 μL |
| Sterile water, Roche (molecular biology grade) | 23.0 μL |

TABLE VI

| Step | Conditions |
|---|---|
| 1. | 94° C. for 5 minutes for initial denaturation, |
| 2. 25 cycles of: | 94° C. for 1 minute |
| | 45° C. for 30 seconds |
| | 65° C. for 5 minutes |
| 3. | 65° C. for 10 minutes for final extension |

Assembly of the Five Medium-Sized Pieces into a Full Length Gene

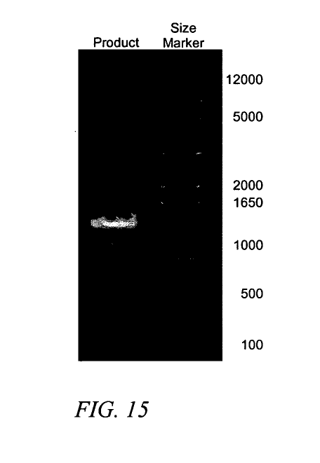

Second Overlap Extension Reaction. The synthetic threonine deaminase gene was constructed from the five medium-sized pieces from the PCR reactions using the reaction mixture provided in TABLE VII and the thermocycler conditions provided in TABLE VIII. After the reaction was complete, product was run on a 1.2% agarose gel (FIG. 15) and the band corresponding to the threonine deaminase gene was purified from the gel with GENECLEAN® (BIO101 Systems®, Qbiogene). The purified gene was further purified with phenol-chloroform (1:1) and chloroform-isoamyl alcohol (24:1). The aqueous layer was ethanol precipitated with glycogen, and the precipitate resuspended in water and digested with Nde I and BamH I. Those skilled in the art will appreciate that other restriction sites could be incorporated in the synthetic gene.

TABLE VII

| | |
|---|---|
| Reagent | Quantity |

| | |
|---|---|
| PCR Reaction Product, | 2.0 μL |
| Medium-sized piece 0 | |
| PCR Reaction Product, | 2.0 μL |
| Medium-sized piece 1 | |
| PCR Reaction Product, | 2.0 μL |
| Medium-sized piece 2 | |
| PCR Reaction Product, | 2.0 μL |
| Medium-sized piece 3 | |
| PCR Reaction Product, | 2.0 μL |
| Medium-sized piece 4 | |
| Leader primer, 50 μM¹ | 1.0 μL |
| Trailer primer, 50 μM² | 1.0 μL |
| Sterile water, Roche | 13.0 μL |
| (molecular biology grade) | |
| Roche High Fidelity PCR Master, | 25.0 μL |
| Vial #1 (Product # 2140314) | |

¹Forty-five base pair leader primer with Nde I endonuclease restriction enzyme site (bold): 5′-CTATATCTAGCATATGGCCGATTCTCAACCTCTGTCTGGAGCACC-3′ (SEQ. ID. NO.: 121).
²Forty-five base pair trailer primer (reverse complement) with BamH I endonuclease restriction enzyme site (bold): 5′-GTATTGGATCCTTAGCCTGCGAGGAAGAAACGAAAGGCGGGGTTG-3′ (SEQ. ID. NO.: 122).

TABLE VIII

| Step | Conditions |
|---|---|
| 1. | 94° C. for 5 minutes for initial denaturation, |
| 2. 20 cycles of: | 94° C. for 1 minute |
| | 60° C. for 30 seconds |
| | 65° C. for 10 minutes |
| 3. | 65° C. for 10 minutes for final extension |

Ligation and Expression

Directional cloning is performed on the purified threonine deaminase gene. pET14b Expression vector (Novagen) is cleaved with BamH I and Nde I generating compatible termini to the threonine deaminase. The threonine deaminase insert is ligated into the vector and this is used to transform into BL21 DE3 electro competent cells.

### EXAMPLE 2

### variola DNA Polymerase by Three-Step Recursive Decomposition and Overlap Extension Assembly

EXAMPLE 2 illustrates the synthesis of a variola DNA polymerase gene by a three-step hierarchical decomposition and reassembly by overlap extension. variola DNA polymerase is a protein with 1,005 amino acid residues (3,015 coding bases). variola DNA polymerase is also referred to as “Varpol” and “vpol” herein. Design

Because the variola DNA polymerase gene was intended for expression in E. coli, codon selection considerations were similar to those used in the design of E. coli threonine deaminase described in EXAMPLE 1.

The three-step hierarchical decomposition was performed as follows. First, the gene was divided into two large pieces of about 1,500 bases. The first large piece is designated herein as “Part I” or “polymerase-1.” The second large piece is referred to herein as “Part II” or “polymerase-2.” Parts I and II were designed with complementary Apa I sites to allow their reassembly by ligation. Second, each large piece was divided into five overlapping medium-sized pieces (Intermediate Fragments), in the present example, not longer than 340 bases, with overlaps not shorter than 33 bases. Third, each medium-sized piece was divided into several overlapping short segments, in the present example not longer than 50 bases, with overlaps not shorter than 18 bases. All overlaps were lengthened if necessary to include a terminal C or G for DNA polymerase priming efficiency.

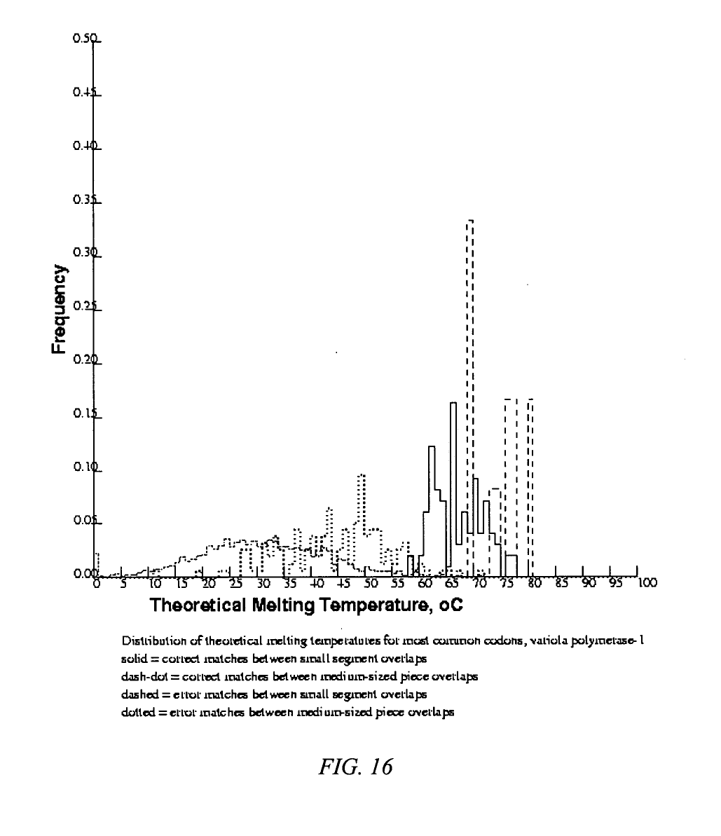

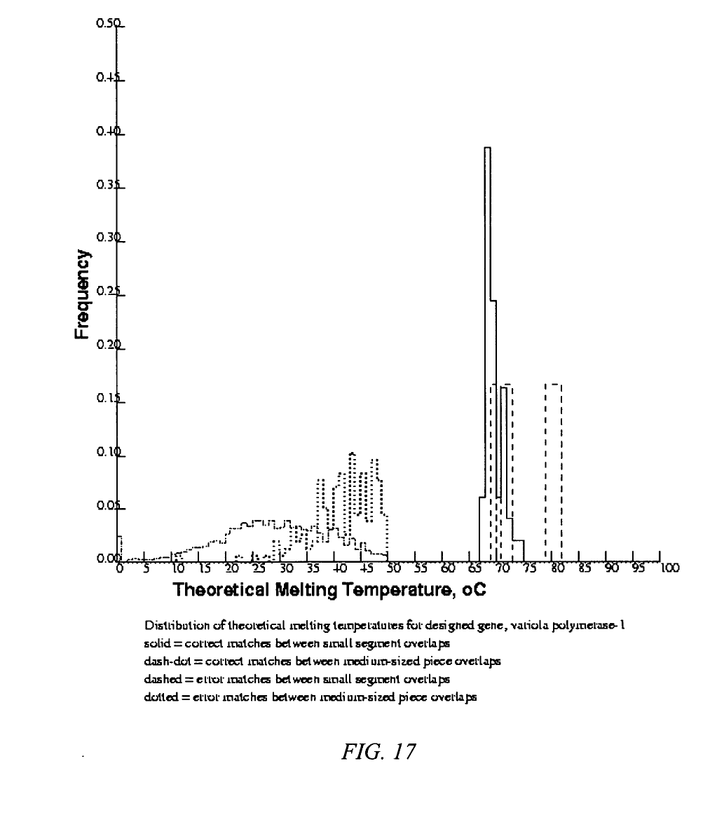

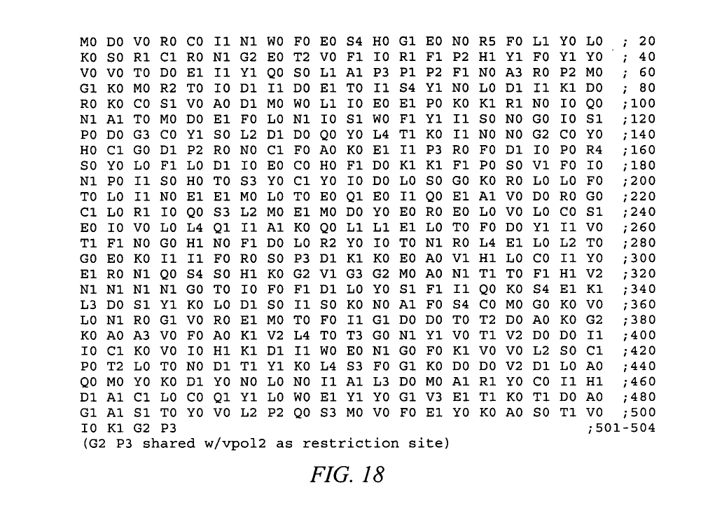

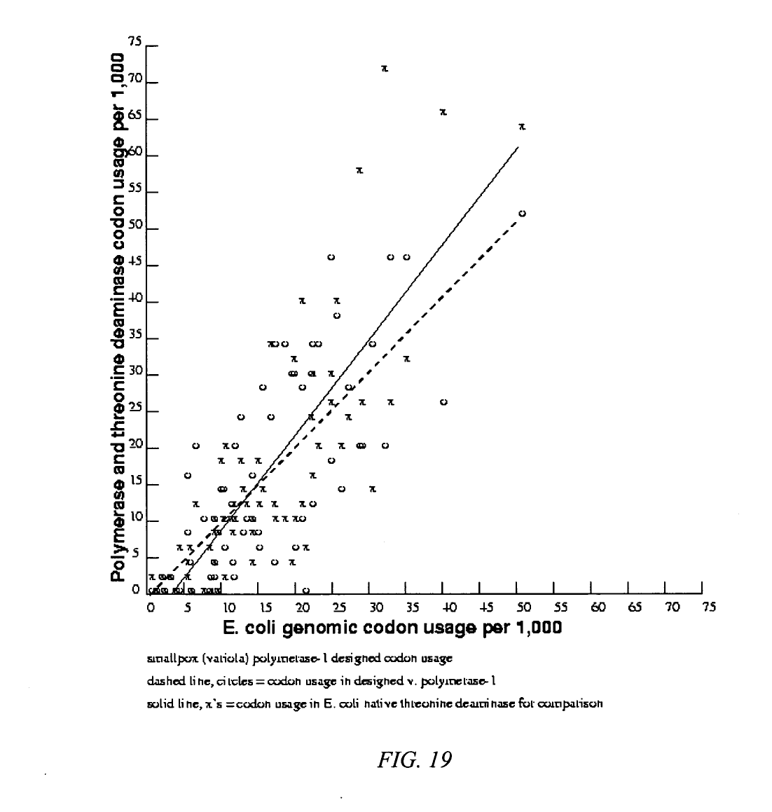

The sequences of the DNA pieces were perturbed to provide a suitable gap between the lowest-melting correct match and the highest-melting wrong match. Theoretical melting temperatures were calculated as described in EXAMPLE 1. For Part I of the gene, the melting temperatures of the correct and incorrect matches for the small and medium-sized pieces for the most common E. coli codons are provided in FIG. 16 and for the designed sequence in FIG. 17. The designed DNA sequence is provided in FIG. 18 (SEQ. ID. NO.: 123), which is based on the codon key in FIG. 9 of EXAMPLE 1. A comparison of the codon usage in the designed Part I sequence compared to the codon usage in native E. coli threonine deaminase is provided in FIG. 19, illustrating the similarity in codon frequency.

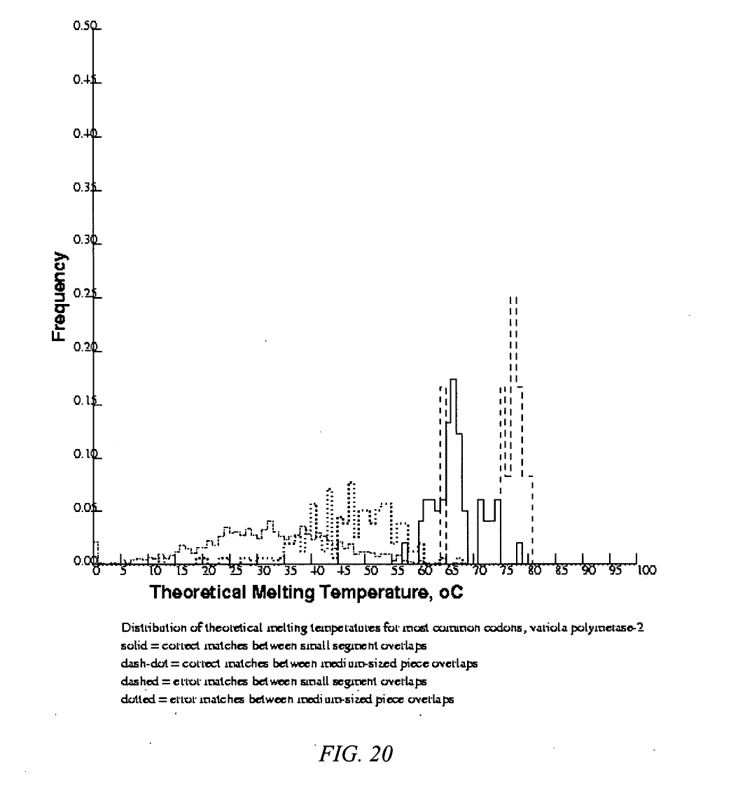

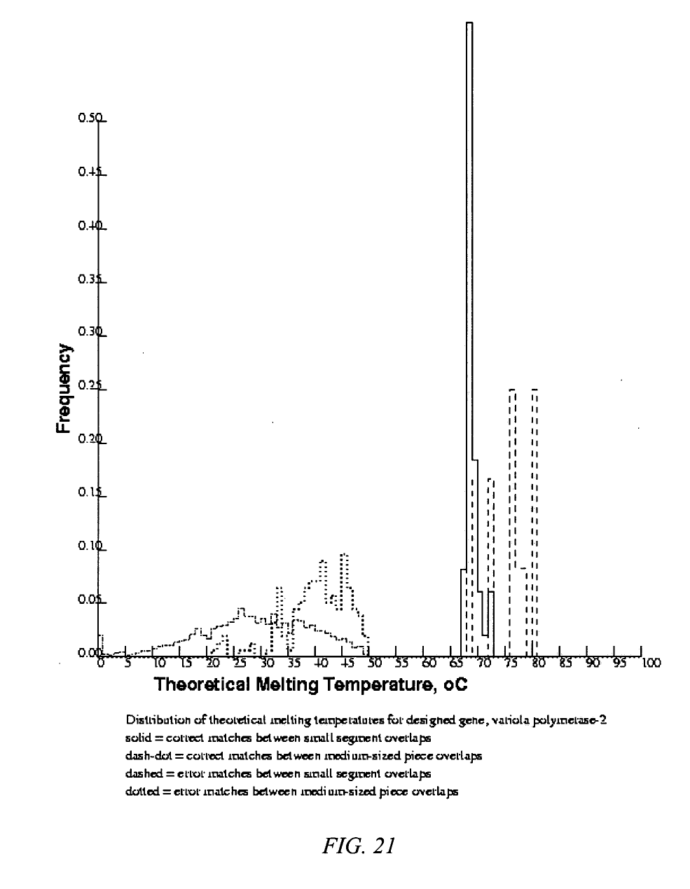

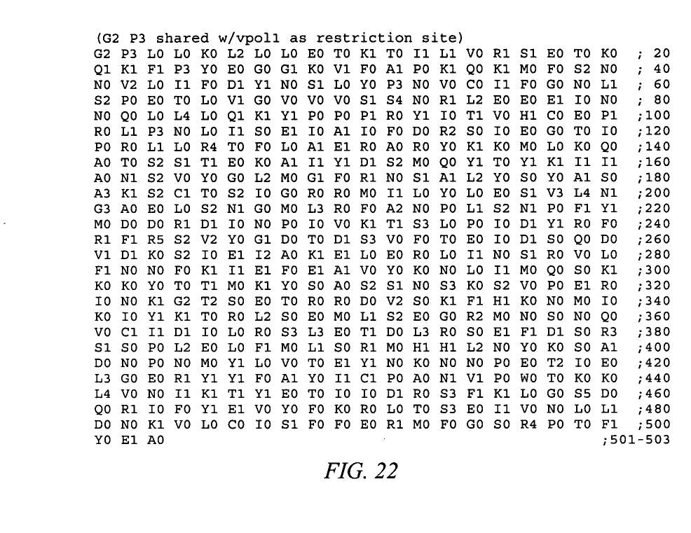

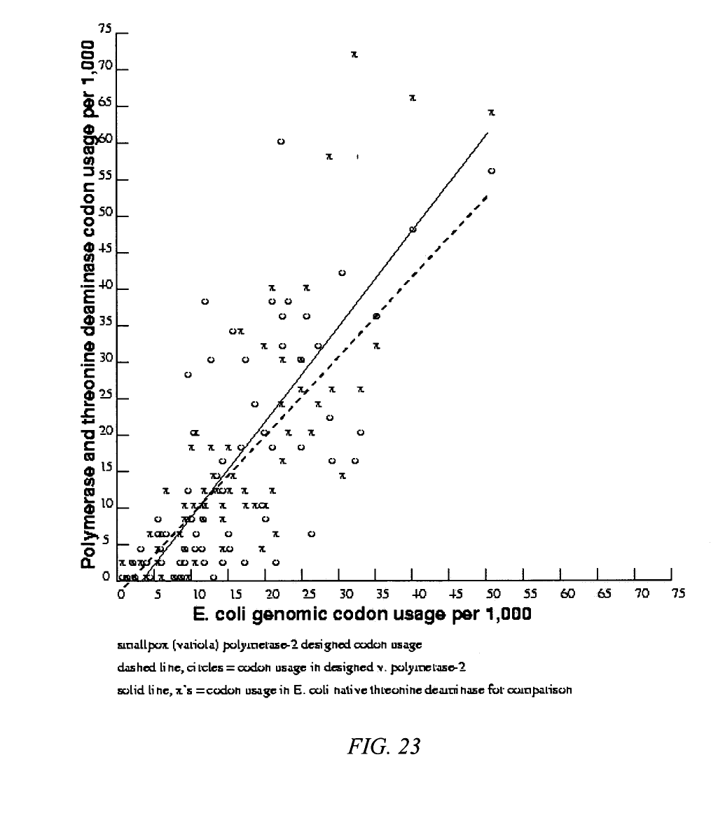

Theoretical melting temperatures for the small and medium-sized pieces of Part II of the variola DNA polymerase gene are provided in FIG. 20 for the most common codons of E. coli, and for the designed sequences in FIG. 21. The designed sequence for Part II is provided in FIG. 22 (SEQ. ID. NO.: 124), which is based on the codon key in FIG. 9 of EXAMPLE 1. A comparison of the codon usage of native E. coli threonine deaminase with the designed sequence of Part II is provided in FIG. 23, illustrating the similarity in codon frequency.

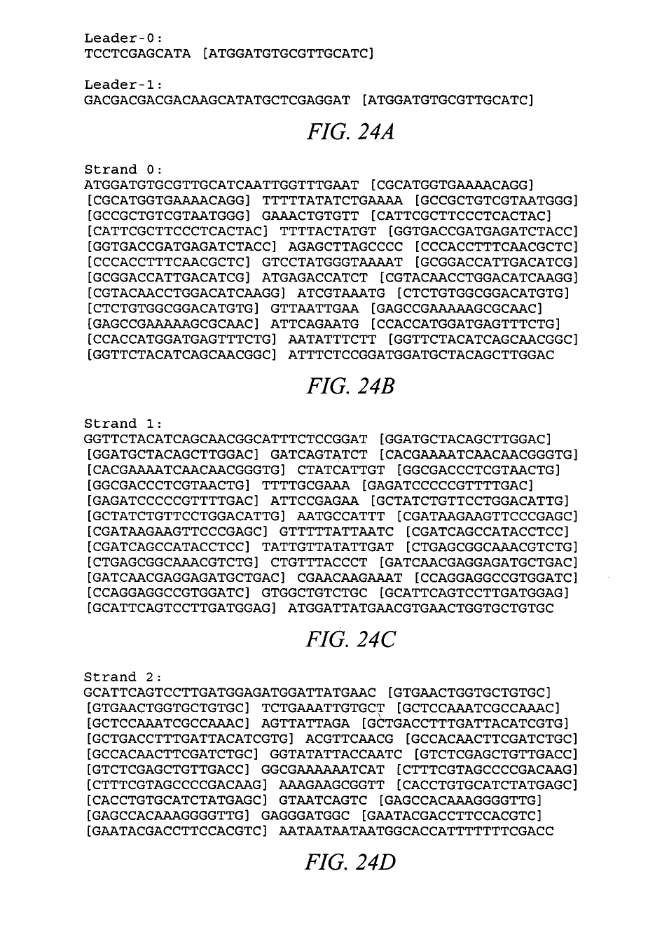

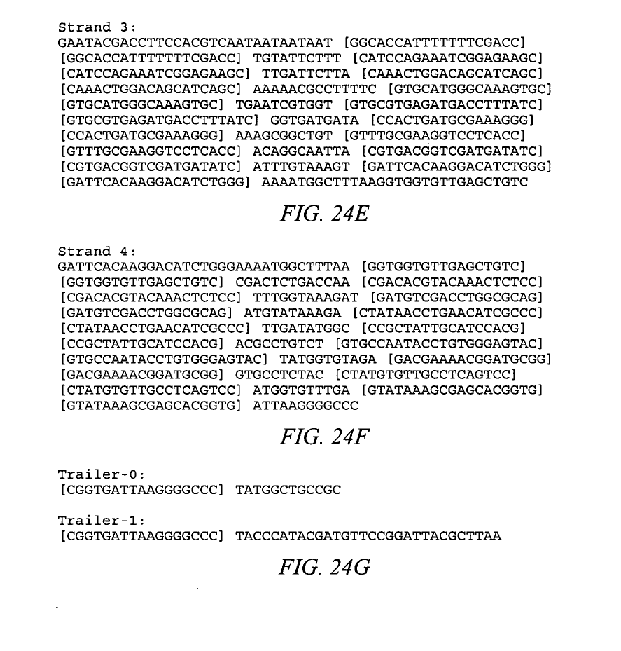

Overlap maps for the overlapping short segments used to build each of the five Intermediate Fragments (medium-sized pieces) 0-4, leaders, and trailers that make up Part I of the variola DNA polymerase gene are provided in FIG. 24A-FIG. 24G. SEQ. ID. NO.: 125 corresponds to leader-0 (FIG. 24A), SEQ. ID. NO.: 126 corresponds to leader-1 (FIG. 24A), SEQ. ID. NO.: 127-SEQ. ID. NO.: 138 correspond to strand-0 (FIG. 24B), SEQ. ID. NO.: 139-SEQ. ID. NO.: 150 correspond to strand-1 (FIG. 24C), SEQ. ID. NO.: 151-SEQ. ID. NO.: 160 correspond to strand-2 (FIG. 24D), SEQ. ID. NO.: 161-SEQ. ID. NO.: 170 correspond to strand-3 (FIG. 24E), SEQ. ID. NO.: 171-SEQ. ID. NO.: 180 correspond to strand-4 (FIG. 24F), SEQ. ID. NO.: 181 corresponds to trailer-0 (FIG. 24G), SEQ. ID. NO.: 182 corresponds to trailer-1 (FIG. 24G). As is described in greater detail below, each short segment shown in FIG. 24A-FIG. 24G was synthesized directly and assembled into the five Intermediate Fragments in five parallel reactions, then in a second stage, the five Intermediate Fragments were assembled into Part I of the variola DNA polymerase gene.

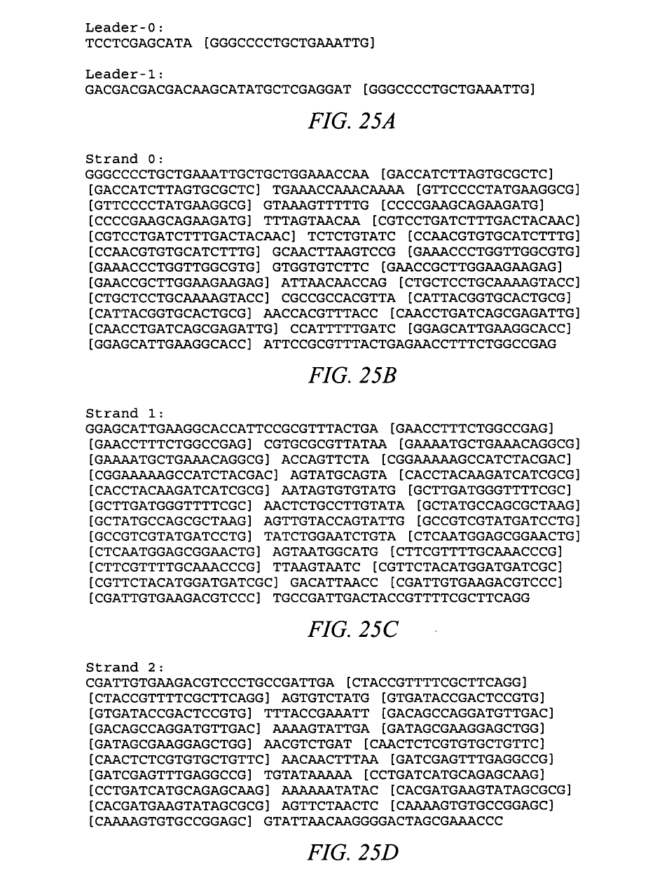

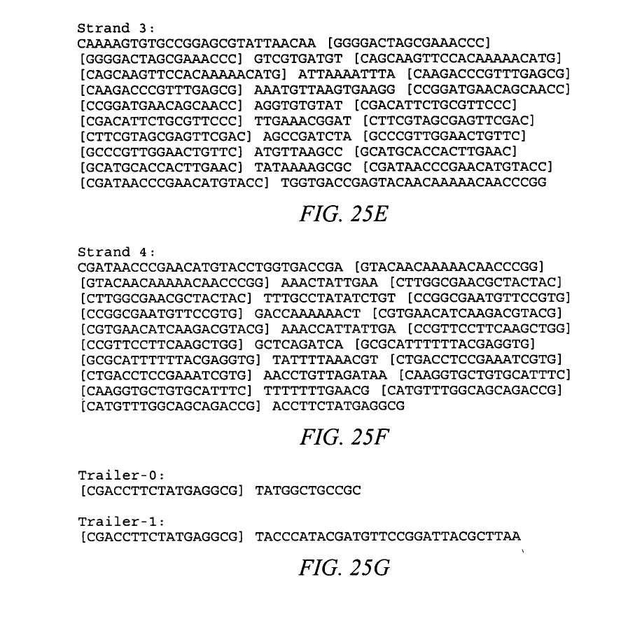

Overlap maps for the overlapping short segments used to build each of the five Intermediate Fragments 0-4, leaders, and trailers that make up Part II of the variola DNA polymerase gene are provided in FIG. 25A-FIG. 25G. SEQ. ID. NO.: 183 corresponds to leader-0 (FIG. 25A), SEQ. ID. NO.: 184 corresponds to leader-1 (FIG. 25A), SEQ. ID. NO.: 185-SEQ. ID. NO.: 196 correspond to strand-0 (FIG. 25B), SEQ. ID. NO.: 197-SEQ. ID. NO.: 208 correspond to strand-1 (FIG. 25C), SEQ. ID. NO.: 209-SEQ. ID. NO.: 218 correspond to strand-2 (FIG. 25D), SEQ. ID. NO.: 219-SEQ. ID. NO.: 228 correspond to strand-3 (FIG. 25E), SEQ. ID. NO.: 229-SEQ. ID. NO.: 238 correspond to strand-4 (FIG. 25F), SEQ. ID. NO.: 239 corresponds to trailer-0 (FIG. 25G), SEQ. ID. NO.: 240 corresponds to trailer-1 (FIG. 24G). As is described in greater detail below, each short segment shown in FIG. 25A-FIG. 25G was synthesized directly and assembled into the five Intermediate Fragments in five parallel reactions, then in a second stage, the five Intermediate Fragments were assembled into Part II of the variola DNA polymerase gene.

Biochemistry

The variola DNA polymerase gene was assembled in the reverse order of the design process: first, the assembly of the ten Intermediate Fragments (five each for Parts I and II of the variola DNA polymerase gene); second, the assembly of the two 1500 bp large pieces, Parts I and II (which combined make up the full length gene); and finally, Apa I digestion of Part I (in the segment 1Seg-4-09) and Part II (in the segment 2Seg-2-00) to generate compatible flanking termini, allowing the two large pieces to be ligated together to generate the full-length variola DNA polymerase gene.

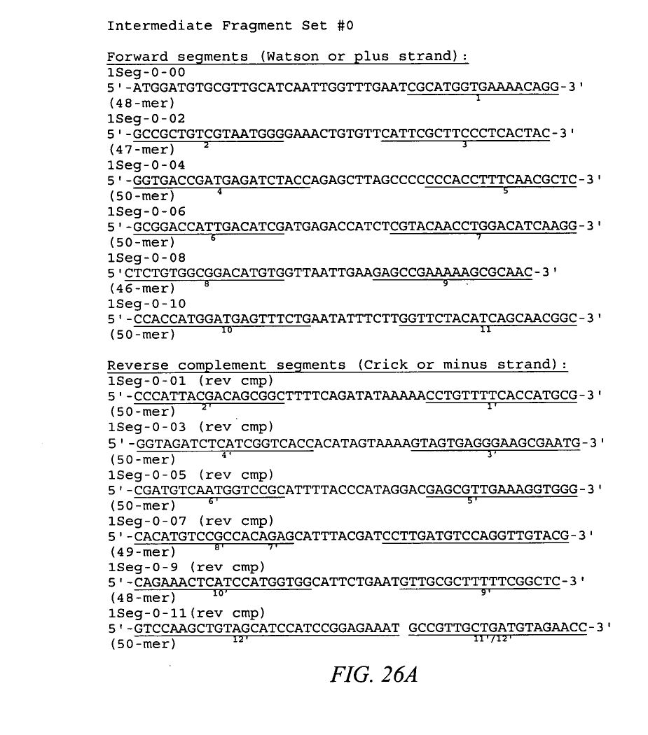

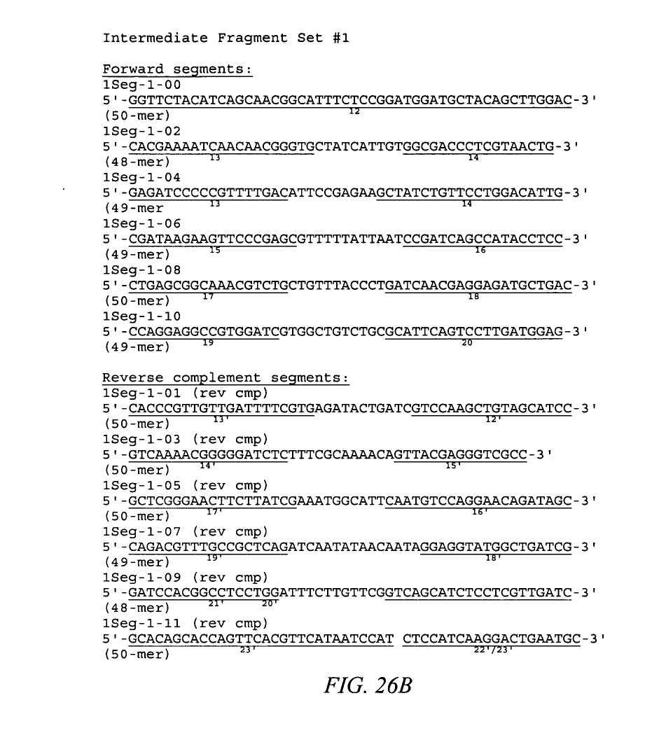

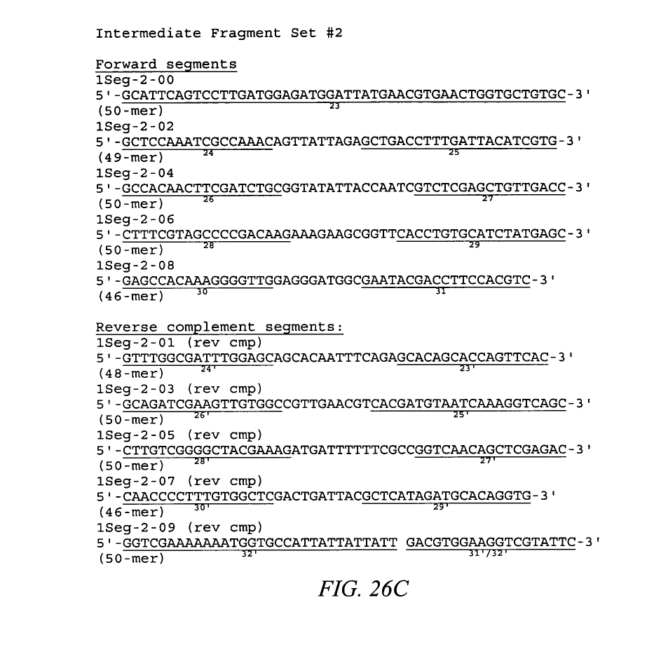

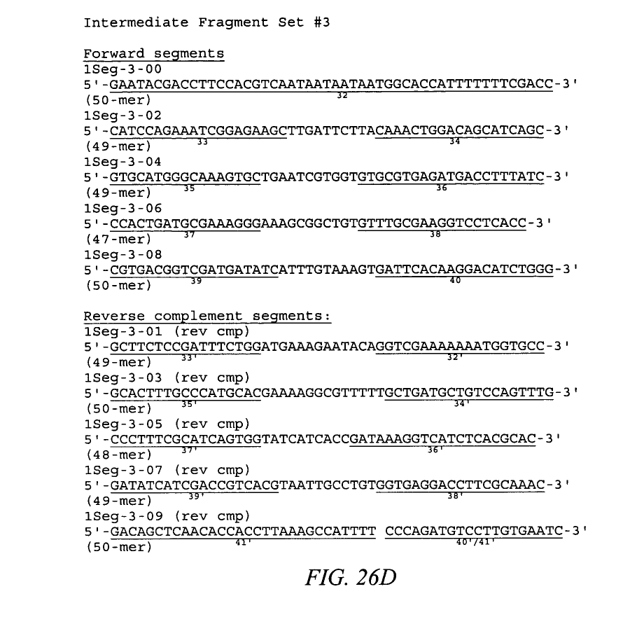

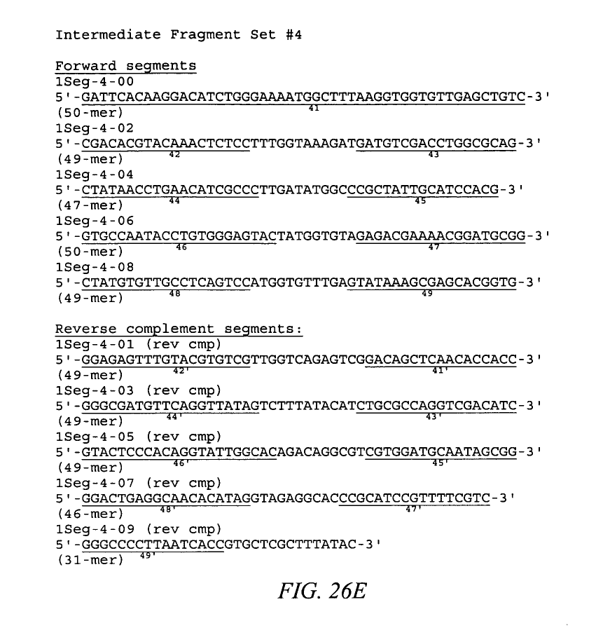

variola DNA polymerase Part I. Each of the five Intermediate Fragments 0-4 that make up Part I was assembled from synthetic oligonucleotide sets of alternating strand specificity that overlap one another. The sequences of the short ssDNA segments used to construct each of the Intermediate Fragments are provided in FIG. 26A-FIG. 26E. Overlaps between adjacent segments are indicated by underlining and identifying numbers beneath the underlined regions. Overlaps identified with primed numbers are complementary to the corresponding unprimed overlaps. SEQ. ID. NO.: 241-SEQ. ID. NO.: 252 correspond to 1Seg-0-00 through 1Seg-0-11 (FIG. 26A), SEQ. ID. NO.: 253-SEQ. ID. NO.: 264 correspond to 1Seg-1-00 through 1Seg-1-11 (FIG. 26B), SEQ. ID. NO.: 265-SEQ. ID. NO.: 274 correspond to 1Seg-2-00 through 1Seg-2-09 (FIG. 26C), SEQ. ID. NO.: 275-SEQ. ID. NO.: 284 correspond to 1Seg-3-00 through 1Seg-3-09 (FIG. 26D), and SEQ. ID. NO.: 285-SEQ. ID. NO.: 294 correspond to 1Seg-4-00 through 1Seg-4-09 (FIG. 26E).

For each of the Intermediate Fragments, the first segment (1Seg-0-00, 1Seg-1-00, 1Seg-2-00, 1Seg-3-00, and 1Seg-4-00)-serves as the leader primer for overlap extension. The last segment (1Seg-0-11, 1Seg-1-11, 1Seg-2-09, 1Seg-3-09, and 1Seg-4-09) serves as the trailer primer.

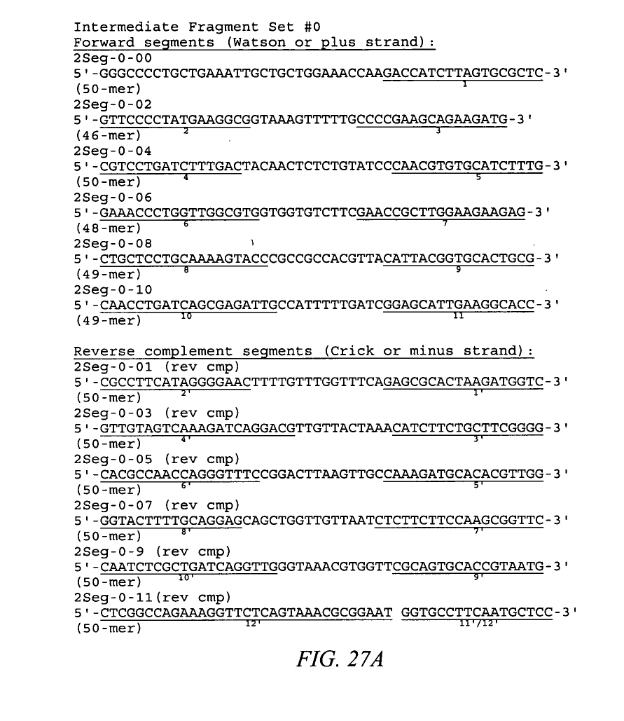

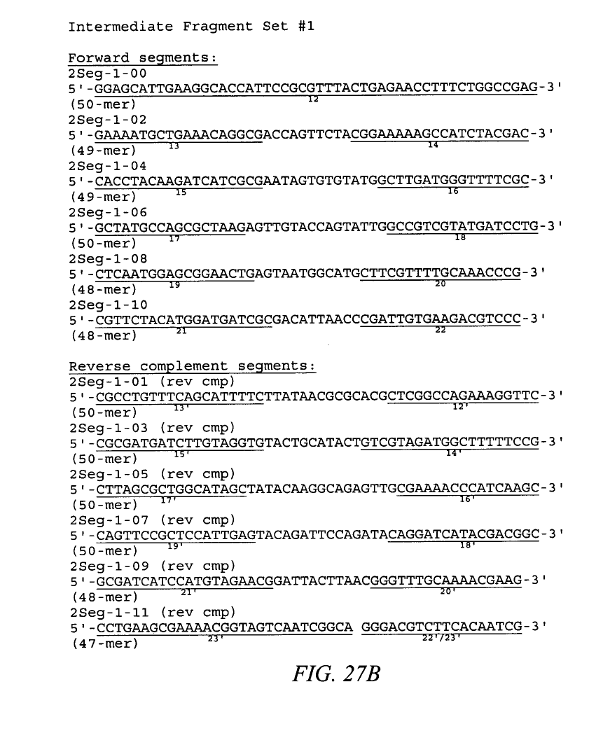

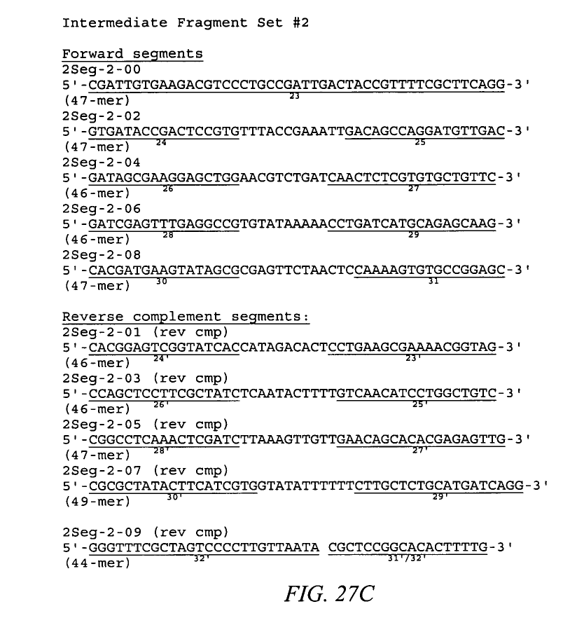

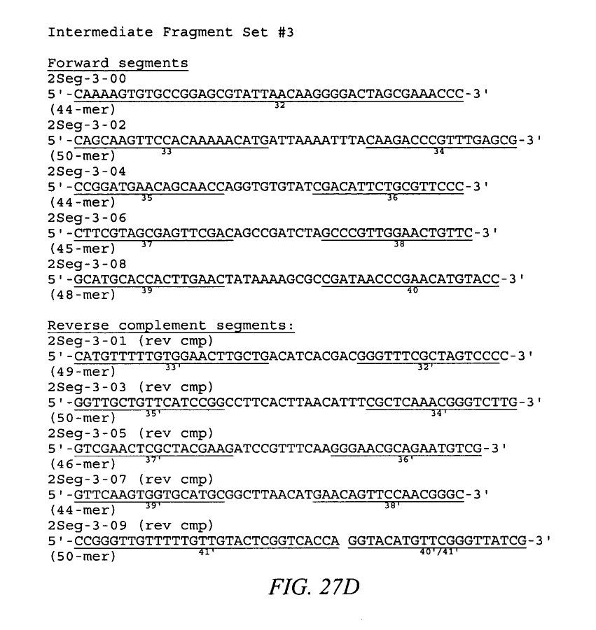

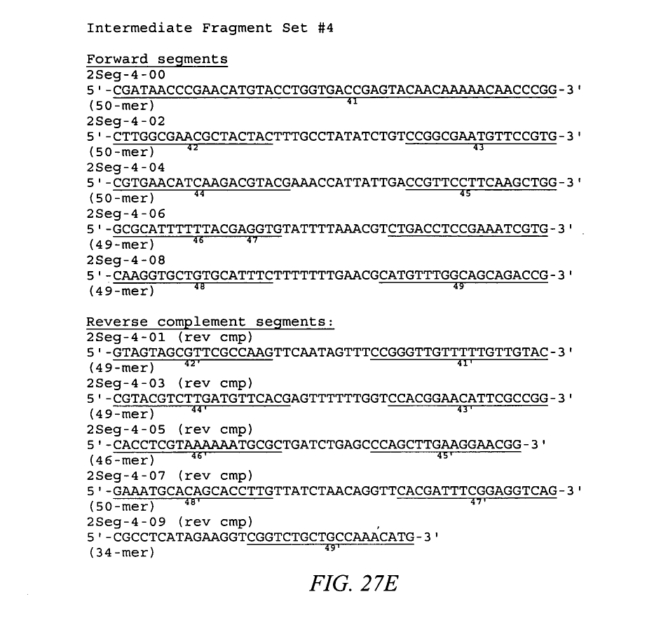

variola DNA polymerase Part II. Each of the five Intermediate Fragments 0-4 that make up Part II was assembled from synthetic oligonucleotide sets of alternating strand specificity that overlap one another. The sequences of the short ssDNA segments used to construct each of the Intermediate Fragments are provided in FIG. 27A-FIG. 27E. Overlaps between adjacent segments are indicated by underlining and identifying numbers beneath the underlined regions. Overlaps identified with primed numbers are complementary to the corresponding unprimed overlaps. SEQ. ID. NO.: 295-SEQ. ID. NO.: 306 correspond to 2Seg-0-00 through 2Seg-0-11 (FIG. 27A), SEQ. ID. NO.: 307-SEQ. ID. NO.: 318 correspond to 2Seg-1-00 through 2Seg-1-11 (FIG. 27B), SEQ. ID. NO.: 319-SEQ. ID. NO.: 328 correspond to 2Seg-2-00 through 2Seg-2-09 (FIG. 27C), SEQ. ID. NO.: 329-SEQ. ID. NO.: 338 correspond to 2Seg-3-00 through 2Seg-3-09 (FIG. 27D), and SEQ. ID. NO.: 339-SEQ. ID. NO.: 348 correspond to 2Seg-4-00 through 2Seg-4-09 (FIG. 27E).

For each of the Intermediate Fragments, the first segment (2Seg-0-00, 2Seg-1-00, 2Seg-2-00, 2Seg-3-00, and 2Seg-4-00) serves as the leader primer for overlap extension. The last segment (2Seg-0-11, 2Seg-1-11, 2Seg-2-09, 2Seg-3-09, and 2Seg-4-09) serves as the trailer primer.

Assembly of the Five Intermediate Fragments into Large Pieces Part I and Part II

Each Intermediate Fragment was separately constructed in a first overlap extension reaction from the appropriate set of ssDNA sequences provided in FIG. 26A-FIG. 26E and FIG. 27A-FIG. 27E. The reaction mixture for each reaction is provided in TABLE IX and the thermocycler program in TABLE X. The “synthetic oligonucleotide mix” entry in TABLE IX is a 27.5 ng/μL mixture of equal amounts of each of the forward and reverse complement synthetic oligonucleotides for a particular Intermediate Fragment.

TABLE IX

| Reagent | Quantity |
|---|---|
| Roche High Fidelity PCR Master, Vial #1 | 25.0 μL |
| (Product # 2140314) | |
| Synthetic oligonucleotide mix* | 1.0 μL |
| Leader primer, 50 μM | 0.5 μL |
| Trailer primer, 50 μM | 0.5 μL |
| Sterile water, Roche (molecular biology grade) | 23.0 μL |

*For each of the synthetic oligonucleotide sets (0-4), the forward and reverse complement synthetic oligonucleotides were mixed in equal amounts to a final concentration of 27.5 ng/μL.

TABLE X

| Step | Conditions |
|---|---|
| 1. | 94° C. for 5 minutes for initial denaturation, |
| 2. 20 cycles of: | 94° C. for 1 minute |
| | 50° C. for 30 seconds |
| | 65° C. for 5 minutes |
| 3. | 65° C. for 10 minutes for final extension |

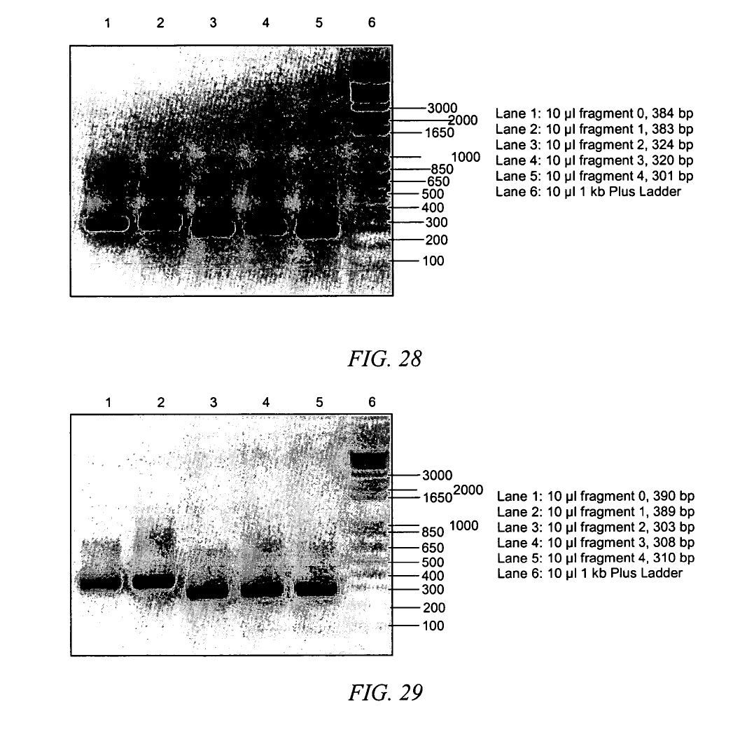

The products of these reactions were separated on 1% agarose gels as shown in FIG. 28 for Part I of the variola DNA polymerase gene and FIG. 29 for Part II.

Assembly of the Intermediate Fragments into Full Length Part I and Part II of the Variola DNA Polymerase Gene

Parts I and II of the variola DNA polymerase gene were assembled in a second overlap extension reaction of their constituent Intermediate Fragment sets using the reaction mixture provided in TABLE XI and the thermocycler program provided in TABLE XII.

TABLE XI

| Reagent | Quantity |
|---|---|
| Intermediate Fragment 0* | 0.5 μL |
| Intermediate Fragment 1* | 0.5 μL |
| Intermediate Fragment 2* | 0.5 μL |
| Intermediate Fragment 3* | 0.5 μL |
| Intermediate Fragment 4* | 0.5 μL |
| Leader primer, 50 μM | 1.0 μL |
| Trailer primer, 50 μM | 1.0 μL |
| Sterile water, Roche (molecular biology grade) | 20.5 μL |
| Roche High Fidelity PCR Master, Vial #1 | 25.0 μL |
| (Product # 2140314) | |

*Taken directly from overlap extension reactions (FIG. 28 and FIG. 29)

For the construction of Part I of the variola DNA polymerase gene, two overlap extension reactions were performed using the two different sets of primers illustrated in FIG. 24A and FIG. 24G. The first reaction used the thirty base 1lead-01 leader (5′-TCCTCGAGCATAAT GGATGTGCGTTGCATC-3′, SEQ. ID. NO.: 125) and the twenty-nine base 1trail-57 trailer (5′-GCGGCAGCCA TAGGGCCCCTTAATCACCG-3′, SEQ. ID. NO.: 349). The second reaction used the forty-eight base 1lead-02 leader 5′-GACGACGACGACAAGCATATGCTCGAGGATA TGGATGTGCGTTGCATC-3′, SEQ. ID. NO.: 126) and the forty-seven base 1trail-58 trailer (5′-TTAAGCGTAATCCGGAACATCGTATGGGTAGGGCCCCTTAATCACCG-3′, SEQ. ID. NO.: 350).

For the construction of Part II of the variola DNA polymerase gene, two overlap extension reactions were performed using two different sets of primers illustrated in FIG. 25A and FIG. 25G. The first reaction used the thirty base 2lead-01 leader (5′-TCCTCGAGCATAGGGCCCCTGCTGAAATTG-3′, SEQ. ID. NO.: 183) and the twenty-nine base 2trail-57 trailer (5′-GCGGCAGCCATACGCCTCATAGAAGGTCG-3′, SEQ. ID. NO.: 351). The second reaction used the forty-eight base 2lead-02 leader (5′-GACGACGACGACAAGCATATGCTCGAGGATG GGCCCCTGCTGAAATTG-3′, SEQ. ID. NO.: 184) and the forty-seven base 2trail-58 trailer (5′-TTAAGCGTA ATCCGGAACATCGTATGGGTACGCCTCATAGAAGGTCG-3′, SEQ. ID. NO.: 352).

TABLE XII

| Step | Conditions |
|---|---|
| 1. | 94° C. for 5 minutes for initial denaturation, |
| 2. 20 cycles of: | 94° C. for 1 minute |
| | 60° C. for 30 seconds |
| | 65° C. for 10 minutes |
| 3. | 65° C. for 10 minutes for final extension |

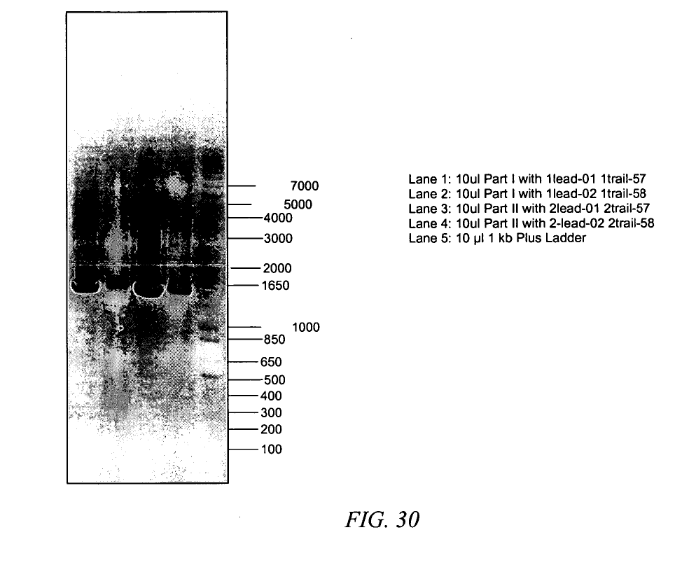

The products of these reactions were separated on a 1% agarose gel, which is illustrated in FIG. 30.

Ligation of Part I and Part II to Generate the Full Length Variola DNA Polymerase Gene (3000 bp)

The DNA from the lanes corresponding to Parts I and II of the variola DNA polymerase gene was purified using GENECLEAN® (BIO101 Systems®, Qbiogene). The two fragments were separately digested overnight with Apa I (Boehringer Mannheim), purified on a 1% agarose gel, and purified using GENECLEAN®.

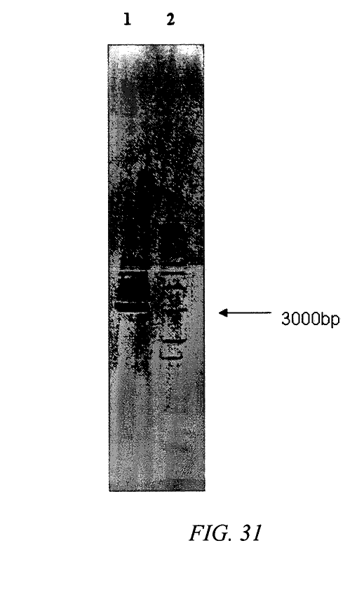

Parts I and II were ligated for 1.25 hr at ambient temperature under the conditions provided in TABLE XIII. The products were quantified by fluorometry. Ligation was confirmed by PCR using the 1lead-01 and 2trail-57 primers, the product of which was isolated on a 1% agarose gel, shown in FIG. 31. The PCR product was run in lane 1 and 1 kB Plus ladder in lane 2.

TABLE XIII

| Reagent | Quantity |
|---|---|
| Water | 15 μL |
| Part I (22 ng/μL) | 1 μL |
| Part II (22 ng/μL) | 1 μL |
| T4 10 × ligation buffer | 2 μL |
| T4 ligase (400 U/μL) | 1 μL |

### EXAMPLE 3

### E. coli Threonine Deaminase by One-Step Hierarchical Decomposition and Ligation

EXAMPLE 3 illustrates the synthesis of an E. coli threonine deaminase gene by dividing the gene in a one-step hierarchical decomposition and synthesizing the gene by the direct self-assembly method.

Design

In the one-step hierarchical decomposition, the gene was divided directly into 54-overlapping short segments, in the present example not longer than about 60 bases, overlap not shorter than about 27 bases. Because the gene was designed for reassembly by ligation, the adjacent short segments on the same strand abut, i.e., with no single-stranded gaps between the double-stranded overlaps. The overlaps were not designed to terminate in a G or C.

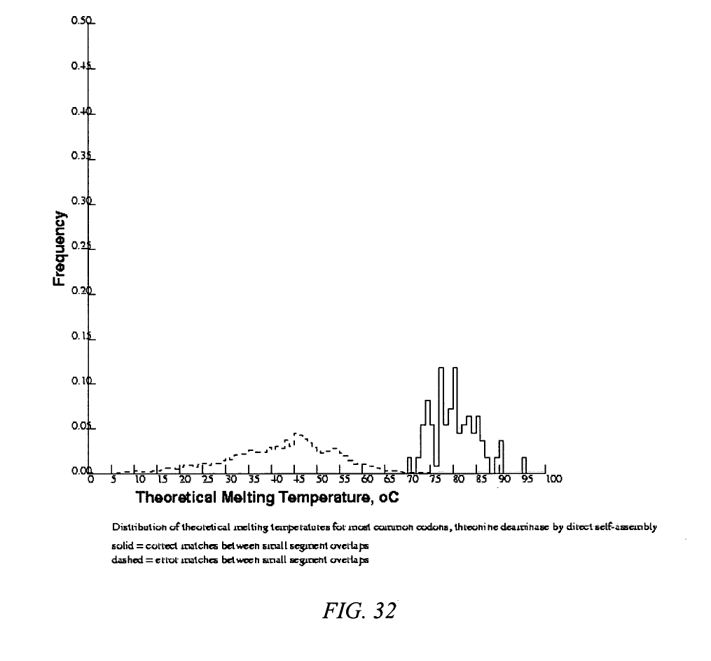

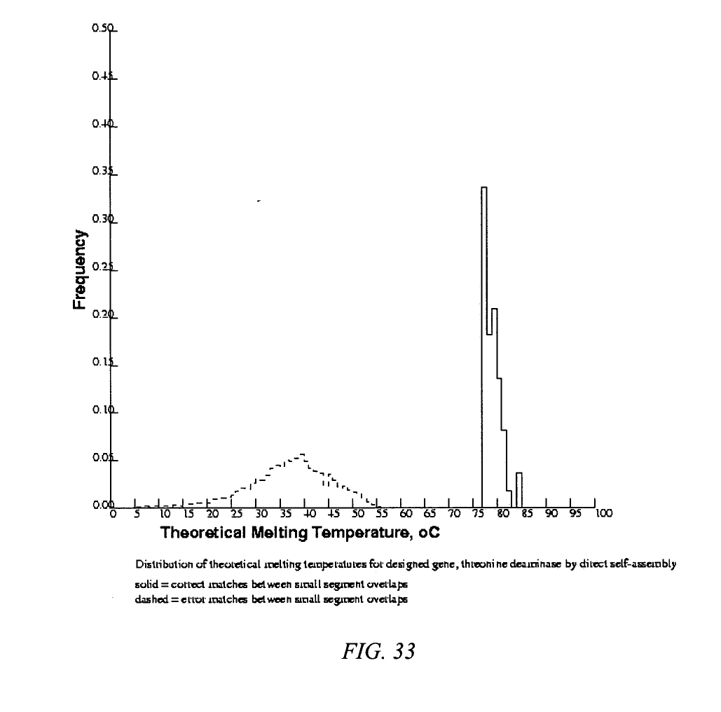

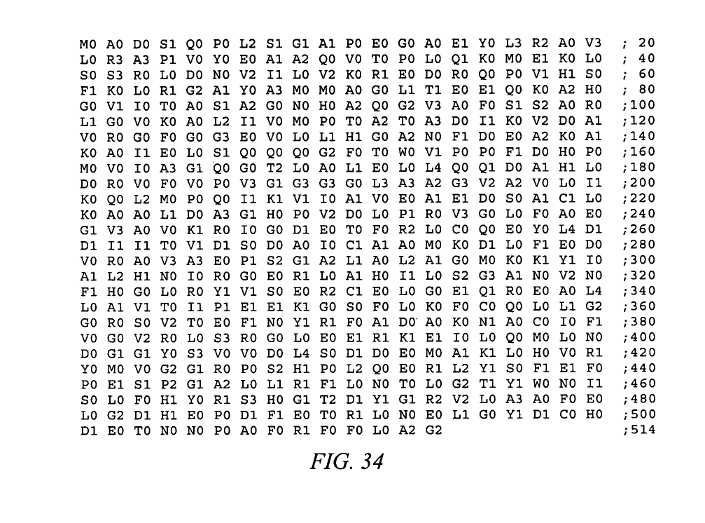

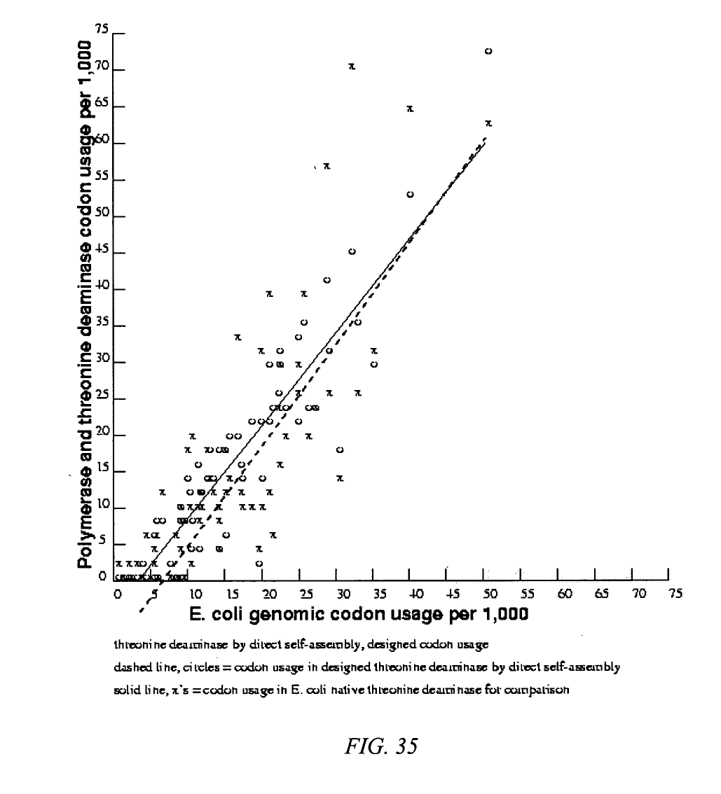

Theoretical melting temperatures were calculated as described in EXAMPLE 1. The distribution of calculated melting temperatures of the short segments using the most common E. coli codons is provided in FIG. 32. The codons were permuted to increase the gap in the melting temperatures as described above, resulting in the calculated melting temperatures for the final short segments provided in FIG. 33. The calculated temperature gap in this example was 21.6° C. (77.1° C.-55.5° C.). The final sequence for the synthetic E. coli threonine deaminase gene is provided in FIG. 34 (SEQ. ID. NO.: 353) using the codon key from FIG. 9 of EXAMPLE 1. A comparison of the codon usage of the designed threonine deaminase gene compared with that of the native gene is illustrated in FIG. 35, indicating the similarity in codon frequency.

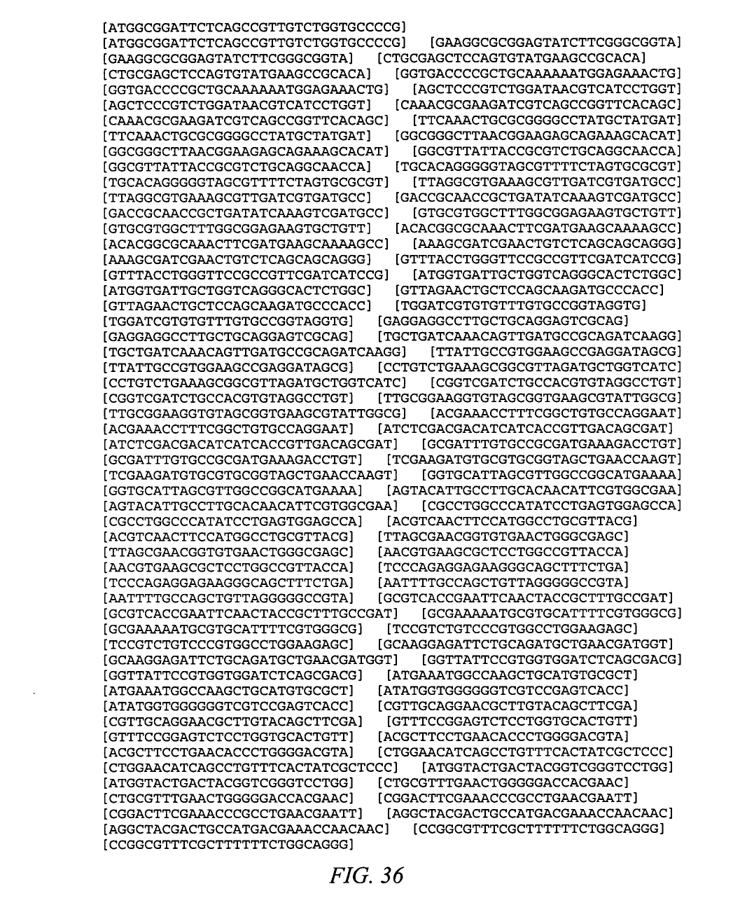

The resulting DNA sequence was decomposed into the 54 short overlapping segments shown in an overlap map in FIG. 36 (SEQ. ID. NO.: 354-SEQ. ID. NO.: 407). Each short segment in FIG. 36 was synthesized directly and assembled into the synthetic gene in one reaction step.

Biochemistry

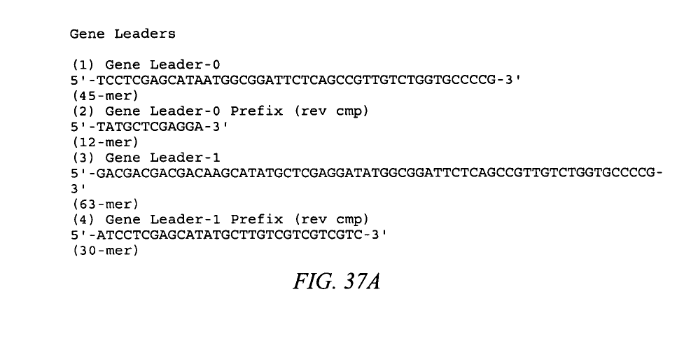

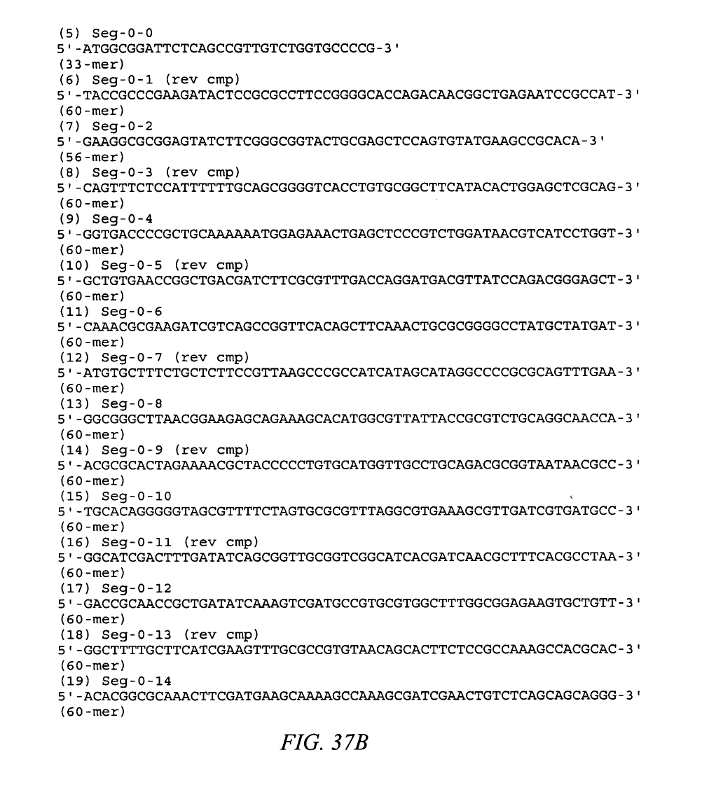

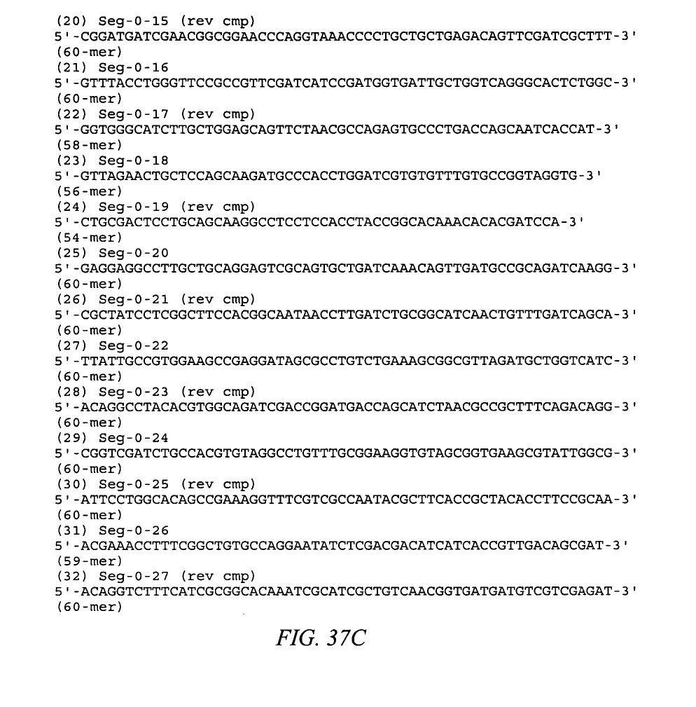

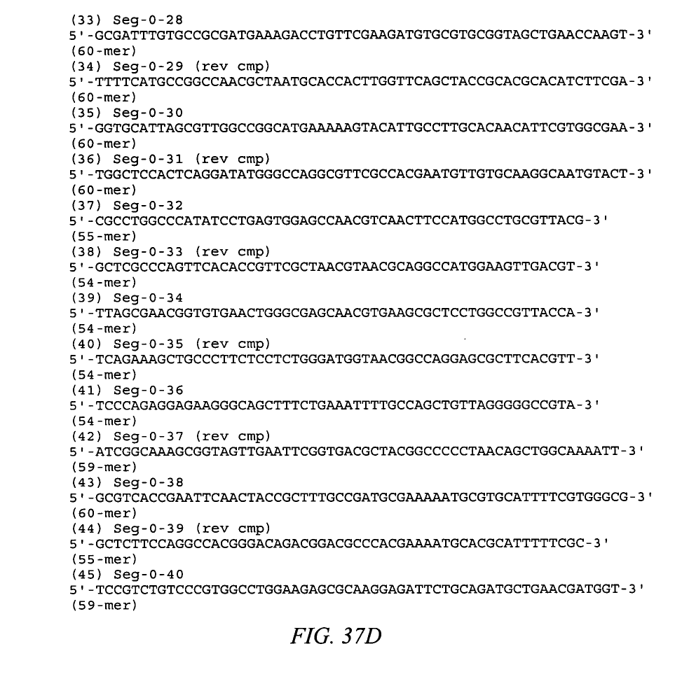

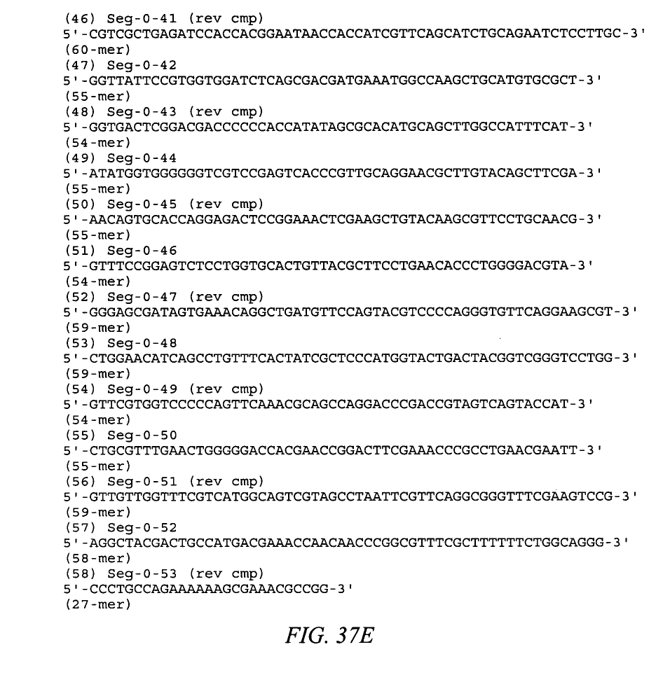

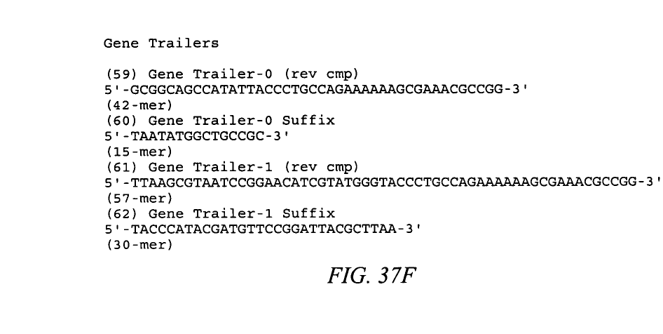

The threonine deaminase gene was assembled in two steps by direct self-assembly. The sequences of the leaders, short ssDNA segments, and trailers are provided in FIG. 37A-FIG. 37F. SEQ. ID. NO.: 408-SEQ. ID. NO.: 411 correspond to the gene leaders illustrated in FIG. 37A, SEQ. ID. NO.: 412-SEQ. ID. NO.: 465 correspond to Seg-0-0 through Seg-0-53 (FIG. 37B-FIG. 37E), and SEQ. ID. NO.: 466-SEQ. ID. NO.: 469 correspond to the gene trailers illustrated in FIG. 37F. Overlaps between adjacent segments are not indicated. The DNA segments were purchased from a commercial source.

The threonine deaminase gene was first divided into four Medium-sized pieces of equal size, which were each reassembled by direct self-assembly and ligation, then the four Medium-sized pieces were combined and ligated into the full-length gene.

Direct Self-Assembly of the Four Medium-Sized Pieces

Each of the four Medium-sized pieces was constructed in parallel as follows.

Annealing Reaction. The short segments were first annealed in a thermocycler to form DNA constructs corresponding to the Medium-sized pieces. Each medium-sized piece was divided into two parts, a forward strand and a reverse strand. For each strand, a 20 μL solution of the short segments corresponding to each strand at a concentration of 0.825 μM was treated with 800 U of T4 polynucleotide kinase. The kinased, short segments corresponding to the forward and reverse strands of each Medium-sized piece were mixed together. A phenol extraction was performed on the mixture, followed by an ethanol precipitation and a 70% ethanol wash. The pellet was resuspended in 7.8 μL of 1×TE, from which a solution detailed in TABLE XIV was prepared. The concentration of each short segment was 1.65 μM in this solution. This solution was placed in a thermocycler, which was programmed as described in TABLE XV.

TABLE XIV

| Reagent | Quantity |
|---|---|
| Synthetic oligonucleotide mix | 7.8 μL |
| NaCl, 5 M | 0.2 μL |
| MgCl2, 1 M | 1.0 μL |
| Nuclease free water | 1.0 μL |

TABLE XV

| Step | Conditions |
|---|---|
| 1. | 94° C. for 5 minutes for initial denaturation, |
| 2. | 80° C. for 1 minute |
| 3. | Cool to 55° C. at 0.5° C./min |
| 4. | 4° C. |

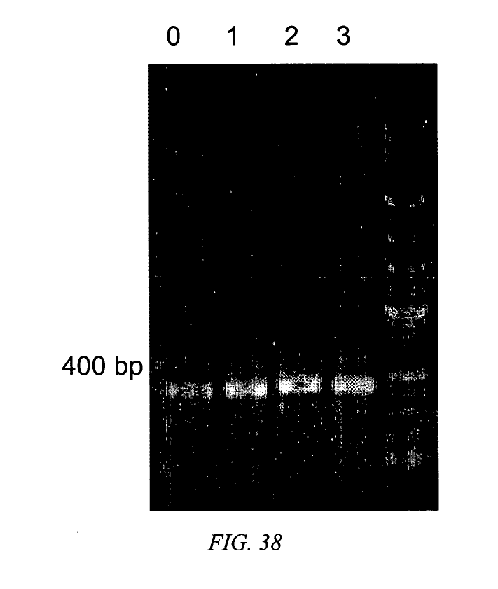

Ligation Reaction. Each Medium-sized piece was produced by ligation of the corresponding DNA construct synthesized in the annealing reaction, described above. The reaction mixture provided in TABLE XVI was maintained at 16° C. overnight. An agarose gel of the resulting four Medium-sized pieces is provided in FIG. 38.

TABLE XVI

| Reagent | Quantity |
|---|---|
| DNA construct solution from annealing | 10.0 μL |
| reaction | |
| T4 DNA Ligase, 400 U/mL | 2.0 μL |
| 10 × Ligase Buffer | 2.0 μL |
| Nuclease free water | 6.0 μL |

Assembly of the Four Medium-Sized Pieces into the Threonine Deaminase Gene

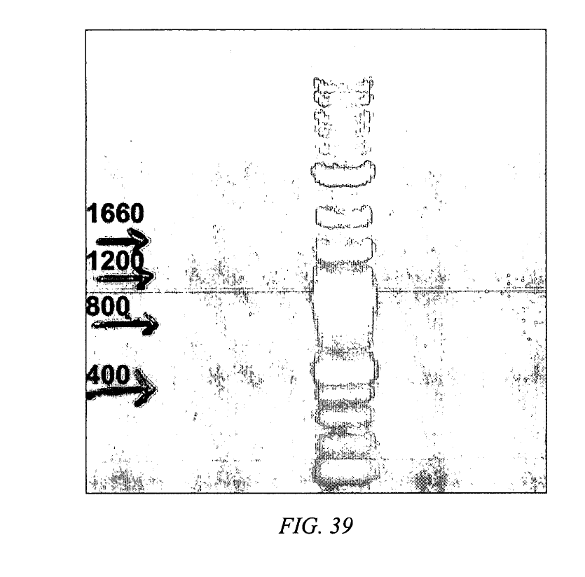

Each Medium-sized piece was isolated from the gel using GENECLEAN® (BIO101 Systems®, Qbiogene). A ligation reaction mixture containing the four Medium-sized pieces is provided in TABLE XVII. The ligation reaction was performed at 16° C. overnight. An agarose gel of the products, including the full-length threonine deaminase gene, is provided in FIG. 39.

TABLE XVII

| Reagent | Quantity |
|---|---|
| DNA construct solution from annealing | 10.0 μL |
| reaction | |
| T4 DNA Ligase, 400 U/mL | 2.0 μL |
| 10 × Ligase Buffer | 2.0 μL |
| Nuclease free water | 6.0 μL |

At this point the assembled gene may be stored at 4° C. or cloned into expression vector pET14b (Novagen) by ExoIII digestion. The threonine deaminase gene is designed to have 12 bp 3′-end overhangs which are compatible to 5′-overhang regions of the pET14b vector after it has been treated with ExoIII for 1 minute at 14° C. The insert and vector are ligated by mixing and heating, followed by cooling to a temperature below Tm for the overlapping regions of the insert and vector. The annealed fragments are transformed into an E. coli host at 37° C.

### EXAMPLE 4

### Ty3 GAG3 by Two-Step Recursive Decomposition with Sampling and Sequencing

EXAMPLE 4 illustrates the synthesis of GAG3 by two-step recursive decomposition with sampling and sequencing. The GAG3 open reading frame (ORF) is 876 bp long.

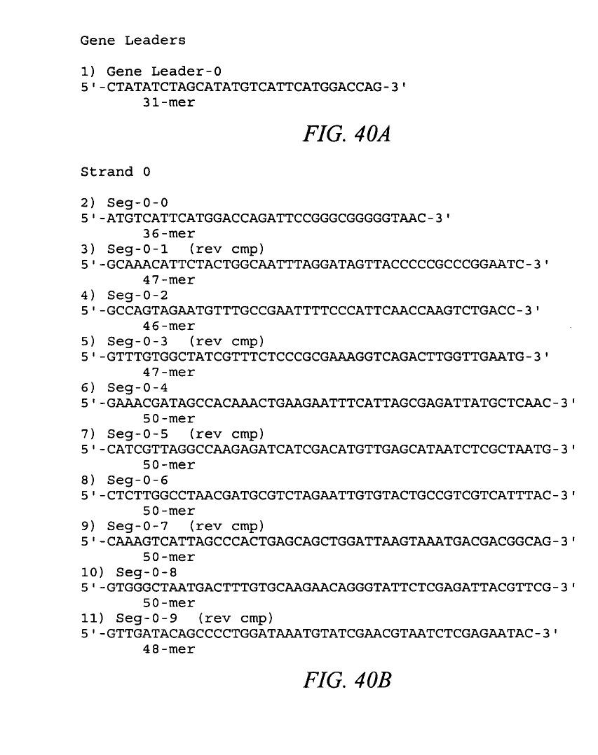

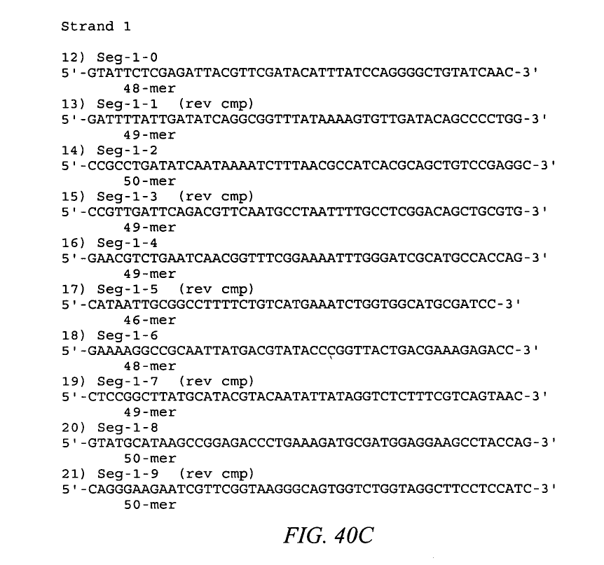

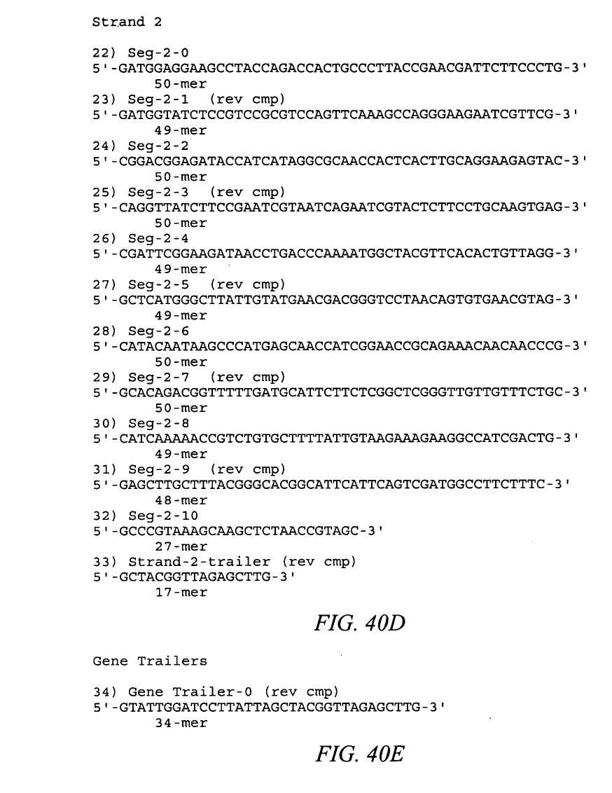

GAG3 ORF was divided into three overlapping intermediate fragments for reassembly by overlap extension. FIG. 40A-FIG. 40E illustrate the sequences of the gene leader, Fragments 0-2, and gene trailer. Fragment 0 was 307 bp, Fragment 1 was 324 bp, and Fragment 2 was 343 bp. Each intermediate fragment overlapped the adjacent one by 38 nt. Each intermediate fragment was divided into 10 oligonucleotides (50 nt) for reassembly by overlap extension. Adjacent oligonucleotides overlapped by about 19 bps. Collectively, the assembled sequence encoded both strands of each of the intermediate fragments. SEQ. ID. NO.: 470 corresponds to gene leader-0 (FIG. 40A), SEQ. ID. NO.: 471-SEQ. ID. NO.: 480 correspond to Seg-0-0 through Seg-0-9 (FIG. 40B), SEQ. ID. NO.: 481-SEQ. ID. NO.: 490 correspond to Seg-1-0 through Seg-1-9 (FIG. 40C), SEQ. ID. NO.: 491-SEQ. ID. NO.: 501 correspond to Seg-2-0 through Seg-2-10 (FIG. 40D), SEQ. ID. NO.: 502 corresponds to strand 2 trailer (FIG. 40D), and SEQ. ID. NO.: 503 corresponds to gene trailer-0 (FIG. 40E).

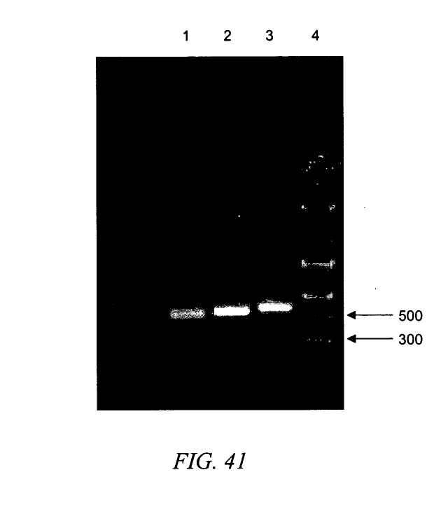

In the assembly of the intermediate fragments, the oligonucleotides were mixed to a final concentration of 0.1 μM with DNA polymerase (Proofstart®, Qiagen) and appropriate leader and trailer sequences. FIG. 41 is an agarose gel showing the products of these reactions. Fragment 0 is in lane 1, Fragment 1 in lane 2, and Fragment 2 in lane 3. Lane 4 contains a 2-Log DNA molecular weight ladder (New England Biolabs).

The intermediate fragments were each cloned using a blunt-end ligation procedure (pCR-Blunt II-TOPO® vector, Invitrogen). Four clones of each intermediate fragment were sequenced and correct sequence was selected. The selected sequences were amplified out of the vector by PCR (Proofstart® DNA polymerase, Qiagen).

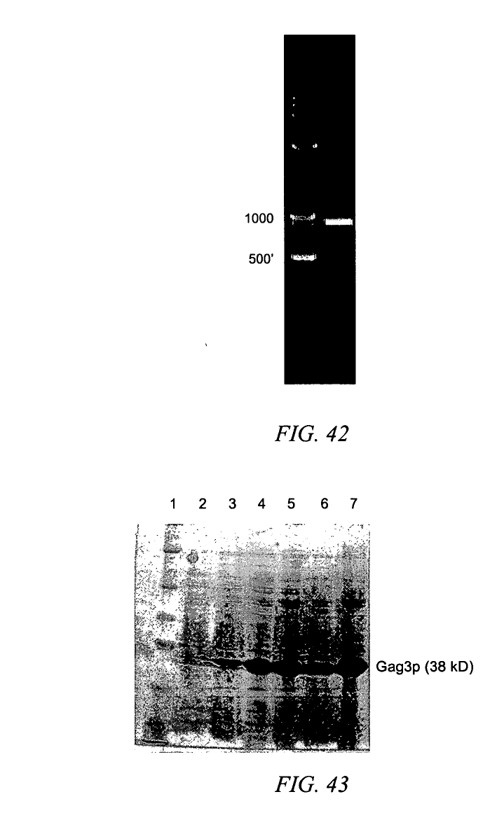

The intermediate fragments were mixed and extended to full-duplex DNA as described for the oligonucleotides. FIG. 42 is an agarose gel of the full-length GAG3 gene. The identity and accuracy of the gene was verified by sequencing both strands of the DNA product.

The synthetic GAG3 gene was cloned into the pET-3a plasmid (Novagen) using Nde I and BamH I endonuclease restriction sites designed in the 5′ and 3′ PCR gene primers. The resulting plasmid contained the entire GAG3 gene under the control of an inducible T7 promoter, and a bacterial ribosome-binding site (Shine-Dalgarno sequence). The BL21(DE3) pLysS strain of E. coli (Novagen) was transformed with the plasmid. T7 RNA polymerase expression was induced using host-encoded isopropyl-1-thio-β-D-galactopyranoside at a concentration of 0.4 mM. At 30 min intervals, cells were harvested by centrifugation and sonicated. FIG. 43 is an SDS-PAGE gel of the sonicate stained with Coomassie indicating the expression of Gag3p.

### EXAMPLE 5

### Ty3 IN Gene by Two-Step Recursive Decomposition with Sampling and Sequencing

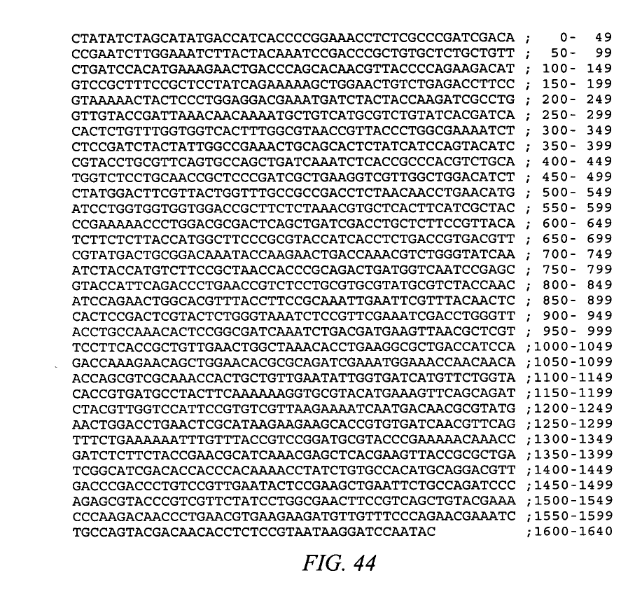

EXAMPLE 5 illustrates the synthesis of the Ty3 IN gene by a two-step recursive decomposition with sampling and sequencing. The Ty3 IN gene is 1640 bp long and is illustrated in FIG. 44 (SEQ. ID. NO.: 504).

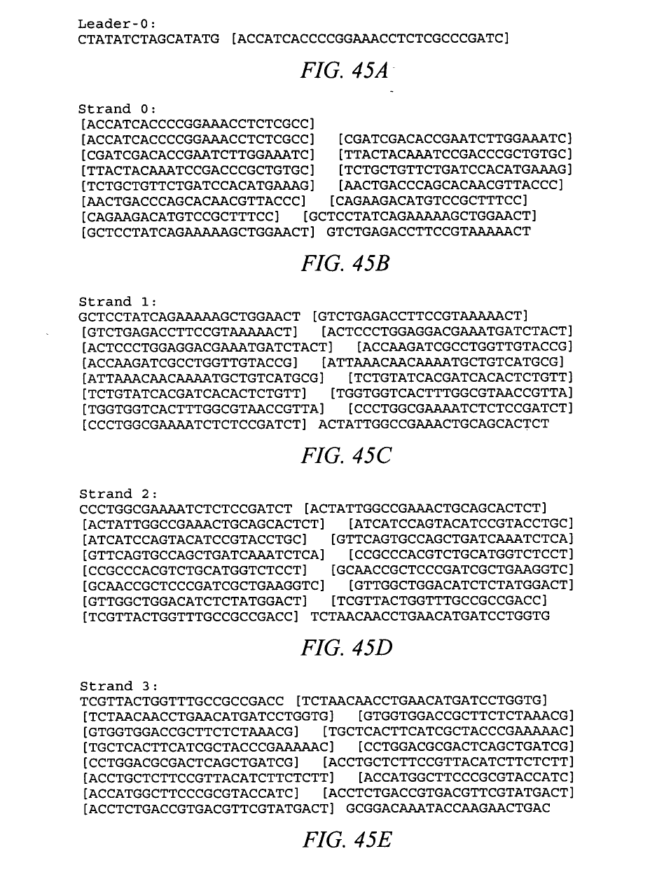

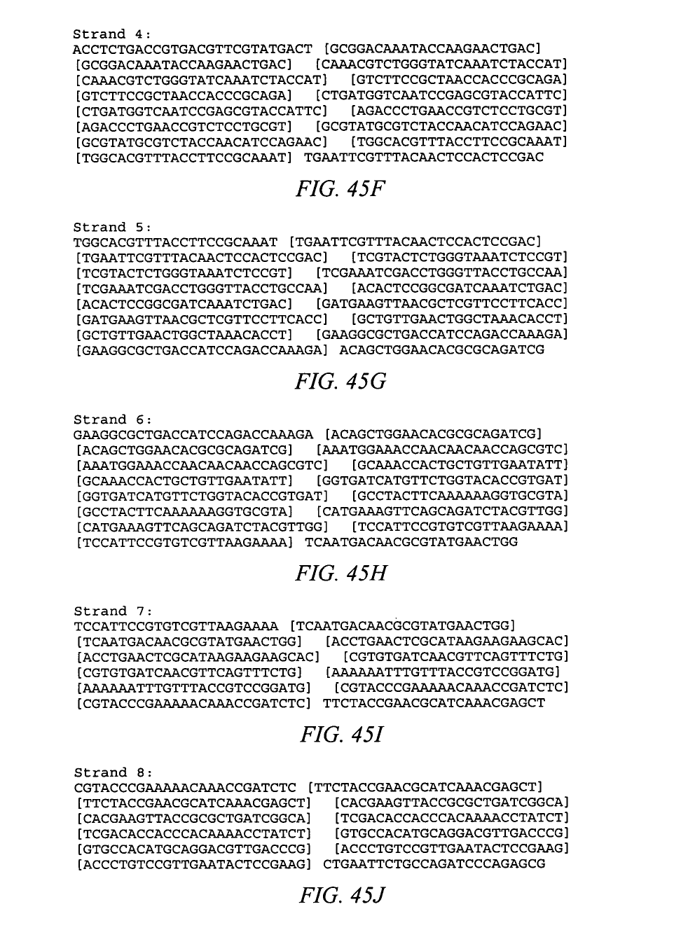

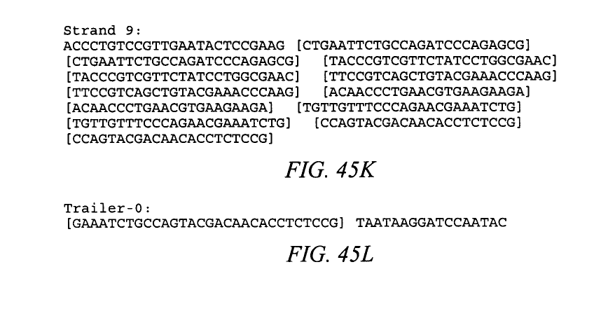

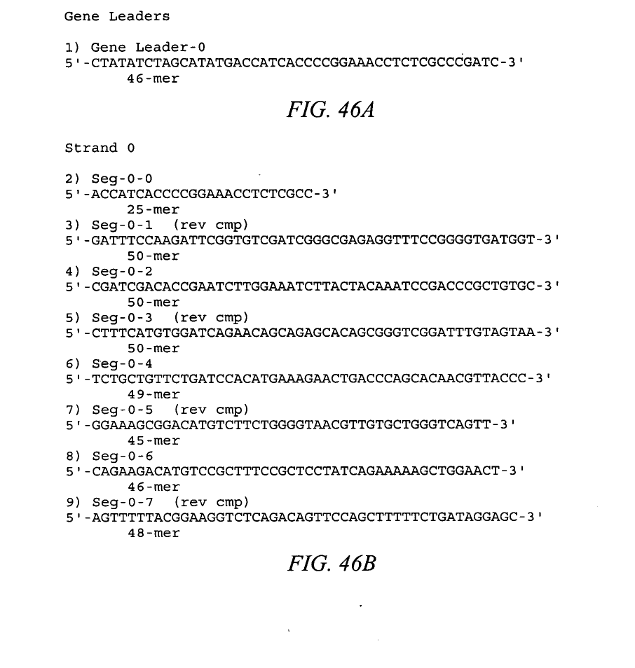

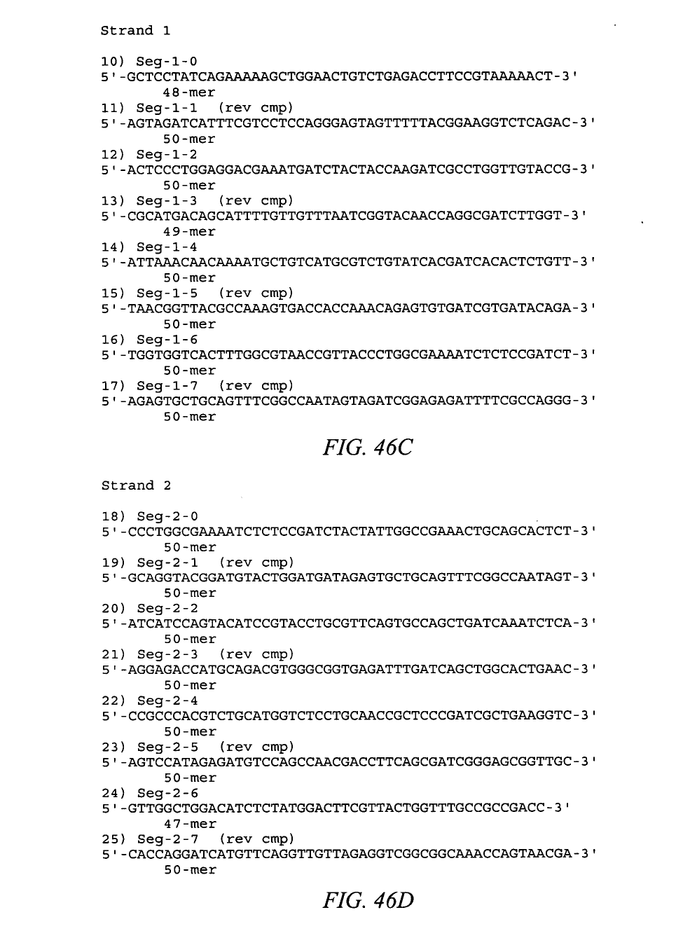

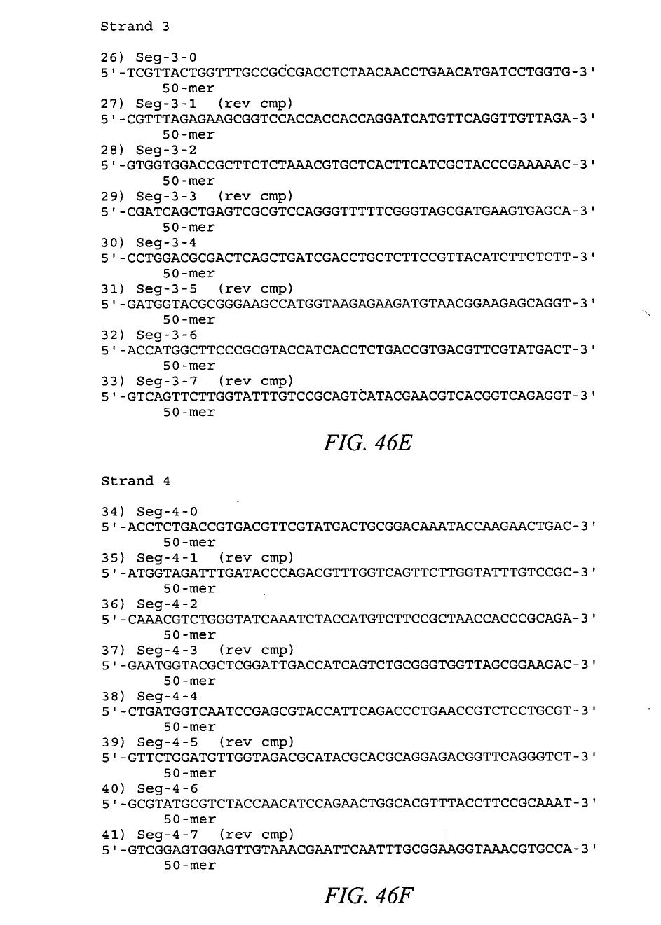

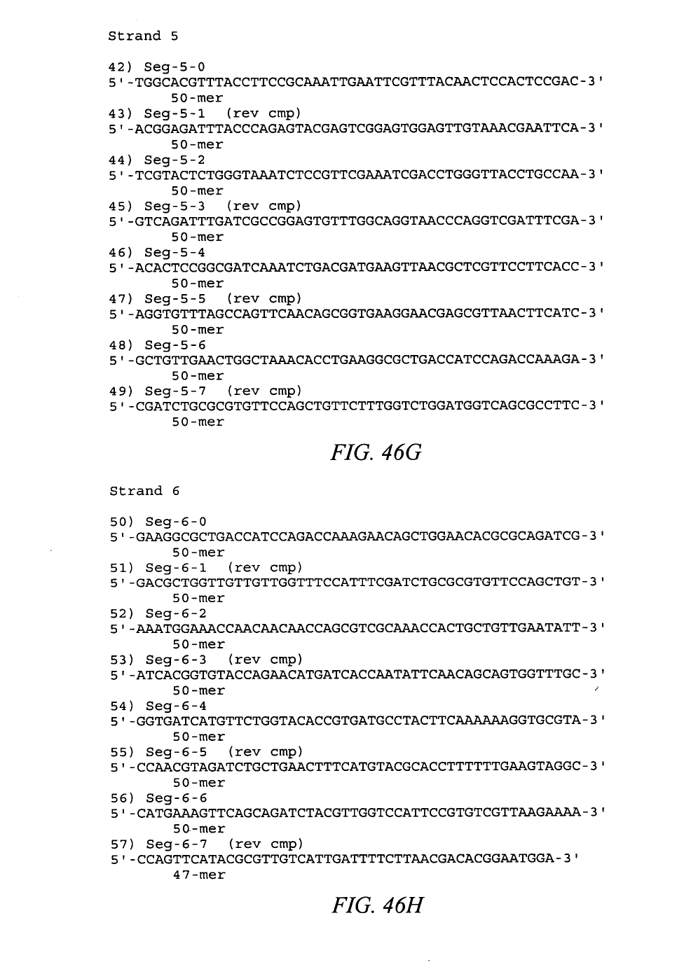

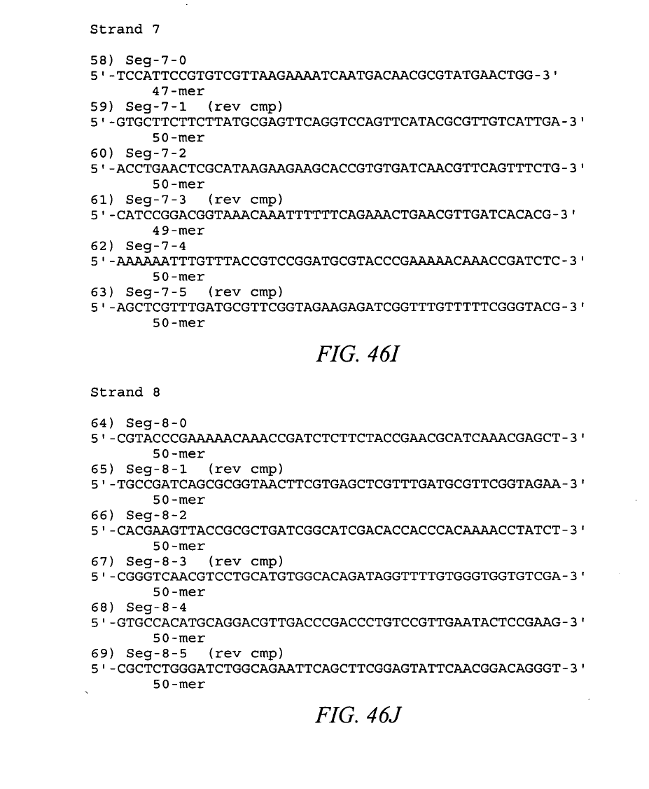

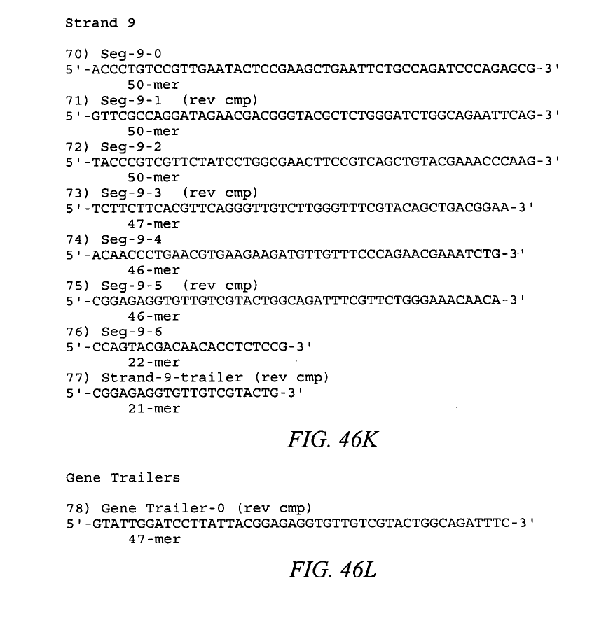

The Ty3 IN gene was divided into ten intermediate fragments for reassembly by overlap extension. Overlap maps for the leader, ten intermediate fragments, and trailer are provided in FIG. 45A-FIG. 45L: leader (FIG. 45A, SEQ. ID. NO.: 505), Fragment 0 (196 bp, FIG. 45B, SEQ. ID. NO.: 506-SEQ. ID. NO.: 513), Fragment 1 (224 bp, FIG. 45C, SEQ. ID. NO.: 514-SEQ. ID. NO.: 521), Fragment 2 (224 bp, FIG. 45D, SEQ. ID. NO.: 522-SEQ. ID. NO.: 529), Fragment 3 (223 bp, FIG. 45E, SEQ. ID. NO.: 530-SEQ. ID. NO.: 537), Fragment 4 (227 bp, FIG. 45F, SEQ. ID. NO.: 538-SEQ. ID. NO.: 545), Fragment 5 (223 bp, FIG. 45G, SEQ. ID. NO.: 546-SEQ. ID. NO.: 553), Fragment 6 (224 bp, FIG. 45H, SEQ. ID. NO.: 554-SEQ. ID. NO.: 561), Fragment 7 (172 bp, FIG. 451, SEQ. ID. NO.: 562-SEQ. ID. NO.: 567), Fragment 8 (175 bp, FIG. 45J, SEQ. ID. NO.: 568-SEQ. ID. NO.: 573), Fragment 9 (174 bp, FIG. 45K, SEQ. ID. NO.: 574-SEQ. ID. NO.: 580), and trailer (FIG. 45L, SEQ. ID. NO.: 581). Each of the intermediate fragments was divided into 50 nt oligonucleotides for reassembly by direct self-assembly illustrated in FIG. 46A-FIG. 46L. SEQ. ID. NO.: 582 corresponds to the gene leader (FIG. 46A), SEQ. ID. NO.: 583-SEQ. ID. NO.: 590 correspond to Seg-0-0 through Seg-0-7 (FIG. 46B), SEQ. ID. NO.: 591-SEQ. ID. NO.: 598 correspond to Seg-1-0 through Seg-1-7 (FIG. 46C), SEQ. ID. NO.: 599-SEQ. ID. NO.: 606 correspond to Seg-2-0 through Seg-2-7 (FIG. 46D), SEQ. ID. NO.: 607-SEQ. ID. NO.: 614 correspond to Seg-3-0 through Seg-3-7 (FIG. 46E), SEQ. ID. NO.: 615-SEQ. ID. NO.: 622 correspond to Seg-4-0 through Seg-4-7 (FIG. 46F), SEQ. ID. NO.: 623-SEQ. ID. NO.: 630 correspond to Seg-5-0 through Seg-5-7 (FIG. 46G), SEQ. ID. NO.: 631-SEQ. ID. NO.: 638 correspond to Seg-6-0 through Seg-6-7 (FIG. 46H), SEQ. ID. NO.: 639-SEQ. ID. NO.: 644 correspond to Seg-7-0 through Seg-7-5 (FIG. 461), SEQ. ID. NO.: 645-SEQ. ID. NO.: 650 correspond to Seg-8-0 through Seg-8-5 (FIG. 46J), SEQ. ID. NO.: 651-SEQ. ID. NO.: 657 correspond to Seg-9-0 through Seg-9-6 (FIG. 46K), SEQ. ID. NO.: 658 corresponds to the strand 9 trailer (FIG. 46K), and SEQ. ID. NO.: 659 corresponds to the gene trailer (FIG. 46L).

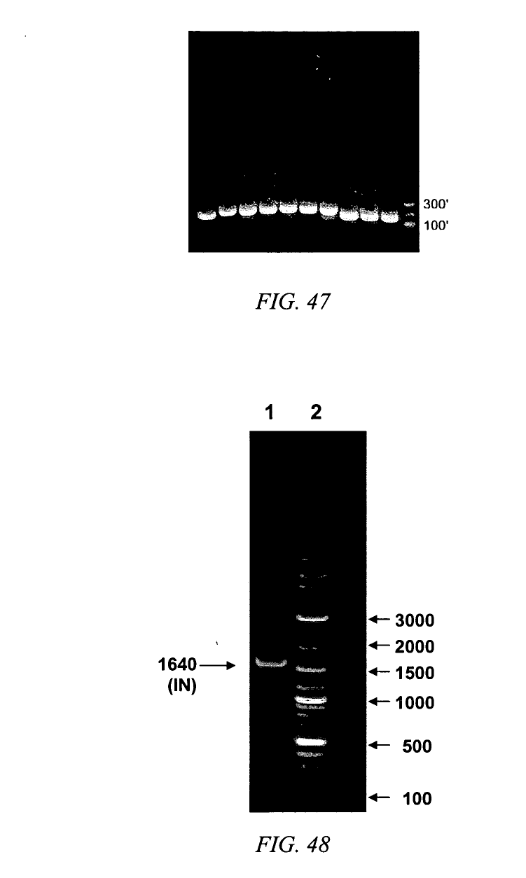

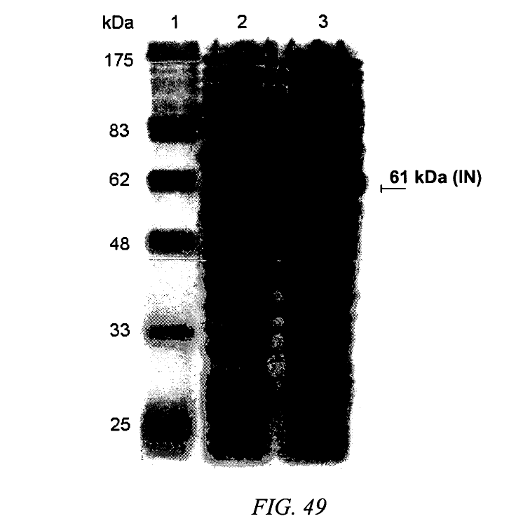

The ten intermediate fragments were separately assembled from the oligonucleotides, cloned, and sequenced as described in EXAMPLE 4. FIG. 47 is an agarose gel showing the products of the ten intermediate fragment reactions. The ten intermediate fragments were reassembled into the Ty3 IN gene using overlap extension as described in EXAMPLE 4. An agarose gel of the product is provided in FIG. 48. Lane 1 is the synthetic Ty3 IN gene, and lane 2, DNA size markers. The synthetic TY3 IN gene was cloned and expressed as described in EXAMPLE 4. An SDS-PAGE gel of the Coomassie stained TY3 IN protein is provided in FIG. 49. Lane 1 contains molecular weight markers; lane 2, uninduced cellular extract; and lane 3, induced cellular extract.

### EXAMPLE 6

### DNA Sequence Selection Using an α-Fragment Frameshifted Vector

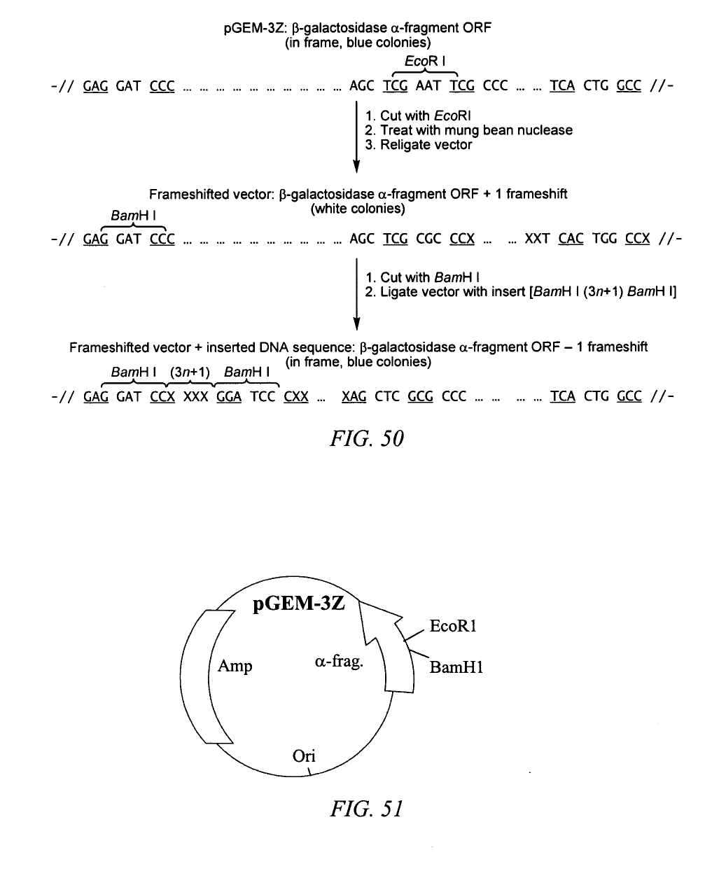

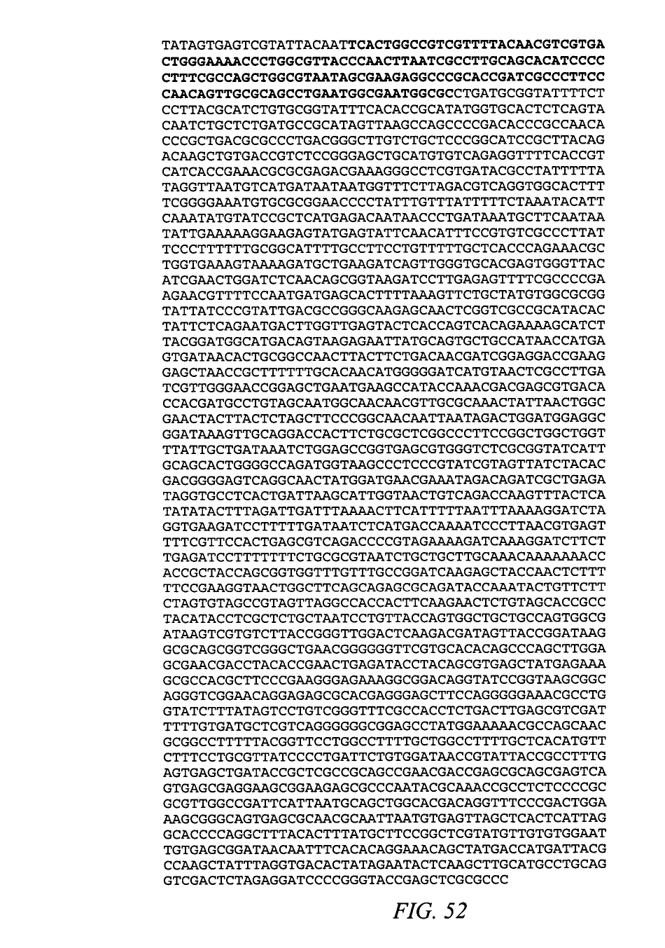

An α-fragment frameshifted vector was constructed as illustrated in FIG. 50 from a pGEM®-3Z vector (SEQ. ID. NO.: 660, Promega Corp. Madison, Wis.), which is schematically illustrated in FIG. 51. The gene for the α-complementing fragment of α-galactosidase is designated “α-frag.” EcoR I and BamH I restriction sites in the multiple cloning site (MCS) of the α-fragment fragment ORF are also indicated. An ampicillin resistance gene is designated “Amp.” Returning to FIG. 50, the pGEM®-3Z vector was cut at the EcoR I restriction endonuclease site, thereby linearizing the vector. The four 5′-overhang bases from each end of the linearized vector were removed using mung bean nuclease, and the vector religated to provide a α-fragment frameshifted vector (SEQ. ID. NO.: 661). The reverse complement of the α-fragment frameshifted vector is illustrated in FIG. 52 (SEQ. ID. NO.: 662) with the gene for the α-complementing fragment is in boldface.

The resulting frameshifted vector was transformed into E. coli JM109 (genotype: e14-(McrA-) recA1 endA1 gyrA96 thi-1 hsdR17(rK-mK+) supE44 relA1 Δ(lac-proAB) [F′ traD36 proAB lacl^{q}ZΔM15]), and the cells grown on indicator LB agar, which contains isopropylthio-β-D-galactoside (IPTG) and 5-bromo-4-chloro-3-indolyl-β-D-galactoside (X-Gal). The resulting colonies were white.

As illustrated in FIG. 50, a DNA sequence was designed and synthesized as described herein, then cleaved with BamH I to provide SEQ. ID. NO.: 663, which is a 178 base sequence with 3n+1 bases flanked by BamH I sites [BamH I (3n+1) BamH I], where n=57. This DNA sequence was then inserted into the BamH I site in the MCS of the α-fragment frameshifted vector, which was transformed into E. coli JM109, and the cells grown on LB indicator agar, resulting in blue and white colonies. A blue colony was selected and the insert isolated by PCR. The sequence was confirmed by sequencing.

Those skilled in the art will understand the MCS in the α-fragment frameshifted vector described herein contains at least 13 distinct restriction sites, and consequently, in some embodiments, the DNA sequence is inserted into a different restriction site.

### EXAMPLE 7

### DNA Sequence Selection Using a Valyl-tRNA Synthesase^{ts} Frameshifted Vector

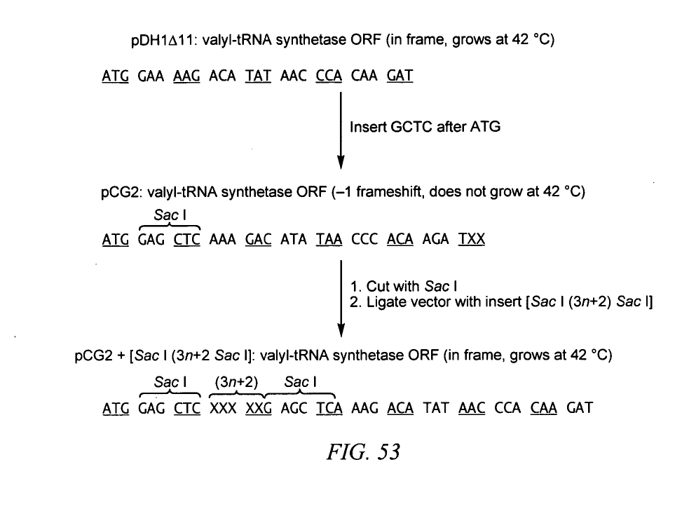

FIG. 53 illustrates the construction of a valyl-tRNA synthesase^{ts} (valS) frameshifted vector useful for identifying selecting DNA sequences by the difference in temperature at which a clone containing the correct sequence and a clone containing the incorrect sequence will grow. A frameshifted plasmid (pCG2) with an n−1 frameshift in the valS gene ORF is constructed using a pDH1Δ11 vector template (SEQ. ID. NO.: 664), which contains a wild-type valS gene. A 4 bp sequence (GCTC) is inserted immediately after the first ATG codon of the valS gene by PCR site-directed mutagenesis using the QuickChange®protocol (Strategene, La Jolla, Calif.) with PfuUltra™ DNA polymerase (Strategene, La Jolla, Calif.) and 5′-CGGCAACCTGGAAATAATGGAGCTCAAAGACATATAACCCACAAG-3′ (SEQ. ID. NO.: 665) and 5′-CTTGTGGGTTATATGTCTTTGAGCTCCATTATTTCCAGGTTGCCG-3′ (SEQ. ID. NO.: 666) as the primers. The thermocycling parameters are 1 cycle at 95° C. for 30 seconds, followed by 15 cycles at 95° C. for 30 seconds, 55° C. for 1 minute, and 68° C. for 5 minutes. The methylated pDH1Δ11 plasmid template is then removed by digestion with Dpn1 (10 U/μL) for 1 hour at 37° C. This mutagenesis protocol introduces a Sac I restriction endonuclease site as well as an n−1 frameshift in the downstream valS coding sequence of the pCG2 plasmid (SEQ. ID. NO.: 667). The frameshift prevents the expression of valS.

E. coli AB4141 contains a conditionally lethal, temperature sensitive valS gene. The strain will grow at a permissive temperature of about 37° C., but not at a restrictive temperature of about 42° C. The strain will grow at the restrictive temperature when transformed with a plasmid that expresses wild-type valS, however, for example, pDH1Δ11. Because the pCG2 plasmid does not express valS, E. coli AB4141 transformed therewith does not express wild-type valS, and consequently, does not grow at the restrictive temperature.

A method for using the pCG2 frameshifted vector to select a correct DNA sequence synthesized as described herein is illustrated in FIG. 53. The DNA sequence is designed with 3n+2 bases flanked by a pair of Sac I restriction sites [Sac I (3n+2) Sac I]. In this example, the inserted DNA sequence is CAAGAGCT, an 8 base sequence where n=0. The synthetic DNA sequence is inserted into the Sac I site of the pCG2 plasmid, the vector transformed into E. coli AB4141, and the cells cultured on LB agar at 42° C., the restrictive temperature. A correct DNA sequence inserted at the Sac I site corrects the −1 frameshift in the valS gene, thereby restoring the function of the valS gene on the plasmid. E. coli AB4141 transformed with this vector grows at the restrictive temperature. When inserted into the pCG2 plasmid, a DNA sequence with one or two (N−1) defects, however, does not correct the −1 frameshift in the valS gene. An E. coli AB4141 clone containing this plasmid does not grow at the restrictive temperature. Colonies growing at 42° C. are selected, the correct DNA sequence is isolated, and the DNA used as described herein.

The embodiments illustrated and described herein are provided as examples of certain preferred embodiments. Various changes and modifications can be made to these embodiments by those skilled in the art without departing from the teachings provided herein.

1. A method of synthesizing a DNA sequence comprising:
(i) dividing the DNA sequence recursively into small pieces of DNA, wherein adjacent pieces comprise overlapping regions;
(ii) optimizing the sequences of the pieces of DNA resulting from each recursive division to strengthen correct hybridizations and to disrupt incorrect hybridizations;
(iii) obtaining the optimized small pieces of DNA, wherein the overlapping regions of any adjacent pieces of single-stranded DNA are complementary;
(iv) combining the pieces of DNA derived from the division of the next-larger piece of DNA;
(v) allowing the pieces of DNA to self-assemble to form a DNA construct comprising single-stranded DNA segments connected by double-stranded overlap regions;
(vi) producing the next-larger piece of DNA from the DNA construct; and
(vii) repeating steps (iv), (v), and (vi) in reverse order of the recursive division in step (i) to produce the DNA sequence, wherein
at least one next-larger piece of DNA comprises a mixture of DNA molecules, the mixture comprising a correct DNA sequence and a DNA sequence comprising a point deletion, the method further comprising:
inserting the next-larger piece of DNA into a DNA insertion site in a frameshifted vector,
transforming a preselected organism with the resulting vector,
selecting an organism exhibiting a predetermined phenotype, and
isolating the next-larger piece of DNA from the selected organism, wherein
the frameshifted vector comprises an open reading frame comprising a gene and the DNA insertion site,
the gene comprises a functional portion that encodes a functional polypeptide, the expression of which changes the phenotype of the organism,
the functional portion of the gene is frameshifted such that no functional polypeptide is expressed,
the DNA insertion site is upstream of the functional portion of the gene, and
the next-larger piece of DNA, when inserted at the DNA insertion site, corrects the frameshift, such that the functional portion of the gene expresses a functional polypeptide.

2. The method of claim 1, wherein dividing the DNA sequence into small pieces of DNA is performed in a single division.

3. The method of claim 1, wherein dividing the DNA sequence into small pieces of DNA is performed in a plurality of divisions.

4. The method of claim 1, wherein the small pieces of DNA are about 60 bases long or shorter.

5. The method of claim 1, wherein the overlapping regions comprise from about 6 to about 60 base-pairs.

6. The method of claim 1, wherein optimizing comprises calculating a melting temperature for the pieces of DNA.

7. The method of claim 6, wherein the melting temperature of the lowest melting correct hybridization is at least 1° C. higher than the melting temperature of the highest melting incorrect hybridization.

8. The method of claim 1, wherein optimizing comprises adjusting boundary points between adjacent pieces of DNA.

9. The method of claim 1, wherein the next-larger piece of DNA is produced by ligating the DNA construct.

10. The method of claim 1, wherein the next-larger piece of DNA is produced by extending the DNA construct by a reaction using DNA polymerase.

11. The method of claim 1, further comprising designing a restriction site into an overlapping region.

12. The method of claim 1, wherein the change in phenotype is visually apparent.

13. The method of claim 1, wherein the frameshifted vector is a plasmid and the preselected organism is E. coli.

14. The method of claim 12, wherein the change in phenotype is color.

15. The method of claim 14, wherein the plasmid comprises a gene for the α-complementing fragment of β-galactosidase and the preselected organism is an E. coli strain with the lacZΔM15 genotype.

16. The method of claim 15, wherein the transformed E. coli is grown on indicator agar comprising isopropylthio-β-D-galactoside (IPTG) and 5-bromo-4-chloro-3-indolyl-β-D-galactoside (X-Gal), and wherein the predetermined phenotype is a blue colored colony.

17. The method of claim 15, wherein the plasmid has SEQ. ID. NO.: 661 and the preselected organism is E. coli JM109.

18. The method of claim 12, wherein the predetermined phenotype is growth at a restrictive temperature.

19. The method of claim 18, wherein the plasmid comprises a gene for valyl-tRNA synthesase^{ts} and the preselected organism is E. coli AB4141.

20. The method of claim 19, wherein the plasmid has SEQ. ID. NO.: 667.

21. The method of claim 1, wherein the frameshift is a −1 frameshift.

22. The method of claim 1, wherein the frameshift is a +1 frameshift.

23. The method of claim 1, wherein the DNA insertion site comprises a restriction site.

24. The method of claim 1, wherein the next-larger piece of DNA is an intermediate fragment.

25. The method of claim 1, wherein the next-larger piece of DNA is a full-length gene.

26. The method of claim 1, wherein the next-larger piece of DNA is isolated by polymerase chain reaction.

27. A DNA sequence synthesized according to a method comprising:
(i) dividing the DNA sequence recursively into small pieces of DNA, wherein adjacent pieces comprise overlapping regions;
(ii) optimizing the sequences of the pieces of DNA resulting from each recursive division to strengthen correct hybridizations and to disrupt incorrect hybridizations;
(iii) obtaining the optimized small pieces of DNA, wherein the overlapping regions of any adjacent pieces of single-stranded DNA are complementary;
(iv) combining the pieces of DNA derived from the division of the next larger piece of DNA;
(v) allowing the pieces of DNA to self-assemble to form a DNA construct comprising single-stranded DNA segments connected by double-stranded overlap regions;
(vi) producing the next-larger piece of DNA from the DNA construct; and
(vii) repeating steps (iv), (v), and (vi) in reverse order of the recursive division in step (i) to produce the DNA sequence, wherein
at least one next-larger piece of DNA comprises a mixture of DNA molecules, the mixture comprising a correct DNA sequence and a DNA sequence comprising a point deletion, the method further comprising:
inserting the next-larger piece of DNA into a DNA insertion site in a frameshifted vector,
transforming a preselected organism with the resulting vector,
selecting an organism exhibiting a predetermined phenotype, and
isolating the next-larger piece of DNA from the selected organism, wherein
the frameshifted vector comprises an open reading frame comprising a gene and the DNA insertion site,
the gene comprises a functional portion that encodes a functional polypeptide, the expression of which changes the phenotype of the organism,
the functional portion of the gene is frameshifted such that no functional polypeptide is expressed,
the DNA insertion site is upstream of the functional portion of the gene, and
the next-larger piece of DNA, when inserted at the DNA insertion site, corrects the frameshift, such that the functional portion of the gene expresses a functional polypeptide.

28. The DNA sequence of claim 27, wherein dividing the DNA sequence into small pieces of DNA is performed in a single division.

29. The DNA sequence of claim 27, wherein dividing the DNA sequence into small pieces of DNA is performed in a plurality of divisions.

30. The DNA sequence of claim 27, wherein the small pieces of DNA are about 60 bases long or shorter.

31. The DNA sequence of claim 27, wherein the overlapping regions comprise from about 6 to about 60 base-pairs.

32. The DNA sequence of claim 27, wherein optimizing comprises calculating a melting temperature for the pieces of DNA.

33. The DNA sequence of claim 32, wherein the melting temperature of the lowest melting correct hybridization is at least 1° C. higher than the melting temperature of the highest melting incorrect hybridization.

34. The DNA sequence of claim 27, wherein optimizing comprises adjusting boundary point between adjacent pieces of DNA.

35. The DNA sequence of claim 27, wherein the next-larger piece of DNA is produced by ligating the DNA construct.

36. The DNA sequence of claim 27, wherein the next-larger piece of DNA is produced by extending the DNA construct by a reaction using DNA polymerase.

37. The DNA sequence of claim 27, further comprising designing a restriction site into an overlapping region.

38. The DNA sequence of claim 27, wherein the change in phenotype is visually apparent

39. The DNA sequence of claim 27, wherein the frameshifted vector is a plasmid and the preselected organism is E. coli.

40. The DNA sequence of claim 38, wherein the change in phenotype is color

41. The DNA sequence of claim 40, wherein the plasmid comprises a gene for the α-complementing fragment of β-galactosidase and the preselected organism is an E. coli strain with the lacZΔM15 genotype.

42. The DNA sequence of claim 41, wherein the transformed E. coli is grown on indicator agar comprising isopropylthio-β-D-galactoside (IPTG) and 5-bromo-4-chloro-3-indolyl-β-D-galactoside (X-Gal), and wherein the predetermined phenotype is a blue colored colony.

43. The DNA sequence of claim 41, wherein the plasmid has SEQ. ID. NO.: 661 and the preselected organism is E. coli JM109.

44. The DNA sequence of claim 38, wherein the predetermined phenotype is growth at a restrictive temperature.

45. The DNA sequence of claim 44, wherein the plasmid comprises a gene for valyl-tRNA synthesase^{ts} and the preselected organism is E. coli AB4141.

46. The DNA sequence of claim 45, wherein the plasmid has SEQ. ID. NO.: 667.

47. The DNA sequence of claim 27, wherein the frameshift is a −1 frameshift.

48. The DNA sequence of claim 27, wherein the frameshift is a +1 frameshift.

49. The DNA sequence of claim 27, wherein the DNA insertion site comprises a restriction site.

50. The DNA sequence of claim 27, wherein the next-larger piece of DNA is an intermediate fragment.

51. The DNA sequence of claim 27, wherein the next-larger piece of DNA is a full-length gene.

52. The DNA sequence of claim 27, wherein the next-larger piece of DNA is isolated by polymerase chain reaction.

53. A method for isolating a piece of DNA comprising:
inserting the piece of DNA into a DNA insertion site in a frameshifted vector,
transforming a preselected organism with the resulting vector,
selecting an organism exhibiting a predetermined phenotype, and
isolating the piece of DNA from the selected organism, wherein
the frameshifted vector comprises an open reading frame comprising a gene and the DNA insertion site,
the gene comprises a functional portion that encodes a functional polypeptide, the expression of which changes the phenotype of the organism,
the functional portion of the gene is frameshifted such that no functional polypeptide is expressed,
the DNA insertion site is upstream of the functional portion of the gene, and
the piece of DNA, when inserted at the DNA insertion site, corrects the frameshift, such that the functional portion of the gene expresses a functional polypeptide.

54. The method of claim 53, wherein the change in phenotype is visually apparent.

55. The method of claim 53, wherein the frameshifted vector is a plasmid and the preselected organism is E. coli.

56. The method of claim 54, wherein the change in phenotype is color.

57. The method of claim 56, wherein the plasmid comprises a gene for the α-complementing fragment of β-galactosidase and the preselected organism is an E. coli strain with the lacZΔM15 genotype.

58. The method of claim 57, wherein the transformed E. coli is grown on indicator agar comprising isopropylthio-β-D-galactoside (IPTG) and 5-bromo-4-chloro-3-indolyl-β-D-galactoside (X-Gal), and wherein the predetermined phenotype is a blue colored colony.

59. The method of claim 57, wherein the plasmid has SEQ. ID. NO.: 661 and the preselected organism is E. coli JM109.

60. The method of claim 54, wherein the predetermined phenotype is growth at a restrictive temperature.

61. The method of claim 60, wherein the plasmid comprises a gene for valyl-tRNA synthesase^{ts} and the preselected organism is E. coli AB4141.

62. The method of claim 61, wherein the plasmid has SEQ. ID. NO.: 667.

63. The method of claim 53, wherein the frameshift is a −1 frameshift.

64. The method of claim 53, wherein the frameshift is a +1 frameshift.

65. The method of claim 53, wherein the DNA insertion site comprises a restriction site.

66. The method of claim 53, wherein the piece of DNA is an intermediate fragment.

67. The method of claim 53, wherein the piece of DNA is a full-length gene.

68. The method of claim 53, wherein piece of DNA is isolated by polymerase chain reaction.

69. A piece of DNA isolated by a method comprising:
inserting the piece of DNA into a DNA insertion site in a frameshifted vector,
transforming a preselected organism with the resulting vector,
selecting an organism exhibiting a predetermined phenotype, and
isolating the piece of DNA from the selected organism, wherein
the frameshifted vector comprises an open reading frame comprising a gene and the DNA insertion site,
the gene comprises a functional portion that encodes a functional polypeptide, the expression of which changes the phenotype of the organism,
the functional portion of the gene is frameshifted such that no functional polypeptide is expressed,
the DNA insertion site is upstream of the functional portion of the gene, and
the piece of DNA, when inserted at the DNA insertion site, corrects the frameshift, such that the functional portion of the gene expresses a functional polypeptide.

70. The piece of DNA of claim 69, wherein the change in phenotype is visually apparent.

71. The piece of DNA of claim 69, wherein the frameshifted vector is a plasmid and the preselected organism is E. coli.

72. The piece of DNA of claim 70, wherein the change in phenotype is color.

73. The piece of DNA of claim 72, wherein the plasmid comprises a gene for the α-complementing fragment of β-galactosidase and the preselected organism is an E. coli strain with the lacZΔM15 genotype.

74. The piece of DNA of claim 73, wherein the transformed E. coli is grown on indicator agar comprising isopropylthio-β-D-galactoside (IPTG) and 5-bromo-4-chloro-3-indolyl-β-D-galactoside (X-Gal), and wherein the predetermined phenotype is a blue colored colony.

75. The piece of DNA of claim 73, wherein the plasmid has SEQ. ID. NO.: 661 and the preselected organism is E. coli JM109.

76. The piece of DNA of claim 70, wherein the predetermined phenotype is growth at a restrictive temperature.

77. The piece of DNA of claim 76, wherein the plasmid comprises a gene for valyl-tRNA synthesase^{ts} and the preselected organism is E. coli AB4141.

78. The piece of DNA of claim 77, wherein the plasmid has SEQ. ID. NO.: 667.

79. The piece of DNA of claim 69, wherein the frameshift is a −1 frameshift.

80. The piece of DNA of claim 69, wherein the frameshift is a +1 frameshift.

81. The piece of DNA of claim 69, wherein the DNA insertion site comprises a restriction site.

82. The piece of DNA of claim 69, wherein the piece of DNA is an intermediate fragment.

83. The piece of DNA of claim 69, wherein the piece of DNA is a full-length gene.

84. The piece of DNA of claim 69, wherein piece of DNA is isolated by polymerase chain reaction.

85. A frameshifted vector with SEQ. ID. NO.: 661.

86. A frameshifted vector with SEQ. ID. NO.: 667.
